(19) United States
(12) Patent Application Publication
(10) Pub. No.: US 2024/0299534 A1
(43) Pub. Date: Sep. 12, 2024

(54) VACCINES FOR CORONAVIRUS PREVENTION AND TREATMENT

(71) Applicant: AUSPERBIO THERAPEUTICS INC., San Mateo, CA (US)

(72) Inventors: Guofeng CHENG, Foster City, CA (US); Cheng Yong YANG, Foster City, CA (US)

(21) Appl. No.: 18/603,039

(22) Filed: Mar. 12, 2024

Related U.S. Application Data
(63) Continuation of application No. PCT/US2022/076434, filed on Sep. 14, 2022.
(60) Provisional application No. 63/354,051, filed on Jun. 21, 2022.
(60) Provisional application No. 63/244,029, filed on Sep. 14, 2021.

(51) Int. Cl. A61K 39/215; A61K 9/127; A61K 9/51; A61K 39/00; A61P 31/14; C07K 14/005; C12N 7/00

(52) U.S. Cl. CPC A61K 39/215; A61K 9/127; A61K 9/5123; A61P 31/14; C07K 14/005; C12N 7/00; A61K 2039/53; A61K 2039/55555; A61K 2039/575; C12N 2770/20022; C12N 2770/20034

(57) ABSTRACT

The disclosure provides messenger ribonucleic acids (mRNAs) encoding an antigenic protein capable of eliciting an immune response to a coronavirus. The disclosure also provides antigen proteins encoded by the mRNAs. The disclosure further provides methods for eliciting an immune response in a subject, and methods for preventing and/or treating a coronavirus infection in a subject.

### CROSS REFERENCE TO RELATED APPLICATIONS

The present application is an international patent application which claims the benefit of and priority to the U.S. Provisional Patent Application No. 63/244,029, filed Sep. 14, 2021, and U.S. Provisional Patent Application No. 63/354,051, filed on Jun. 21, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Sep. 14, 2022, is named “127071-5001-WO_Sequence_Listing.xml” and is approximately 436 kilobytes in size.

### FIELD

The disclosure relates to messenger ribonucleic acids (mRNAs) encoding antigenic proteins capable of eliciting an immune response to a coronavirus. The disclosure also relates to the antigenic proteins encoded by mRNAs, to compositions of the mRNAs, and to vaccines comprising the mRNAs. The disclosed mRNAs and compositions thereof are suitable for preventing and/or treating coronavirus infections such as severe acute respiratory syndrome (SARS)-CoV (SARS-CoV) infections, Middle East respiratory syndrome (MERS)-CoV (MERS-CoV) infections, and/or SARS-CoV-2 infections.

Novel coronaviruses such as SARS-CoV, MERS-CoV and SARS-CoV-2 have emerged as significant threats to global public health. Unfortunately, specific medical countermeasures remain incomplete. The spi

### BACKGROUND

Novel coronaviruses such as SARS-CoV, MERS-CoV and SARS-CoV-2 have emerged as significant threats to global public health. Unfortunately, specific medical countermeasures remain incomplete. The spike (S) protein of coronaviruses such as SARS-CoV-2 has been identified as playing a key role in receptor recognition and cell membrane fusion and therefore represents a promising target for therapeutic intervention. More specifically, the SARS-CoV-2 S protein binds to a host cell by recognizing the receptor ACE2, which may promote endosome formation and triggering of viral fusion. See, e.g., Huang et al., Acta Pharmacologica Sinica, 41:1141:1149 (2020).

Ribonucleic acid (RNA) vaccines build on the knowledge that RNA (e.g., messenger RNA (Mrna)) can safely direct the body's cellular machinery to produce nearly any protein of interest. However, existing approaches may benefit from improved efficiency and specificity, and a reduction in undesirable side effects. Thus, there remains a need in the art for alternative RNA vaccines for preventing and treating coronavirus infections.

After infection by SARS-CoV-2, the immune response mounted by humans involves IgG, IgA, and IgM antibodies, among others. The IgG antibody levels generated in this immune response to wild-type SARS-CoV-2 peak on the order of 1×10¹ to 1×10² μg/Ml [FIG. 1 of Iyer et al, Sci. Immunol. 10.1126/sciimmunol.abe0367 (2020)]. Wild-type pseudovirus-based SARS-CoV-2 50% neutralization (Pvnt₅₀) titer levels generated after infection peak on the order of between about 5×10² to 2×10³ [FIG. 3 of Iyer et al, Sci. Immunol. 10.1126/sciimmunol.abe0367 (2020)].

Alternate Mrna vaccines which can generate IgG antibody levels higher than those generated by the convalescent subjects would be advantageous. Alternate Mrna vaccines which can generate pseudovirus-based SARS-CoV-2 50% neutralization (Pvnt₅₀) titer levels higher than those generated by the subjects would be advantageous. These and other advantages are provided by the current invention.

### SUMMARY

In an embodiment, the disclosure provides a messenger ribonucleic acid (Mrna) comprising: an open reading frame encoding a monomeric polypeptide chain of an antigenic protein capable of eliciting in a subject an immune response to a coronavirus, the monomeric polypeptide chain comprising a first receptor binding domain, a first linker, a hinge, and an Fc domain. In an embodiment, the disclosure provides a messenger ribonucleic acid (Mrna) comprising: an open reading frame encoding a monomeric polypeptide chain of an antigenic protein capable of eliciting in a subject an immune response to a coronavirus, the monomeric polypeptide chain comprising a first receptor binding domain, a first linker, and an Fc domain, wherein the monomeric polypeptide chain does not comprise a hinge.

In some embodiments, the antigenic protein comprises two monomeric polypeptide chains, and the two are the same. In some embodiments, the antigenic protein comprises two monomeric polypeptide chains, and the two are different.

In some embodiments, the Mrna further comprises a 5′ untranslated region (5′-UTR) and a 3′ untranslated region (3′-UTR). In some embodiments, the Mrna further comprises a poly(A) tail. In some embodiments, the Mrna further comprises a 5′ cap or 5′ cap analog. In some embodiments, the Mrna comprises a chemical modification.

In some embodiments, the Mrna comprises a ribonucleotide sequence that encodes a hinge. In some embodiments, the antigenic protein comprises two monomeric polypeptide chains, each monomeric polypeptide chain comprises a hinge, and cysteine residues in the hinge form interchain disulfide bonds between the two monomeric polypeptide chains.

In some embodiments, the Fc domain comprises a CH2 domain and a CH3 domain. In some embodiments, the Fc domain is an IgG1 or IgG4 Fc domain. In some embodiments, the IgG4 Fc domain comprises a L234A/L235A (LALA) mutation (numbering according to EU nomenclature).

In some embodiments, the first receptor binding domain is a receptor binding domain from a SARS-CoV-2 spike (S) protein. In some embodiments, the first receptor binding domain is a receptor binding domain from a SARS-CoV-2 delta variant S protein.

In some embodiments, the Mrna encodes from 5′ to 3′: the first receptor binding domain, the first linker, and the Fc domain. In some embodiments, the Mrna encodes from 5′ to 3′: the Fc domain, the first linker, and the first receptor binding domain.

In some embodiments, the Mrna further comprises a ribonucleotide sequence encoding a second linker and a second receptor binding domain. In some embodiments, the Mrna encodes from 5′ to 3′: the first receptor binding domain, the first linker, the Fc domain, the second linker, and the second receptor binding domain. In some embodiments, the first receptor binding domain and the second receptor binding domain are the same. In some embodiments, the first receptor binding domain and the second receptor binding domain are different.

In some embodiments, the Mrna further comprises a ribonucleotide sequence encoding a signal peptide. In some embodiments, the signal peptide is from a SARS-CoV-2 S protein.

In some embodiments, the first linker or the second linker is a GGGGSGGGGS (SEQ ID NO: 513), GGGGSGGGGSGGGGS (SEQ ID NO: 514), or GGGGSGGGGSGGGGSGGGGS (SEQ ID NO: 515) linker.

In some embodiments, the first receptor binding domain or the second receptor binding domain comprises amino acids 319-541 of a SARS-CoV-2 spike (S) protein. In some embodiments, the first receptor binding domain or the second receptor binding domain comprises amino acids 319-541 of a SARS-CoV-2 variant spike (S) protein. In some embodiments, the first receptor binding domain or the second receptor binding domain comprises amino acids 319-541 of a SARS-CoV-2 delta variant spike (S) protein.

In some embodiments, the mRNA sequence is as set forth in one of SEQ ID NOs: 301-319. In some embodiments, the mRNA sequence is as set forth in one of SEQ ID NOs: 321-324. In some embodiments, the mRNA sequence is as set forth in one of SEQ ID NOs: 331-372.

In an embodiment, the disclosure provides an antigenic protein encoded by the mRNA constructs as disclosed herein. In some embodiments, the disclosure provides an antigenic protein for eliciting in a subject an immune response to a coronavirus, the antigenic protein comprising two monomeric polypeptide chains, wherein each monomeric polypeptide chain comprises a first receptor binding domain, a first linker, a hinge, and an Fc domain. In some embodiments, the disclosure provides an antigenic protein for eliciting in a subject an immune response to a coronavirus, the antigenic protein comprising two monomeric polypeptide chains, wherein each monomeric polypeptide chain comprises a first receptor binding domain, a first linker, and an Fc domain, wherein the monomeric polypeptide chain does not comprise a hinge. In some embodiments, cysteine residues in the hinge form interchain disulfide bonds between the two monomeric polypeptide chains. In some embodiments, the two monomeric polypeptide chains are identical. In some embodiments, the two monomeric polypeptide chains are different.

In some embodiments, the hinge is present.

In some embodiments, the Fc domain comprises a CH2 domain and a CH3 domain. In some embodiments, the Fc domain is an IgG1 or IgG4 Fc domain. In some embodiments, the IgG4 Fc domain comprises a L234A/L235A (LALA) mutation (numbering according to Eu nomenclature).

In some embodiments, the first receptor binding domain is a receptor binding domain from a SARS-CoV-2 S protein. In some embodiments, the first receptor binding domain is receptor binding domain from a SARS-CoV-2 delta variant S protein.

In some embodiments, the antigenic protein comprises from N-terminus to C-terminus: the first receptor binding domain, the first linker, and the Fc domain. In some embodiments, the antigenic protein comprises from N-terminus to C-terminus: the Fc domain, the first linker, and the first receptor binding domain.

In some embodiments, the antigenic protein comprises a second linker and a second receptor binding domain. In some embodiments, the antigenic protein comprises from N-terminus to C-terminus: the first receptor binding domain, the first linker, the Fc domain, the second linker, and the second receptor binding domain.

In some embodiments, the antigenic protein comprises a signal peptide. In some embodiments, the signal peptide is from a SARS-CoV-2 spike protein. In some embodiments, the antigenic protein lacks a signal peptide.

In some embodiments, the first linker or the second linker is a GGGGSGGGGS (SEQ ID NO: 513), GGGGSGGGGSGGGGS (SEQ ID NO: 514), or GGGGSGGGGSGGGGSGGGGS (SEQ ID NO: 515) linker.

In some embodiments, the first receptor binding domain or the second receptor binding domain comprises amino acids 319-541 of a SARS-CoV-2 spike (S) protein. In some embodiments, the first receptor binding domain or the second receptor binding domain comprises amino acids 319-541 of a SARS-CoV-2 variant spike (S) protein. In some embodiments, the first receptor binding domain or the second receptor binding domain comprises amino acids 319-541 of a SARS-CoV-2 delta variant spike (S) protein.

In some embodiments, the monomeric polypeptide chain is encoded by one of SEQ ID NOs: 301-319. In some embodiments, the monomeric polypeptide chain is encoded by one of SEQ ID NOs: 321-324. In some embodiments, the monomeric polypeptide chain is encoded by one of SEQ ID NOs: 331-372.

In an embodiment, the disclosure provides compositions comprising the mRNA constructs as disclosed herein formulated in a lipid nanoparticle. In an embodiment, the disclosure provides vaccines comprising the mRNA constructs as disclosed herein formulated in a lipid nanoparticle. In some embodiments, the disclosure provides a vaccine comprising the compositions as disclosed herein.

In an embodiment, the disclosure provides vaccines comprising the mRNA constructs as disclosed herein. In an embodiment, the disclosure provides vaccines comprising the mRNA constructs as disclosed herein and a pharmaceutically acceptable carrier.

In an embodiment, the disclosure provides methods for eliciting an immune response in a subject in need thereof, the method comprising: providing to the subject the vaccines as disclosed herein.

In an embodiment, the disclosure provides methods of preventing and/or treating a coronavirus infection in a subject in need thereof, the method comprising administering to the subject a vaccine comprising the mRNA constructs as disclosed herein. In some embodiments, the coronavirus infection is a severe acute respiratory syndrome (SARS)-CoV (SARS-CoV) infection, a Middle East respiratory syndrome (MERS)-CoV (MERS-CoV) infection, or a SARS-CoV-2 infection.

In an embodiment, the disclosure provides methods of inducing an immune response in a subject, the methods comprising: administering to the subject a vaccine comprising the mRNA constructs as disclosed herein.

In an embodiment, the disclosure provides methods of preventing the occurrence of COVID-19 in a subject in need thereof, the method comprising administering to the subject a vaccine comprising the mRNA constructs as disclosed herein.

### BRIEF DESCRIPTION OF THE DRAWINGS

Various features of illustrative embodiments of the disclosure are described below with reference to the drawings. The illustrated embodiments are intended to illustrate, but not to limit, the disclosure.

FIG. 1 shows a schematic depicting the expression frame of a recombinant fusion protein containing a T7 transcription start site (T7 promoter), a 5′-UTR (5′UTR), a signal peptide (SP), and a 3′-UTR (3′UTR). FIG. 1 shows a “N-Fusion” configuration wherein the antigen RBD region (RBD 319-541) is fused together with the linker at the N-terminus of the fragment crystallizable region of human immunoglobulin (hIgG4 Fc).

FIG. 2 shows a schematic depicting the expression frame of a recombinant fusion protein containing a T7 transcription start site (T7 promoter), a 5′-UTR (5′UTR), a signal peptide (SP), and a 3′-UTR (3′UTR). FIG. 2 shows a “C-Fusion” configuration wherein the antigen RBD region (RBD 319-541) is fused together to the C-terminus of the fragment crystallizable region of human immunoglobulin (hIgG4 Fc) and is connected by a linker in the middle.

FIG. 3 shows a schematic depicting the expression frame of a recombinant fusion protein containing a T7 transcription start site (T7 promoter), a 5′-UTR (5′UTR), a signal peptide (SP), and a 3′-UTR (3′UTR). FIG. 3 shows a “D-Fusion” configuration containing two antigen RBD regions (RBD 319-541), wherein one antigen RBD region (RBD 319-541) is fused together with a linker at the N-terminus of the fragment crystallizable region of human immunoglobulin (hIgG4 Fc), and a second antigen RBD region (RBD 319-541) is fused together at the C-terminus of the fragment crystallizable region of human immunoglobulin (hIgG4 Fc) and is connected by a linker in the middle.

FIG. 4A and FIG. 4B show bar graphs depicting in vivo expression levels of RBD-Fc fusion proteins with different RBD lengths.

FIG. 4C and FIG. 4D depict Western blots for constructs PN19, PN20, and PN21.

FIG. 5A, FIG. 5B, and FIG. 5C show bar graphs depicting in vivo expression levels of RBD-Fc and Fc-RBD fusion proteins.

FIG. 5D depicts a Western blot for constructs PC11 and PN11 under reducing (+) and non-reducing (−) conditions.

FIG. 6A and FIG. 6B show bar graphs depicting in vivo expression levels of double RBD fusion proteins.

FIG. 6C depicts a Western blot for construct PS12 under reducing (+) and non-reducing (−) conditions.

FIG. 7A and FIG. 7B show bar graphs depicting in vitro expression levels of Fc-RBD fusion proteins with different linker lengths.

FIG. 7C and FIG. 7D depict Western blots for constructs PC8, PC9, PC10, and PC11.

FIG. 8A and FIG. 8B show bar graphs depicting in vitro expression levels of RBD-Fc fusion proteins with different linker lengths.

FIG. 8C and FIG. 8D depict Western blots for constructs PN8, PN9, PN10, PN11, PN12, PN17, and PN18.

FIG. 9A and FIG. 9B show bar graphs depicting RBD antigen levels of double RBD-Fc fusion proteins with and without a hinge.

FIG. 10A and FIG. 10B show bar graphs depicting RBD antigen levels of RBD-Fc fusion proteins with and without a hinge.

FIG. 11A shows a bar graph depicting RBD antigen expression in vitro of RBD-Fc fusion proteins with IgG1 and IgG4 Fc. FIG. 11B shows a bar graph depicting RBD antigen expression in vivo of RBD-Fc fusion proteins with IgG1 and IgG4 Fc.

FIG. 12A shows a bar graph depicting levels of RBD antibodies for the his-RBD and no tag S1 constructs. FIG. 12B shows a bar graph depicting percent inhibition of the RBD antibodies.

FIG. 13 shows a bar graph depicting RBD antigen concentration.

FIG. 14 shows a bar graph depicting RBD antigen concentration.

FIG. 15A shows serum SARS-CoV-2 anti-Delta RBD antibody levels generated in mice after administration of certain mRNA vaccine constructs (μg/mL).

FIGS. 15B and 15C show Delta and Omicron BA.1, respectively, pseudovirus-based SARS-CoV-2 50% neutralization (pVNT₅₀) titer levels generated in mice after administration of certain mRNA vaccine constructs.

FIG. 16A shows serum SARS-CoV-2 anti-Delta RBD antibody levels generated in mice after administration of certain mRNA vaccine constructs (μg/mL).

FIG. 16B shows Delta pseudovirus-based SARS-CoV-2 50% neutralization (pVNT₅₀) titer levels generated in mice after administration of certain mRNA vaccine constructs.

FIGS. 17A and 17B show serum SARS-CoV-2 anti-Delta and anti-Omicron BA.1 RBD antibody levels generated in mice after administration of certain mRNA vaccine constructs (μg/mL).

FIGS. 17C and 17D show Delta and Omicron BA.1, respectively, pseudovirus-based SARS-CoV-2 50% neutralization (pVNT₅₀) titer levels generated in mice after administration of certain mRNA vaccine constructs.

FIGS. 18A, 18B, and 18C show serum SARS-CoV-2 anti-Wild Type, anti-Delta and anti-Omicron BA.1 RBD antibody levels generated in mice after administration of certain mRNA vaccine constructs (μg/mL).

FIGS. 19A, 19B, and 19C show Delta and Omicron BA.1, respectively, pseudovirus-based SARS-CoV-2 50% neutralization (pVNT₅₀) titer levels generated in mice after administration of certain mRNA vaccine constructs.

FIGS. 20A, 20B, and 20c show the Delta, Omicron BA.1, and Omicron BA.2 pseudovirus-based SARS-CoV-2 50% neutralization (pVNT₅₀) titer levels generated in mice after administration of certain mRNA vaccine constructs.

FIGS. 21A, 21B, and 21C show serum SARS-CoV-2 anti-Delta, anti-Omicron BA.1, and anti-Omicron BA.2 RBD antibody levels generated in mice after administration of certain mRNA vaccine constructs (μg/mL).

FIGS. 21D, 21E, and 21F show the Delta, Omicron BA.1, and Omicron BA.2 pseudovirus-based SARS-CoV-2 50% neutralization (pVNT₅₀) titer levels generated in mice after administration of certain mRNA vaccine constructs.

### DETAILED DESCRIPTION

The present disclosure provides messenger ribonucleic acids (mRNAs) encoding antigenic proteins capable of eliciting in a subject an immune response to a coronavirus. Such mRNAs comprising an open reading frame encoding a monomeric polypeptide chain of the antigenic protein, wherein the monomeric polypeptide chain comprises a first receptor binding domain, a first linker, a hinge, and an Fc domain. In an exemplary embodiment, the monomeric polypeptide chain comprises a first receptor binding domain, a first linker, and an Fc domain, where the monomeric polypeptide chain does not comprise a hinge. The disclosure provides mRNAs wherein a polynucleotide sequence encoding a receptor binding domain is positioned 5′ to a polynucleotide sequence encoding an Fc domain, separated by a polynucleotide sequence encoding a linker, or wherein a polynucleotide sequence encoding a receptor binding domain is positioned 3′ to a polynucleotide sequence encoding an Fc domain, separated by a polynucleotide sequence encoding a linker. The disclosure also provides mRNAs comprising polynucleotide sequences encoding two receptor binding domains, wherein a polynucleotide sequence encoding a first receptor binding domain is positioned 5′ to a polynucleotide sequence encoding an Fc domain, separated by a polynucleotide sequence encoding a first linker, and a polynucleotide sequence encoding a second receptor binding domain is positioned 3′ to the polynucleotide sequence encoding the Fc domain, separated by a polynucleotide sequence encoding a second linker. The disclosure also provides antigenic proteins encoded by the mRNAs of the present disclosure, compositions comprising the mRNAs, vaccines comprising the mRNAs, and methods of using the mRNAs and compositions thereof in the prevention and/or treatment of coronavirus infections.

### a) Definitions

The terms “nucleic acid” and “polynucleotide” are used interchangeably herein and include any compound and/or substance that comprises a polymer of nucleotides (nucleotide monomer). Nucleic acids may be or may include, for example, ribonucleic acids (RNAs), deoxyribonucleic acids (DNAs).

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. A mRNA molecule typically includes at least one coding region. In some embodiments, the mRNAs of the disclosure include a 5′ untranslated region (UTR), a 3′ UTR, a 5′ cap and/or a poly-A tail.

The present disclosure makes use of antibody domains/regions to facilitate complex formation between two monomeric polypeptide chains. The term “antibody” or “immunoglobulin” generally refers to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized. See, e.g., Fundamental Immunology Ch. 7 (Paul, W., ed., 2nd ed. Raven Press, N.Y. (1989)). Briefly, each heavy chain typically is comprised of a heavy chain variable (VH) region and a heavy chain constant (CH) region. The CH region typically is comprised of three domains, CH1, CH2, and CH3. The heavy chains are typically inter-connected via disulfide bonds in the so-called “hinge.” Each light chain typically is comprised of a light chain variable (VL) region and a light chain constant region, the latter typically comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL region is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)).

Unless otherwise stated or contradicted by context, reference to amino acid positions in the CH or Fc region/Fc domain in the present disclosure is according to the EU-numbering (Edelman et al., Proc Natl Acad Sci USA. 1969 May; 63(1):78-85; Kabat et al., Sequences of proteins of immunological interest. 5th Edition—1991 NIH Publication No. 91-3242).

The term “hinge” as used herein refers to the hinge region of an antibody heavy chain. Thus, for example the hinge of a human IgG1 antibody corresponds to amino acids 216-230 according to the EU numbering. However, the hinge region may also be any of the other subtypes, such as IgG2, IgG3, or IgG4, as described herein.

The term “CH2 region” or “CH2 domain” as used herein refers to the CH2 region of an antibody heavy chain. Thus, for example the CH2 region of a human IgG1 antibody corresponds to amino acids 231-340 according to the EU numbering. However, the CH2 region may also be any of the other subtypes, such as IgG2, IgG3, or IgG4, as described herein.

The term “CH3 region” or “CH3 domain” as used herein refers to the CH3 region of an antibody heavy chain. Thus, for example the CH3 region of a human IgG1 antibody corresponds to amino acids 341-447 according to the EU numbering. However, the CH3 region may also be any of the other subtypes, such as IgG2, IgG3, or IgG4, as described herein. In some embodiments, amino acid 447 is present. In some embodiments, amino acid 447 is absent.

The term “Fc region” or “Fc domain” as used herein contains at least a CH2 domain and a CH3 domain of an immunoglobulin CH. The term includes native sequence Fc regions and variant Fc regions. In some embodiments, the immunoglobulin Fc fragment is selected from the immunoglobulin Fc fragment of human, mouse, rabbit, cow, goat, pig, mouse, rabbit, hamster, rat, or guinea pig. In some embodiments, the immunoglobulin Fc fragment is selected from the Fc fragment of IgG, IgA, IgD, IgE or IgM. The Fc region may be of any of the immunoglobulin subtypes, such as IgG1, IgG2, IgG3, or IgG4. In some embodiments, the immunoglobulin Fc fragment is selected from IgG1 Fc fragment, IgG2 Fc fragment, IgG3 Fc fragment, or IgG4 Fc fragment. In some embodiments, the immunoglobulin Fc fragment is a human IgG4 Fc fragment.

In an exemplary embodiment, an Fc region is typically in dimerized form via, e.g., disulfide bridges connecting the two hinges and/or non-covalent interactions between the two CH3 regions. The dimer may be a homodimer (where the two Fc region monomer amino acid sequences are identical) or a heterodimer (where the two Fc region monomer amino acid sequences differ in one or more amino acids). Preferably, the dimer is a homodimer. In some embodiments, the C-terminus lysine of the Fc domain is present. In some embodiments, the C-terminus lysine of the Fc domain is absent. In some embodiments, the Fc domain comprises one of more mutations compared to a native Fc domain. In some embodiments, the Fc domain comprises a L234A mutation and a L235A mutation (also referred to herein as “LALA mutation” or “L/A L/A”), wherein the numbering is according to EU nomenclature.

A “variant” as used herein refers to a protein or polypeptide sequence which differs in one or more amino acid residues from a parent or reference sequence. A variant may, for example, have a sequence identity of at least 80%, such as 90%, or 95%, or 97%, or 98%, or 99%, to a parent or reference sequence. Also or alternatively, a variant may differ from the parent or reference sequence by 12 or less, such as 11, 10, 9, 8, 7, 6, 5, 4, 3, 2 or 1 mutation(s) such as substitutions, insertions or deletions of amino acid residues. Accordingly, a “variant Fc region” or an “Fc region variant”, used interchangeably herein, refers to an Fc region that differs in one or more amino acid residues as compared to a parent or reference Fc region. The parent or reference Fc region is typically the Fc region of a human wild-type antibody which, depending on the context, may be a particular isotype. A variant Fc region may, in dimerized form, be a homodimer or heterodimer, e.g., where one of the amino acid sequences of the dimerized Fc region comprises a mutation while the other is identical to a parent or reference wild-type amino acid sequence. Examples of wild-type (typically a parent or reference sequence) IgG CH and variant IgG constant region amino acid sequences are disclosed, for example, in U.S. patent application Publication No. 2020/0017600.

### b) Nucleic Acids/Polynucleotides

In an exemplary embodiment, the invention provides a polynucleotide described herein. The present disclosure provides messenger ribonucleic acids (mRNAs) comprising an open reading frame encoding a monomeric polypeptide chain of an antigenic protein capable of eliciting in a subject an immune response to a coronavirus, such as an immune response to a coronavirus spike (S) protein. In an exemplary embodiment, the monomeric polypeptide chain comprises one or more receptor binding domains, one or more linker domains, a hinge, and an Fc domain. In an exemplary embodiment, the monomeric polypeptide chain comprises one or more receptor binding domains, one or more linker domains, and an Fc domain, wherein the monomeric polypeptide chain lacks a hinge. In some embodiments, the polypeptide chain comprises an antibody hinge region.

In an exemplary embodiment, the invention provides a messenger ribonucleic acid (mRNA) comprising: an open reading frame encoding a monomeric polypeptide chain comprising a first coronavirus spike protein receptor binding domain, a first linker, a Fc domain, a second linker, and a second coronavirus spike protein receptor binding domain, wherein the monomeric polypeptide chain does not comprise a hinge. In an exemplary embodiment, the invention provides a messenger ribonucleic acid (mRNA) comprising: an open reading frame encoding a monomeric polypeptide chain comprising a first coronavirus spike protein receptor binding domain, a first linker, a Fc domain, a second linker, and a second coronavirus spike protein receptor binding domain, wherein the mRNA does not comprise a ribonucleotide sequence encoding a hinge.

In an exemplary embodiment, the invention provides a messenger ribonucleic acid (mRNA) comprising: an open reading frame encoding a monomeric polypeptide chain comprising a first coronavirus spike protein receptor binding domain, a first linker, a hinge, a Fc domain, a second linker, and a second coronavirus spike protein receptor binding domain, wherein the monomeric polypeptide chain does not comprise a hinge. In an exemplary embodiment, the invention provides a messenger ribonucleic acid (mRNA) comprising a ribonucleotide sequence encoding a monomeric polypeptide chain comprising a first coronavirus spike protein receptor binding domain, a first linker, a Fc domain, a second linker, and a second coronavirus spike protein receptor binding domain. In an exemplary embodiment, the invention provides a messenger ribonucleic acid (mRNA) comprising a ribonucleotide sequence encoding a first coronavirus spike protein receptor binding domain, a first linker, a Fc domain, a second linker, and a second coronavirus spike protein receptor binding domain.

In an exemplary embodiment, the mRNA further comprises a 5′ untranslated region (5′-UTR). A “5′ untranslated region” (5′UTR) refers to a region of an mRNA that is directly upstream (i.e., 5′) from the start codon (i.e., the first codon of an mRNA transcript translated by a ribosome) that does not encode a polypeptide. In an exemplary embodiment, the 5′-UTR is described herein. In an exemplary embodiment, the 5′-UTR comprises SEQ ID NO: 24 (Z2), 25 (U1), 26 (U2), or 27 (U3). In an exemplary embodiment, the 5′-UTR is SEQ ID NO: 24 (Z2), 25 (U1), 26 (U2), or 27 (U3). In an exemplary embodiment, the 5′-UTR comprises SEQ ID NO: 25 (U1). In an exemplary embodiment, the 5′-UTR is SEQ ID NO: 25 (U1).

In an exemplary embodiment, the mRNA further comprises a 3′ untranslated region (3′-UTR). A “3′ untranslated region” (3′UTR) refers to a region of an mRNA that is directly downstream (i.e., 3′) from the stop codon (i.e., the codon of an mRNA transcript that signals a termination of translation) that does not encode a polypeptide. In an exemplary embodiment, the 3′-UTR is described herein. In an exemplary embodiment, the 3′-UTR comprises SEQ ID NO: 28 (U4), 29 (U5), or 30 (U6). In an exemplary embodiment, the 3′-UTR is SEQ ID NO: 28 (U4), 29 (U5), or 30 (U6). In an exemplary embodiment, the 3′-UTR comprises SEQ ID NO: 28 (U4). In an exemplary embodiment, the 3′-UTR is SEQ ID NO: 28 (U4).

In an exemplary embodiment, the mRNA further comprises a promoter. In an exemplary embodiment, the promoter is described herein. In an exemplary embodiment, the promoter is a T7 promoter. In an exemplary embodiment, the promoter comprises SEQ ID NO: 21 (Z1). In an exemplary embodiment, the promoter is SEQ ID NO: 21 (Z1).

In some embodiments, the mRNA further comprises a 5′ cap or 5′ cap analog. Suitable 5′ cap analogs include, but are not limited to, 3′-O-Me-m7G(5′)ppp(5′)G (ARCA); G(5′)ppp(5′)A; G(5′)ppp(5′)G; m7G(5′)ppp(5′)A; 7 mG(5′)ppp(5′)NImpNp; and m7G(5′)ppp(5′)G. In an exemplary embodiment, the 5′ cap or 5′ cap analog comprises SEQ ID NO: 22 (Z2). In an exemplary embodiment, the 5′ cap or 5′ cap analog is SEQ ID NO: 22 (Z2).

In an exemplary embodiment, the mRNA further comprises a poly(A) tail. A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation. In an exemplary embodiment, the poly(A) tail is described herein. In an exemplary embodiment, the poly(A) tail comprises SEQ ID NO: 23 (Z4). In an exemplary embodiment, the poly(A) tail is SEQ ID NO: 23 (Z4).

In some embodiments, the mRNA comprises a chemical modification. The terms “chemical modification” and “chemically modified” refer to modification with respect to adenosine (A), guanosine (G), uridine (U), thymidine (T) or cytidine (C) ribonucleosides or deoxyribonucleosides in at least one of their position, pattern, percent or population. Generally, these terms do not refer to the ribonucleotide modifications in naturally occurring 5′-terminus mRNA cap moieties. With respect to a polypeptide, the term “modification” refers to a modification relative to the canonical set 20 amino acids. Polypeptides, as provided herein, are also considered “modified” if they contain amino acid substitutions, insertions or a combination of substitutions and insertions.

Polynucleotides (e.g., RNA polynucleotides, such as mRNA polynucleotides, such as mRNA described herein), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides, such as mRNA described herein) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides, such as mRNA described herein), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminus of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides, such as mRNA described herein). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the poly-nucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

Modifications of polynucleotides (e.g., RNA polynucleotides, such as mRNA polynucleotides, such as mRNA described herein) that are useful in the vaccines of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bro-moadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP;

2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-lodo-adenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine; 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl) cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl) cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-an-hydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azido-cytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-de-oxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine e TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thio-methoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deaza-guanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueosine; Queuosine; allylamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methylpseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-di-hydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxy-acetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2 (thio)-pseudouracil; 1 (aminoalkylaminocarbonylethylenyl)-2,4-(dithio)pseudouracil; 1 (aminoalkylaminocarbonylethylenyl)-4(thio)pseudouracil; 1 (aminoalkylaminocarbonylethylenyl)-pseudouracil; 1 (aminocarbonylethylenyl)-2(thio)-pseudouracil; 1-(aminocarbonylethylenyl)-2,4-(dithio)pseudouracil; 1 (aminocarbonylethylenyl)-4 (thio)pseudouracil; 1-(aminocarbonylethylenyl)-pseudouracil; 1 substituted 2(thio)-pseudouracil; 1-substituted 2,4-(dithio)pseudouracil; 1 substituted 4 (thio) pseudouracil; 1 substituted pseudouracil; 1-(aminoalkylaminocarbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)pseudouracil; 2′ methyl, 2′ amino, 2′ azido, 2′fluoroguanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5-(2-aminopropyl)uracil; 5-(aminoalkyl)uracil; 5-(dimethylaminoalkyl)uracil; 5 (guanidiniumalkyl)uracil; 5-(methoxycarbonylmethyl)-2-(thio)uracil; 5-(methoxycarbonylmethyl)uracil; 5-(methyl) 2 (thio)uracil; 5-(methyl) 2,4 (dithio)uracil; 5 (methyl) 4 (thio)uracil; 5 (methylaminomethyl)-2(thio)uracil; 5 (methylaminom-ethyl)-2,4 (dithio)uracil; 5-(methylaminomethyl)-4 (thio)uracil; 5-(propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio) pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl) uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio) uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; ally-amino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (+) 1-(2-Hydroxypropyl)pseudouridine TP; (2R)-1-(2-Hydroxypropyl)pseudouridine TP; (2S)-1-(2-Hydroxypropyl) pseudouridine TP; (E)-5-(2-Bromo-vinyl)ara-uridine TP; (E)-5-(2-Bromo-vinyl)uridine TP; (Z)-5-(2-Bromo-vinyl) ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethylbenzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Aminopropyl)pseudo-UTP; 1-(3-Cyclopropyl-prop-2-ynyl)pseudouridine TP; 1-(4-Amino-4-carboxybutyl)pseudo-UTP; 1-(4-Aminobenzyl) pseudo-UTP; 1-(4-Aminobutyl)pseudo-UTP; 1-(4-Aminophenyl)pseudo-UTP; 1-(4-Azidobenzyl)pseudouridine TP; 1-(4-Bromobenzyl)pseudouridine TP; 1-(4-Chlorobenzyl) pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxybenzyl)pseudo-UTP; 1-(4-Methoxyphenyl)pseudo-UTP; 1-(4-Methylbenzyl)pseudouridine TP; 1-(4-Methylbenzyl)pseudo-UTP; 1-(4-Nitrobenzyl) pseudouridine TP; 1-(4-Nitrobenzyl)pseudo-UTP; 1(4-Nitrophenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Aminopentyl)pseudo-UTP; 1-(6-Aminohexyl)pseudo-UTP; 1,6-Dimethyl-pseudo-UTP; 1-[3-(2-{2-[2-(2-Aminoethoxy)-ethoxy]-ethoxy-ethoxy)-propionyllpseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoyl pseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-isopropyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2, 2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl) pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-isopropyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyluridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Tri-deuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methyl-amino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2 (2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′ amino, 2′ azido, 2′ fluoro-cytidine; 2′ methyl, 2′ amino, 2′ azido, 2′ fluoro-adenine; 2′ methyl, 2′ amino, 2′ azido, 2′ fluoro-uridine; 2′-amino-2′-deoxyribose; 2-amino-6-Chloro-purine; 2-aza-inosinyl; 2′-azido-2′-deoxyribose; 2′ fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazin-1-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanosine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA poly-nucleotides, such as mRNA polynucleotides, such as mRNA described herein) include a combination of at least two (e.g., 2, 3, 4 or more) of the aforementioned modified nucleobases.

In some embodiments, modified nucleobases in poly-nucleotides (e.g., RNA polynucleotides, such as mRNA polynucleotides, such as mRNA described herein) are selected from the group consisting of pseudouridine (ψ), N1-methylpseudouridine (m¹ψ), N1-ethylpseudouridine, 2-thiouridine, 4′-thiouridine, 5-methylcytosine, 2-thio-1-methyl-1-deaza-pseudouridine,2-thio-1-methyl-pseudouridine, 2-thio-5-aza-uridine, 2-thio-dihy-dropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides, such as mRNA described herein) are selected from the group consisting of 1-methyl-pseudouridine (m¹ψ), 5-methoxy-uridine (mo⁵U), 5-methyl-cytidine (m⁵C), pseudouridine (ψ), α-thio-guanosine and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides, such as mRNA described herein) comprise pseudouridine (ψ) and 5-methyl-cytidine (m⁵C). In some embodiments, polynucleotides (e.g., RNA polynucleotides, such as mRNA polynucleotides, such as mRNA described herein) comprise 1-methyl-pseudouridine (m¹ψ). In some embodiments, polynucleotides (e.g., RNA polynucleotides, such as mRNA polynucleotides, such as mRNA described herein) comprise 1-methyl-pseudouridine (m¹ψ) and 5-methyl-cytidine (m⁵C). In some embodiments, polynucleotides (e.g., RNA polynucleotides, such as mRNA polynucleotides, such as mRNA described herein) comprise 2-thiouridine (s²U). In some embodiments, polynucleotides (e.g., RNA polynucleotides, such as mRNA polynucleotides, such as mRNA described herein) comprise 2-thiouridine and 5-methyl-cytidine (m⁵C). In some embodiments, polynucleotides (e.g., RNA polynucleotides, such as mRNA polynucleotides, such as mRNA described herein) comprise methoxy-uridine (mo⁵U). In some embodiments, polynucleotides (e.g., RNA polynucleotides, such as mRNA polynucleotides, such as mRNA described herein) comprise 5-methoxy-uridine (mo⁵U) and 5-methyl-cytidine (m⁵C). In some embodiments, polynucleotides (e.g., RNA polynucleotides, such as mRNA polynucleotides, such as mRNA described herein) comprise 2′-O-methyl uridine. In some embodiments polynucleotides (e.g., RNA polynucleotides, such as mRNA polynucleotides, such as mRNA described herein) comprise 2′-O-methyl uridine and 5-methyl-cytidine (m⁵C). In some embodiments, polynucleotides (e.g., RNA polynucleotides, such as mRNA polynucleotides, such as mRNA described herein) comprise N6-methyl-adenosine (m⁶A). In some embodiments, polynucleotides (e.g., RNA polynucleotides, such as mRNA polynucleotides, such as mRNA described herein) comprise N6-methyl-adenosine (m⁶A) and 5-methyl-cytidine (m⁵C).

In some embodiments, polynucleotides (e.g., RNA polynucleotides, such as mRNA polynucleotides, such as mRNA described herein) are uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), and 2-thio-5-methyl-cytidine.

In some embodiments, a modified nucleobase is a modified uridine. Exemplary nucleobases and nucleosides having a modified uridine include 5-cyano uridine, and 4′-thio uridine.

In some embodiments, a modified nucleobase is a modified cytosine.

In some embodiments, a modified nucleobase is a modified adenine. Exemplary nucleobases and nucleosides having a modified adenine include 7-deaza-adenine, 1-methyl-adenosine (m¹A), 2-methyl-adenine (m²A), and N6-methyl-adenosine (m⁶A).

In some embodiments, a modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m⁷G), 1-methyl-guanosine (m¹G), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a poly-nucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the RNA (e.g., mRNA) vaccines comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxy-carbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(m⁵s²U), 1-taurinomethyl-4-thio-pseudouridine, 5-methyl-uridine (m⁵U), 1-methyl-pseudouridine 5-methyl-2-thio-uridine (m5s2U), 1-methyl-4-thio-pseudouridine (m′s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (msUm), 2′-O-methyl-pseudouridine (Wm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m3Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴2 Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2,6-di-aminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-azaadenine, 7-deaza-2-aminopurine, 7-deaza-8-aza-2-aminopurine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl) adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t6A), 2-methylthio-N6-threonylcarbamoyl-adenosine (ms²g⁶A), N6,N6-dim-ethyl-adenosine (m⁶2A), N6-hydroxynorvalylcarbamoyl-adenosine (hn⁶A), 2-methylthio-N6-hydroxynorvalylcarbamoyl-adenosine (ms2 hn6A), N6-acetyl-adenosine (ac⁶A), 7-methyl-adenine, 2-methyl-thio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶2Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (ozyW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ1), archaeosine (G*), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (mG), N2-methyl-guanosine (m²G), N2,N2-dimethyl-guanosine (m²2G), N2,7-dimethyl-guanosine (m²7G), N2,N2,7-dimethyl-guanosine (m^{2,2}7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²²Gm), 1-methyl-2′-O-methyl-guanosine (mGm), N2,7-dimethyl-2′-O-methyl-guanosine (m^{2′}7Gm), 2′-O-methyl-inosine (Im), 1,2′-O-dimethyl-inosine (m¹Im), 2′-O-ribosylguanosine (phosphate) (Gr(p)), 1-thio-guanosine, O6-methyl-guanosine, 2′-F-ara-guanosine, and 2′-F-guanosine.

In an exemplary embodiment, the chemical modifications include, but are not limited to, a 1-methylpseudouridine modification or a 1-ethylpseudouridine modification.

In some embodiments, the polynucleotides of the present disclosure are codon optimized. Codon optimization methods are known in the art and, in some embodiments, may be used to match codon frequencies in target and host organisms to: ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art, non-limiting examples of which include services from GeneArt (Life Technologies), DNA2.0 (Menlo Park Calif.) and/or proprietary methods. In some embodiments, the open reading frame (ORF) sequence is optimized using optimization algorithms.

### b1) Encoding Signal Peptide

In some embodiments, the mRNA comprises a ribonucleotide sequence encoding a signal peptide. In some embodiments, antigenic polypeptides encoded by RNA (e.g., mRNA described herein) polynucleotides comprise a signal peptide. Signal peptides are typically needed for the translocation across the membrane on the secretory pathway and, thus, universally control the entry of most proteins both in eukaryotes and prokaryotes to the secretory pathway. Signal peptides generally include three regions: an N-terminus region of differing length, which usually comprises positively charged amino acids; a hydrophobic region; and a short carboxy-terminus peptide region. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane. The signal peptide, however, is not responsible for the final destination of the mature protein. Secretory proteins devoid of additional address tags in their sequence are by default secreted to the external environment. During recent years, a more advanced view of signal peptides has evolved, showing that the functions and immunodominance of certain signal peptides are much more versatile than previously anticipated.

Vaccines of the present disclosure may comprise, for example, RNA (e.g., mRNA described herein) polynucleotides encoding an artificial signal peptide, wherein the signal peptide coding sequence is operably linked to and is in frame with the coding sequence of the antigenic polypeptide. Thus, vaccines of the present disclosure, in some embodiments, produce an antigenic polypeptide comprising an antigenic polypeptide partly derived from, for example, SARS-CoV, MERS-CoV, and/or SARS-CoV-2, fused to a signal peptide. In some embodiments, a signal peptide is fused to the N-terminus of the antigenic polypeptide. In some embodiments, a signal peptide is fused to the C-terminus of the antigenic polypeptide.

The ribonucleotide sequence encoding a signal peptide may have a length of 30-60 ribonucleotides. For example, the ribonucleotide sequence encoding a signal peptide may have a length of 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ribonucleotides. In some embodiments, the ribonucleotide sequence encoding a signal peptide may have a length of 30-35, 35-40, 40-45, 45-50, 50-55, 55-60, 30-40, 40-50, 50-60, 35-45, 45-55, 30-45, 35-50, 40-55, 45-60, 30-50, 30-55, 35-55, 35-60, 40-60, 30-33, 33-36, 36-39, 39-42, 42-45, 45-48, 48-51, 51-54, 54-57, or 57-60 ribonucleotides. In some embodiments, the ribonucleotide sequence encoding a signal peptide may have a length of 35-55 ribonucleotides. In an exemplary embodiment, the ribonucleotide sequence encoding the signal peptide comprises a chemical modification.

A signal peptide may have a length of 5-25 amino acids. For example, a signal peptide may have a length of 5, 6, 7, 8, 9, 10, 11, 12, 13, 14, 15, 16, 17, 18, 19, 20, 21, 22, 23, 24, or 25 amino acids. In some embodiments, a signal peptide has a length of 5-7, 7-12, 12-17, 17-22, 22-25, 10-18, 18-25, 5-9, 9-16, 16-23, 23-25, 5-10, 10-14, 14-18, 18-22, 10-15, 15-20, 20-25, 5-8, 8-12, 12-16, 16-20, 20-24, 10-20, 5-15, 15-25, or 10-25 amino acids. In some embodiments, a signal peptide has a length of 12-18 amino acids. In some embodiments, a signal peptide is cleaved from the nascent polypeptide at the cleavage junction during ER processing. In some embodiments, a signal peptide is present on the polypeptide after the polypeptide has left the cell. The mature antigenic polypeptide produced by a RNA (e.g., mRNA) vaccine of the present disclosure typically does not comprise a signal peptide. The examples disclosed herein are not meant to be limiting and any signal peptide that is known in the art to facilitate targeting of a protein to ER for processing and/or targeting of a protein to the cell membrane may be used in accordance with the present disclosure.

In some embodiments, the signal peptide fused to the antigenic polypeptide is an artificial signal peptide. In some embodiments, the signal peptide is a signal peptide from a SARS-CoV-2 S protein, a signal peptide from an influenza hemagglutinin (HA) protein, a signal peptide from a vesicular stomatitis virus G (VSV-G) protein, a signal peptide from an albumin (e.g., a human serum albumin (HSA)) protein, or a signal peptide from a human IgG2 heavy chain. In some embodiments, the signal peptide is a signal peptide from a SARS-CoV-2 S protein. In some embodiments, the signal peptide is a signal peptide from a SARS-CoV-2 S protein alpha variant, beta variant, delta variant, or gamma variant. In some embodiments, the signal peptide is a signal peptide from a SARS-CoV-2 S protein delta variant.

In an exemplary embodiment, the mRNA further comprises a ribonucleotide sequence encoding a signal peptide. In an exemplary embodiment, the signal peptide is encoded by a ribonucleotide sequence comprising SEQ ID NO: 1, 2, 3, 4, 5, 6, 7, 8, 9, 10, 11, 12, 13, 14, or 15 (A1-A15). In an exemplary embodiment, the signal peptide is a coronavirus spike protein signal peptide. In an exemplary embodiment, the coronavirus spike protein signal peptide is encoded by a ribonucleotide sequence comprising SEQ ID NO: 1, 4, 5, 6, 7, 8, 9, 10, 11, 12, 13, 14, or 15 (A1, A4-A15). In an exemplary embodiment, the signal peptide is encoded by a ribonucleotide sequence comprising SEQ ID NO: 1, 3, or 11 (A1, A3, and A11). In an exemplary embodiment, the signal peptide is encoded by a ribonucleotide sequence comprising SEQ ID NO: 1 (A1). In an exemplary embodiment, the coronavirus spike protein signal peptide is a SARS-CoV-2 wild-type signal peptide, SARS-CoV-2 delta signal peptide, SARS-CoV-2 omicron BA.1 signal peptide, SARS-CoV-2 omicron BA.1.1 signal peptide, or a SARS-CoV-2 omicron BA.2 signal peptide. In an exemplary embodiment, the coronavirus spike protein signal peptide is a SARS-CoV-2 delta signal peptide. In an exemplary embodiment, the coronavirus spike protein signal peptide comprises SEQ ID NO: 501 (S1). In an exemplary embodiment, the coronavirus spike protein signal peptide comprises SEQ ID NO: 504 (S4).

### b2) Encoding Linkers

In an exemplary embodiment, the mRNA comprises a ribonucleotide sequence encoding a linker. The mRNAs described herein can encode antigenic proteins which contain parts from two or more genes (RBD, Fc domain, hinge) synthesized as a single multifunctional construct. The separation distance between functional units can impact their access to their targets or partners. In an exemplary embodiment, the linkers described herein contain small, non-polar or polar residues such as Gly, Ser and Thr. The most common is the (Gly4Ser)n linker (Gly-Gly-Gly-Gly-Ser)n, where n indicates the number of repeats of the motif. In an exemplary embodiment, the mRNA comprises a ribonucleotide sequence encoding a first linker. In an exemplary embodiment, the mRNA comprises a ribonucleotide sequence encoding a first linker and a second linker.

The ribonucleotide sequence encoding a linker may have a length of 30-50 ribonucleotides. For example, the ribonucleotide sequence encoding a linker may have a length of 30, 31, 32, 33, 34, 35, 36, 37, 38, 39, 40, 41, 42, 43, 44, 45, 46, 47, 48, 49, or 50 ribonucleotides. In some embodiments, the ribonucleotide sequence encoding a linker may have a length of 30-35, 35-40, 40-45, 45-50, 30-40, 40-50, 35-45, 30-45, 35-50, 30-33, 33-36, 36-39, 39-42, 42-45, 45-48, or 48-50 ribonucleotides. In some embodiments, the ribonucleotide sequence encoding a linker may have a length of 35-40 ribonucleotides. In an exemplary embodiment, the ribonucleotide sequence encoding the linker comprises a chemical modification.

A linker may have a length of 2-25 amino acids. For example, a linker may have a length of 2, 3, 4, 5, 6, 7, 8, 9, 10, 11, 12, 13, 14, 15, 16, 17, 18, 19, 20, 21, 22, 23, 24, or 25 amino acids. In some embodiments, a linker has a length of 2-7, 7-12, 12-17, 17-22, 22-25, 2-10, 10-18, 18-25, 2-9, 9-16, 16-23, 23-25, 2-5, 5-10, 10-15, 15-20, 20-25, 8-12, 12-16, 16-20, 20-24, 10-20, 5-15, or 15-25 amino acids. The linker may have a length of 5-20 amino acids.

In an exemplary embodiment, the first linker and the second linker each comprise GGGGS. In an exemplary embodiment, the first linker or the second linker comprise (GGGGS)n, wherein n is 1 or 2 or 3 or 4 or 5. In an exemplary embodiment, the first linker or the second linker comprise (GGGS(GGGGS))n, wherein n is 1 or 2 or 3 or 4 or 5. In an exemplary embodiment, the first linker and the second linker are each independently encoded by a ribonucleotide sequence comprising SEQ ID NO: 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or 68 (D1-D38). In an exemplary embodiment, the first linker is encoded by a ribonucleotide sequence comprising SEQ ID NO: 33, 34, 35, 36, 37, 38, 39, 40, 41, 42, 43, 44, 45, 46, or 47 (D3-D17). In an exemplary embodiment, the first linker is encoded by a ribonucleotide sequence comprising SEQ ID NO: 31, 33, 35, 45, 48, 50, or 52 (D1, D3, D5, D15, D18, D20, D22). In an exemplary embodiment, the first linker is encoded by a ribonucleotide sequence comprising SEQ ID NO: 33 or 45 (D3, D15). In an exemplary embodiment, the second linker is encoded by a ribonucleotide sequence comprising SEQ ID NO: 57, 58, 59, 60, 61, 62, 63, 64, 65, or 67 (D27-D37). In an exemplary embodiment, the second linker is encoded by a ribonucleotide sequence comprising SEQ ID NO: 33, 36, 57, or 66 (D3, D6, D27, D36). In an exemplary embodiment, the second linker is encoded by a ribonucleotide sequence comprising SEQ ID NO: 57 or 66 (D27, D36). In an exemplary embodiment, the first linker and the second linker each independently comprise SEQ ID NO: 511, 512, 513, 514, 515, or 516 (K1-K6). In an exemplary embodiment, the first linker comprises SEQ ID NO: 513 (K3). In an exemplary embodiment, the second linker comprises SEQ ID NO: 515 (K5).

### b3) Encoding Hinge

In an exemplary embodiment, the mRNA comprises a ribonucleotide sequence encoding a hinge. In an exemplary embodiment, the hinge is a region derived from an analogous portion of an IgG that links the Fab (antigen binding fragment) and the Fc (crystallizable fragment) of the immunoglobulin.

The ribonucleotide sequence encoding a hinge may have a length of 95-130 ribonucleotides. For example, the ribonucleotide sequence encoding a hinge may have a length of 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 ribonucleotides. In some embodiments, the ribonucleotide sequence encoding a hinge may have a length of 95-130, 100-130, 105-130, 110-130, 115-130, 120-130, 125-130, 95-100, 95-105, 95-110, 95-115, 95-120, 95-125, 100-105, 100-110, 100-115, 100-120, 100-125, 110-115, 110-120, 110-125, 115-120, 115-125, or 120-125 ribonucleotides. In some embodiments, the ribonucleotide sequence encoding a hinge may have a length of 105-120 ribonucleotides. In an exemplary embodiment, the ribonucleotide sequence encoding the hinge comprises a chemical modification.

A hinge may have a length of 35-45 amino acids. For example, a hinge may have a length of 35, 36, 37, 38, 39, 40, 41, 42, 43, 44, or 45 amino acids. In some embodiments, a hinge has a length of 35-40, 40-45, 35-39, 39-45, 41-45, 35-38, 38-41, 41-44, 42-45, 35-37, 36-38, 37-39, 38-40, 39-41, 40-42, 41-43, 42-44, or 43-45 amino acids. The hinge may have a length of 35-45 amino acids.

In an exemplary embodiment, the mRNA comprises a ribonucleotide sequence encoding a hinge derived from IgG1. In an exemplary embodiment, the mRNA comprises a ribonucleotide sequence encoding a hinge derived from IgG4. In an exemplary embodiment, the hinge is encoded by a ribonucleotide sequence comprising SEQ ID NO: 81, 82, 83, 84, 85, 86, 87, 88, 89, 90, 91, 92, 93, 94, 95, 96, 97, or 98. (H1-H17) In an exemplary embodiment, the hinge is encoded by a ribonucleotide sequence comprising SEQ ID NO: 81, 91, or 96. (H1, H11, H16) In an exemplary embodiment, the hinge is encoded by a ribonucleotide sequence comprising SEQ ID NO: 81 or 91. (H1, H11) In an exemplary embodiment, the hinge comprises SEQ ID NO: 521, 522, 523, 524, 525, 526, or 527. (I1-I7) In an exemplary embodiment, the hinge comprises SEQ ID NO: 521. (I1)

### b4) Encoding Fc Domain

In an exemplary embodiment, the mRNA comprises a ribonucleotide sequence encoding a Fc domain. In an exemplary embodiment, the mRNA comprises a ribonucleotide sequence encoding an IgG Fc domain.

The ribonucleotide sequence encoding a Fc domain may have a length of 550-600 ribonucleotides. For example, the ribonucleotide sequence encoding a Fc domain may have a length of 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 ribonucleotides. In some embodiments, the ribonucleotide sequence encoding a Fc domain may have a length of 550-555, 555-560, 560-565, 565-570, 570-575, 575-580, 580-585, 585-590, 590-595, 595-600, 550-560, 555-565, 560-570, 565-575, 570-580, 575-585, 590-600, 550-565, 555-570, 560-575, 565-580, 570-585, 575-590, 580-595, 585-600, 550-570, 555-575, 560-580, 565-585, 570-590, 575-595, or 580-600 ribonucleotides. In some embodiments, the ribonucleotide sequence encoding a Fc domain may have a length of 570-580 ribonucleotides. In an exemplary embodiment, the ribonucleotide sequence encoding the Fc domain comprises a chemical modification.

A Fc domain may have a length of 180-200 amino acids. For example, a Fc domain may have a length of 180, 181, 182, 183, 184, 185, 186, 187, 188, 189, 190, 191, 192, 193, 194, 195, 196, 197, 198, 199, or 200 amino acids. In some embodiments, a Fc domain has a length of 180-182, 182-184, 184-186, 186-188, 188-190, 190-192, 192-194, 194-196, 196-198, 198-200, 180-185, 185-190, 190-195, or 195-200 amino acids. The Fc domain may have a length of 190-195 amino acids.

In an exemplary embodiment, the Fc domain comprises a CH2 domain and a CH3 domain. In an exemplary embodiment, the Fc domain is an IgG1 or IgG4. In an exemplary embodiment, the Fc domain is IgG1 comprising a S to P mutation. In an exemplary embodiment, the Fc domain is IgG4 comprising a S to P mutation. In an exemplary embodiment, the Fc domain is IgG1 comprising a S228P mutation. In an exemplary embodiment, the Fc domain is IgG4 comprising a S228P mutation. In an exemplary embodiment, the Fc domain is IgG4 comprising a F234A mutation. In an exemplary embodiment, the Fc domain is IgG4 comprising a L235A mutation. In an exemplary embodiment, the Fc domain is IgG4 comprising a L234A/L235A (LALA) mutation. In an exemplary embodiment, the Fc domain is encoded by a ribonucleotide sequence comprising SEQ ID NO: 111, 112, 113, 114, 115, 116, 117, 118, 119, 120, 121, 122, 123, 124, 125, 126, 127, 128, 129, 130, 131, or 132. (F1-F22). In an exemplary embodiment, the Fc domain is encoded by a ribonucleotide sequence comprising SEQ ID NO: 111, 115, 117, 122, or 129. (F1, F5, F7, F12, F19). In an exemplary embodiment, the Fc domain is encoded by a ribonucleotide sequence comprising SEQ ID NO: 115 or 129. (F5, F19). In an exemplary embodiment, the Fc domain comprises SEQ ID NO: 541, 542, 543, 544, 545, 546, 547, or 548. (G1-G8). In an exemplary embodiment, the Fc domain comprises SEQ ID NO: 541, 545, or 547. (G1, G5, G7). In an exemplary embodiment, the Fc domain comprises SEQ ID NO: 545. (G5)

### b5) Encoding Coronavirus Spike Protein RBD Area

In an exemplary embodiment, the mRNA comprises a ribonucleotide sequence encoding a coronavirus spike protein receptor binding domain (RBD). In an exemplary embodiment, the mRNA comprises a ribonucleotide sequence encoding a SARS-CoV-2 spike protein receptor binding domain. SARS-CoV-2 contains four structural proteins: spike (S), nucleocapsid (N), envelope (E) and membrane (M). The S protein can be responsible for viral attachment, fusion and entry. The S protein has two domains, S1 and S2, which are responsible for the attachment step. The S1 domain is involved in host cell receptor recognition and binding whereas S1 domain contain the putative fusion peptide as well as heptad repeat HR1 and HR2. In most cases, the S1 contains the RBD.

In an exemplary embodiment, the mRNA comprises a ribonucleotide sequence encoding a coronavirus spike protein receptor binding domain (RBD) area. In an exemplary embodiment, the mRNA comprises a ribonucleotide sequence encoding a SARS-CoV-2 spike protein receptor binding domain (RBD) area. As used herein, the term “receptor binding domain (RBD) area” refers to a coronavirus spike protein receptor binding domain (RBD), such as a SARS-CoV-2 RBD, as well as a portion of the S1 domain on one side, or both sides, of the RBD. In an exemplary embodiment, the RBD area comprises, from 5′ to 3′: 1-5, 5-10, 10-15, 15-20, 20-25, 25-30, 30-35, 35-40, 40-45, 45-50, 50-55, 55-60, 60-65, 65-70, 70-75, 75-80, 80-85, or 85-90 nucleotides of the S1 domain adjacent to the RBD, the RBD, and 1-5, 5-10, 10-15, 15-20, 20-25, 25-30, 30-35, 35-40, 40-45, 45-50, 50-55, 55-60, 60-65, 65-70, 70-75, 75-80, 80-85, or 85-90 nucleotides of the S1 domain adjacent to the RBD. In an exemplary embodiment, the RBD area comprises, from 5′ to 3′: 1-5, 5-10, 10-15, 15-20, 20-25, or 25-30 nucleotides of the S1 domain adjacent to the RBD, the RBD, and 1-5, 5-10, 10-15, 15-20, 20-25, or 25-30 nucleotides of the S1 domain adjacent to the RBD. In an exemplary embodiment, the RBD area comprises, from 5′ to 3′: 1-5, 5-10, 10-15, 15-20, 20-25, 25-30, 30-35, 35-40, 40-45, 45-50, 50-55, 55-60, 60-65, 65-70, 70-75, 75-80, 80-85, or 85-90 nucleotides of the S1 domain adjacent to the RBD, and the RBD. In an exemplary embodiment, the RBD area comprises, from 5′ to 3′: 1-5, 5-10, 10-15, 15-20, 20-25, or 25-30 nucleotides of the S1 domain adjacent to the RBD, and the RBD. In an exemplary embodiment, the RBD area comprises, from 5′ to 3′: the RBD, and 1-5, 5-10, 10-15, 15-20, 20-25, 25-30, 30-35, 35-40, 40-45, 45-50, 50-55, 55-60, 60-65, 65-70, 70-75, 75-80, 80-85, or 85-90 nucleotides of the S1 domain adjacent to the RBD. In an exemplary embodiment, the RBD area comprises, from 5′ to 3′: the RBD, and 1-5, 5-10, 10-15, 15-20, 20-25, or 25-30 nucleotides of the S1 domain adjacent to the RBD. In an exemplary embodiment, the RBD area comprises, from N-terminus to C-terminus: 1-5, 5-10, 10-15, 15-20, 20-25, 25-30, 30-35, or 35-40 amino acids of the S1 domain adjacent to the RBD, the RBD, and 1-5, 5-10, 10-15, 15-20, 20-25, 25-30, 30-35, or 35-40 amino acids of the S1 domain adjacent to the RBD. In an exemplary embodiment, the RBD area comprises, from N-terminus to C-terminus: 1-5 or 5-10 amino acids of the S1 domain adjacent to the RBD, the RBD, and 1-5 or 5-10 amino acids of the S1 domain adjacent to the RBD. In an exemplary embodiment, the RBD area comprises, from N-terminus to C-terminus: 1-5, 5-10, 10-15, 15-20, 20-25, 25-30, 30-35, or 35-40 amino acids of the S1 domain adjacent to the RBD, and the RBD. In an exemplary embodiment, the RBD area comprises, from N-terminus to C-terminus: 1-5 or 5-10 amino acids of the S1 domain adjacent to the RBD, and the RBD. In an exemplary embodiment, the RBD area comprises, from N-terminus to C-terminus: the RBD, and 1-5, 5-10, 10-15, 15-20, 20-25, 25-30, 30-35, or 35-40 amino acids of the S1 domain adjacent to the RBD. In an exemplary embodiment, the RBD area comprises, from N-terminus to C-terminus: the RBD, and 1-5 or 5-10 amino acids of the S1 domain adjacent to the RBD.

In an exemplary embodiment, the mRNA comprises a ribonucleotide sequence encoding a coronavirus spike protein RBD. In an exemplary embodiment, the mRNA comprises a ribonucleotide sequence encoding a first coronavirus spike protein RBD. In an exemplary embodiment, the mRNA comprises a ribonucleotide sequence encoding a first coronavirus spike protein RBD and a second coronavirus spike protein RBD. In an exemplary embodiment, the first coronavirus spike protein RBD is a SARS-CoV-2 spike protein RBD. In an exemplary embodiment, the first coronavirus spike protein RBD is a SARS-CoV-2 wild-type spike protein RBD, a SARS-CoV-2 beta spike protein RBD, a SARS-CoV-2 lambda spike protein RBD, a SARS-CoV-2 delta spike protein RBD, a SARS-CoV-2 mu spike protein RBD, a SARS-CoV-2 omicron BA. 1 spike protein RBD, a SARS-CoV-2 omicron BA.1.1 spike protein RBD, or a SARS-CoV-2 omicron BA.2 spike protein RBD. In an exemplary embodiment, the first coronavirus spike protein RBD is a SARS-CoV-2 delta spike protein RBD or a SARS-CoV-2 omicron BA.2 spike protein RBD. In an exemplary embodiment, the second coronavirus spike protein RBD is a SARS-CoV-2 spike protein RBD. In an exemplary embodiment, the second coronavirus spike protein RBD is a SARS-CoV-2 wild-type spike protein RBD, a SARS-CoV-2 beta spike protein RBD, a SARS-CoV-2 lambda spike protein RBD, a SARS-CoV-2 delta spike protein RBD, a SARS-CoV-2 mu spike protein RBD, a SARS-CoV-2 omicron BA. 1 spike protein RBD, a SARS-CoV-2 omicron BA. 1.1 spike protein RBD, or a SARS-CoV-2 omicron BA.2 spike protein RBD. In an exemplary embodiment, the second coronavirus spike protein RBD is a SARS-CoV-2 delta spike protein RBD or a SARS-CoV-2 omicron BA.2 spike protein RBD.

In an exemplary embodiment, the mRNA comprises a ribonucleotide sequence encoding a coronavirus spike protein receptor binding domain (RBD) area. In an exemplary embodiment, the mRNA comprises a ribonucleotide sequence encoding a first coronavirus spike protein receptor binding domain (RBD) area. In an exemplary embodiment, the mRNA comprises a ribonucleotide sequence encoding a first coronavirus spike protein receptor binding domain (RBD) area and a second coronavirus spike protein receptor binding domain (RBD) area. In an exemplary embodiment, the first coronavirus spike protein RBD area is a SARS-CoV-2 spike protein RBD area. In an exemplary embodiment, the first coronavirus spike protein RBD area is a SARS-CoV-2 wild-type spike protein RBD area, a SARS-CoV-2 beta spike protein RBD area, a SARS-CoV-2 lambda spike protein RBD area, a SARS-CoV-2 delta spike protein RBD area, a SARS-CoV-2 mu spike protein RBD area, a SARS-CoV-2 omicron BA. 1 spike protein RBD area, a SARS-CoV-2 omicron BA.1.1 spike protein RBD area, or a SARS-CoV-2 omicron BA.2 spike protein RBD area. In an exemplary embodiment, the first coronavirus spike protein RBD area is a SARS-CoV-2 delta spike protein RBD area or a SARS-CoV-2 omicron BA.2 spike protein RBD area. In an exemplary embodiment, the second coronavirus spike protein RBD area is a SARS-CoV-2 spike protein RBD area. In an exemplary embodiment, the second coronavirus spike protein RBD area is a SARS-CoV-2 wild-type spike protein RBD area, a SARS-CoV-2 beta spike protein RBD area, a SARS-CoV-2 lambda spike protein RBD area, a SARS-CoV-2 delta spike protein RBD area, a SARS-CoV-2 mu spike protein RBD area, a SARS-CoV-2 omicron BA.1 spike protein RBD area, a SARS-CoV-2 omicron BA.1.1 spike protein RBD area, or a SARS-CoV-2 omicron BA.2 spike protein RBD area. In an exemplary embodiment, the second coronavirus spike protein RBD area is a SARS-CoV-2 delta spike protein RBD area or a SARS-CoV-2 omicron BA.2 spike protein RBD area.

In an exemplary embodiment, the mRNA of section b5) has a length of 570-730 ribonucleotides. For example, the mRNA of section b5) may have a length of 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, or 730 ribonucleotides. In some embodiments, the mRNA of section b5) may have a length of 570-590, 575-595, 580-600, 585-605, 590-610, 595-615, 600-620, 605-625, 610-630, 615-635, 620-640, 625-645, 630-650, 635-655, 640-660, 645-665, 650-670, 655-675, 660-680, 665-685, 670-690, 675-695, 680-700, 685-705, 690-710, 695-715, 700-720, 705-725, or 710-730 ribonucleotides. In an exemplary embodiment, the mRNA of section b5) comprises a chemical modification.

The sequences of section b5) have a length of 185-250 amino acids. For example, the sequences of section b5) have a length of 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, or 240 amino acids. In some embodiments, the sequences of section b5) have a length of 185-190, 190-195, 195-200, 200-205, 205-210, 210-215, 215-220, 220-225, 225-230, 230-235, 235-240, 240-245, 245-250, 185-195, 190-200, 195-205, 200-210, 205-215, 210-220, 215-225, 220-230, 225-235, 230-240, 235-245, 240-250, 185-200, 190-205, 195-210, 200-215, 205-220, 210-225, 215-230, 220-235, 225-240, 230-245, 235-250, 185-205, 190-210, 195-215, 200-220, 205-225, 210-230, 215-235, 220-240, 225-245, or 230-250 amino acids. In some embodiments, the sequences of section b5) have a length of 200-225 amino acids.

In an exemplary embodiment, the first coronavirus spike protein receptor binding domain and the second coronavirus spike protein receptor binding domain are each independently encoded by a ribonucleotide sequence comprising SEQ ID NO: 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or 201. (B1-B51). In an exemplary embodiment, the first coronavirus spike protein receptor binding domain and the second coronavirus spike protein receptor binding domain are each independently encoded by a ribonucleotide sequence comprising SEQ ID NO: 151, 154, 159, 162, 164, 165, 166, 167, 168, 169, 170, 171, 172, 173, 175, 180, 181, 192, or 193. (B1, B4, B9, B12, B14, B15, B16, B17, B18, B19, B20, B21, B22, B23, B25, B30, B31, B42, or B43). In an exemplary embodiment, the first coronavirus spike protein receptor binding domain is encoded by a ribonucleotide sequence comprising SEQ ID NO: 154, 159, 162, 164, 165, 180, or 192. (B4, B9, B12, B14, B15, B30, or B42). In an exemplary embodiment, the first coronavirus spike protein receptor binding domain is encoded by a ribonucleotide sequence comprising SEQ ID NO: 159 or 192. (B9 or B42). In an exemplary embodiment, the second coronavirus spike protein receptor binding domain is encoded by a ribonucleotide sequence comprising SEQ ID NO: 151, 154, 166, 167, 168, 169, 170, 171, 172, 173, 175, 181, or 193. (B1, B4, B16, B17, B18, B19, B20, B21, B22, B23, B25, B31, or B43). In an exemplary embodiment, the second coronavirus spike protein receptor binding domain is encoded by a ribonucleotide sequence comprising SEQ ID NO: 151, 166, 167, 169, 170, 172, 173, or 193. (B1, B16, B17, B19, B20, B22, B23, or B43). In an exemplary embodiment, the first coronavirus spike protein receptor binding domain and the second coronavirus spike protein receptor binding domain each independently comprise SEQ ID NO: 561, 562, 563, 564, 565, 566, 567, 568, 569, 570, 571, 572, 573, 574, 575, 576, 577, 578, 579, 580, or 581. (R1-R21). In an exemplary embodiment, the first coronavirus spike protein receptor binding domain comprises SEQ ID NO: 564, 569, 571, or 572. (R4, R9, R11, or R12). In an exemplary embodiment, the first coronavirus spike protein receptor binding domain comprises SEQ ID NO: 564 or 572. (R4 or R12). In an exemplary embodiment, the first coronavirus spike protein receptor binding domain comprises SEQ ID NO: 564. (R4). In an exemplary embodiment, the second coronavirus spike protein receptor binding domain comprises SEQ ID NO: 561, 564, 572, 573, 574, 575, 576, 577, 578, or 579. (R1, R4, R12, R13, R14, R15, R16, R17, R18, or R19). In an exemplary embodiment, the second coronavirus spike protein receptor binding domain comprises SEQ ID NO: 561, 564, 572, 573, 575, 576, 578, or 579. (R1, R4, R12, R13, R15, R16, R18, or R19). In an exemplary embodiment, the second coronavirus spike protein receptor binding domain comprises SEQ ID NO: 579. (R19).

### b6) Combinations/Embodiments

In an exemplary embodiment, the mRNA encodes from 5′ to 3′: a receptor binding domain, a linker, a hinge, and an Fc domain. In an exemplary embodiment, the mRNA encodes from 5′ to 3′: a receptor binding domain, a linker, and an Fc domain, wherein the mRNA does not encode for a hinge. In an exemplary embodiment, the mRNA encodes from 5′ to 3′: a hinge, an Fc domain, a linker, and a receptor binding domain. In an exemplary embodiment, the mRNA encodes from 5′ to 3′: an Fc domain, a linker, and a receptor binding domain, wherein the mRNA does not encode for a hinge.

In an exemplary embodiment, the mRNA encodes from 5′ to 3′: a signal peptide, a receptor binding domain, a linker, a hinge, and an Fc domain. In an exemplary embodiment, the mRNA encodes from 5′ to 3′: a signal peptide, a receptor binding domain, a linker, and an Fc domain, wherein the mRNA does not encode for a hinge. In an exemplary embodiment, the mRNA encodes from 5′ to 3′: a signal peptide, a hinge, an Fc domain, a linker, and a receptor binding domain. In an exemplary embodiment, the mRNA encodes from 5′ to 3′: a signal peptide, an Fc domain, a linker, and a receptor binding domain, wherein the mRNA does not encode for a hinge.

In an exemplary embodiment, the mRNA encodes from 5′ to 3′: a signal peptide, a RBD area, a linker, a hinge, and an Fc domain. In an exemplary embodiment, the mRNA encodes from 5′ to 3′: a signal peptide, a RBD area, a linker, and an Fc domain, wherein the mRNA does not encode for a hinge. In an exemplary embodiment, the mRNA encodes from 5′ to 3′: a signal peptide, a hinge, an Fc domain, a linker, and a RBD area. In an exemplary embodiment, the mRNA encodes from 5′ to 3′: a signal peptide, an Fc domain, a linker, and a RBD area, wherein the mRNA does not encode for a hinge.

In an exemplary embodiment, the mRNA encodes from 5′ to 3′: a signal peptide, a first RBD, a first linker, a hinge region, an Fc domain, a second linker, and a second RBD. In an exemplary embodiment, the mRNA encodes from 5′ to 3′: a signal peptide, a first RBD, a first linker, an Fc domain, a second linker, and a second RBD, wherein the mRNA does not encode for a hinge. In an exemplary embodiment, the first coronavirus spike protein RBD and the second coronavirus spike protein RBD are the same. In an exemplary embodiment, the first coronavirus spike protein RBD and the second coronavirus spike protein RBD are different. In an exemplary embodiment, the mRNA encodes from 5′ to 3′: a signal peptide, a first coronavirus spike protein RBD, a first linker encoded by a ribonucleotide sequence comprising SEQ ID NO: 33, 34, 35, 36, 37, 38, 39, 40, 41, 42, 43, 44, 45, 46, or 47 (D3-D17), a hinge encoded by a ribonucleotide sequence comprising SEQ ID NO: 81, 91, or 96 (H1, H11, H16), a Fc domain encoded by a ribonucleotide sequence comprising SEQ ID NO: 111, 115, 117, 122, or 129 (F1, F5, F7, F12, F19), a second linker encoded by a ribonucleotide sequence comprising SEQ ID NO: 57, 58, 59, 60, 61, 62, 63, 64, 65, 67 (D27-D37), and the second coronavirus spike protein RBD.

In an exemplary embodiment, the mRNA encodes from 5′ to 3′: a signal peptide, a first RBD area, a first linker, a hinge region, an Fc domain, a second linker, and a second RBD area. In an exemplary embodiment, the mRNA encodes from 5′ to 3′: a signal peptide, a first RBD area, a first linker, an Fc domain, a second linker, and a second RBD area, wherein the mRNA does not encode for a hinge. In an exemplary embodiment, the first coronavirus spike protein RBD area and the second coronavirus spike protein RBD area are the same. In an exemplary embodiment, the first coronavirus spike protein RBD area and the second coronavirus spike protein RBD area are different. In an exemplary embodiment, the mRNA encodes from 5′ to 3′: a signal peptide, a first coronavirus spike protein RBD area, a first linker encoded by a ribonucleotide sequence comprising SEQ ID NO: 33, 34, 35, 36, 37, 38, 39, 40, 41, 42, 43, 44, 45, 46, or 47 (D3-D17), a hinge encoded by a ribonucleotide sequence comprising SEQ ID NO: 81, 91, or 96 (H1, H11, H16), a Fc domain encoded by a ribonucleotide sequence comprising SEQ ID NO: 111, 115, 117, 122, or 129 (F1, F5, F7, F12, F19), a second linker encoded by a ribonucleotide sequence comprising SEQ ID NO: 57, 58, 59, 60, 61, 62, 63, 64, 65, 67 (D27-D37), and the second coronavirus spike protein RBD area.

In an exemplary embodiment, the mRNA encodes from 5′ to 3′: a signal peptide, a first coronavirus spike protein RBD which is a SARS-CoV-2 delta spike protein RBD or a SARS-CoV-2 omicron BA.2 spike protein RBD, a first linker encoded by a ribonucleotide sequence comprising SEQ ID NO: 33, 34, 35, 36, 37, 38, 39, 40, 41, 42, 43, 44, 45, 46, or 47 (D3-D17), a hinge encoded by a ribonucleotide sequence comprising SEQ ID NO: 81, 91, or 96 (H1, H11, H16), a Fc domain encoded by a ribonucleotide sequence comprising SEQ ID NO: 111, 115, 117, 122, or 129 (F1, F5, F7, F12, F19), a second linker encoded by a ribonucleotide sequence comprising SEQ ID NO: 57, 58, 59, 60, 61, 62, 63, 64, 65, 67 (D27-D37), and a second coronavirus spike protein RBD which is a SARS-CoV-2 delta spike protein RBD or a SARS-CoV-2 omicron BA.2 spike protein RBD. In an exemplary embodiment, the mRNA encodes from 5′ to 3′: a signal peptide, a first coronavirus spike protein RBD which is a SARS-CoV-2 delta spike protein RBD, a first linker encoded by a ribonucleotide sequence comprising SEQ ID NO: 33, 34, 35, 36, 37, 38, 39, 40, 41, 42, 43, 44, 45, 46, or 47 (D3-D17), a hinge encoded by a ribonucleotide sequence comprising SEQ ID NO: 81, 91, or 96 (H1, H11, H16), a Fc domain encoded by a ribonucleotide sequence comprising SEQ ID NO: 111, 115, 117, 122, or 129 (F1, F5, F7, F12, F19), a second linker encoded by a ribonucleotide sequence comprising SEQ ID NO: 57, 58, 59, 60, 61, 62, 63, 64, 65, 67 (D27-D37), and a second coronavirus spike protein RBD which is a SARS-CoV-2 omicron BA.2 spike protein RBD. In an exemplary embodiment, the mRNA encodes from 5′ to 3′: a signal peptide, a first coronavirus spike protein RBD encoded by a ribonucleotide sequence comprising SEQ ID NO: 154, 159, 162, 164, 165, 180, or 192 (B4, B9, B12, B14, B15, B30, or B42), a first linker encoded by a ribonucleotide sequence comprising SEQ ID NO: 33, 34, 35, 36, 37, 38, 39, 40, 41, 42, 43, 44, 45, 46, or 47 (D3-D17), a hinge encoded by a ribonucleotide sequence comprising SEQ ID NO: 81, 91, or 96 (H1, H11, H16), a Fc domain encoded by a ribonucleotide sequence comprising SEQ ID NO: 111, 115, 117, 122, or 129 (F1, F5, F7, F12, F19), a second linker encoded by a ribonucleotide sequence comprising SEQ ID NO: 57, 58, 59, 60, 61, 62, 63, 64, 65, 67 (D27-D37), and a second coronavirus spike protein RBD encoded by a ribonucleotide sequence comprising SEQ ID NO: 151, 154, 166, 167, 168, 169, 170, 171, 172, 173, 175, 181, or 193 (B1, B4, B16, B17, B18, B19, B20, B21, B22, B23, B25, B31, or B43). In an exemplary embodiment, the mRNA encodes from 5′ to 3′: a signal peptide, a first coronavirus spike protein RBD, a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), and a second coronavirus spike protein RBD. In an exemplary embodiment, the mRNA encodes from 5′ to 3′: a signal peptide, a first coronavirus spike protein RBD which is a SARS-CoV-2 delta spike protein RBD or a SARS-CoV-2 omicron BA.2 spike protein RBD, a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), and a second coronavirus spike protein receptor binding domain which is a SARS-CoV-2 delta spike protein RBD or a SARS-CoV-2 omicron BA.2 spike protein RBD. In an exemplary embodiment, the mRNA encodes from 5′ to 3′: a signal peptide, a first coronavirus spike protein RBD is a SARS-CoV-2 delta spike protein RBD, a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), and a second coronavirus spike protein RBD which is a SARS-CoV-2 omicron BA.2 spike protein RBD. In an exemplary embodiment, the mRNA encodes from 5′ to 3′: a signal peptide, a first coronavirus spike protein RBD, a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), a the second coronavirus spike protein RBD encoded by a ribonucleotide sequence comprising SEQ ID NO: 151, 166, 167, 169, 170, 172, 173, or 193 (B1, B16, B17, B19, B20, B22, B23, or B43). In an exemplary embodiment, the mRNA encodes from 5′ to 3′: a signal peptide, a first coronavirus spike protein RBD encoded by a ribonucleotide sequence comprising SEQ ID NO: 154 or 162 (B4, B12), a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), and a second coronavirus spike protein RBD encoded by a ribonucleotide sequence comprising SEQ ID NO: 151, 166, 167, 169, 170, 172, 173, or 193 (B1, B16, B17, B19, B20, B22, B23, or B43). In an exemplary embodiment, the mRNA sequence is as set forth in one of SEQ ID NOs: 331-372. In an exemplary embodiment, the monomeric polypeptide chain is as set forth in one of SEQ ID NOs: 631-672. In an exemplary embodiment, the monomeric polypeptide chain forms an antigenic protein capable of eliciting in a subject an immune response to a coronavirus.

In an exemplary embodiment, the mRNA encodes from 5′ to 3′: a signal peptide, a first coronavirus spike protein RBD area which is a SARS-CoV-2 delta spike protein RBD area or a SARS-CoV-2 omicron BA.2 spike protein RBD area, a first linker encoded by a ribonucleotide sequence comprising SEQ ID NO: 33, 34, 35, 36, 37, 38, 39, 40, 41, 42, 43, 44, 45, 46, or 47 (D3-D17), a hinge encoded by a ribonucleotide sequence comprising SEQ ID NO: 81, 91, or 96 (H1, H11, H16), a Fc domain encoded by a ribonucleotide sequence comprising SEQ ID NO: 111, 115, 117, 122, or 129 (F1, F5, F7, F12, F19), a second linker encoded by a ribonucleotide sequence comprising SEQ ID NO: 57, 58, 59, 60, 61, 62, 63, 64, 65, 67 (D27-D37), and a second coronavirus spike protein RBD area which is a SARS-CoV-2 delta spike protein RBD area or a SARS-CoV-2 omicron BA.2 spike protein RBD area. In an exemplary embodiment, the mRNA encodes from 5′ to 3′: a signal peptide, a first coronavirus spike protein RBD area which is a SARS-CoV-2 delta spike protein RBD area, a first linker encoded by a ribonucleotide sequence comprising SEQ ID NO: 33, 34, 35, 36, 37, 38, 39, 40, 41, 42, 43, 44, 45, 46, or 47 (D3-D17), a hinge encoded by a ribonucleotide sequence comprising SEQ ID NO: 81, 91, or 96 (H1, H11, H16), a Fc domain encoded by a ribonucleotide sequence comprising SEQ ID NO: 111, 115, 117, 122, or 129 (F1, F5, F7, F12, F19), a second linker encoded by a ribonucleotide sequence comprising SEQ ID NO: 57, 58, 59, 60, 61, 62, 63, 64, 65, 67 (D27-D37), and a second coronavirus spike protein RBD area which is a SARS-CoV-2 omicron BA.2 spike protein RBD area. In an exemplary embodiment, the mRNA encodes from 5′ to 3′: a signal peptide, a first coronavirus spike protein RBD area encoded by a ribonucleotide sequence comprising SEQ ID NO: 154, 159, 162, 164, 165, 180, or 192 (B4, B9, B12, B14, B15, B30, or B42), a first linker encoded by a ribonucleotide sequence comprising SEQ ID NO: 33, 34, 35, 36, 37, 38, 39, 40, 41, 42, 43, 44, 45, 46, or 47 (D3-D17), a hinge encoded by a ribonucleotide sequence comprising SEQ ID NO: 81, 91, or 96 (H1, H11, H16), a Fc domain encoded by a ribonucleotide sequence comprising SEQ ID NO: 111, 115, 117, 122, or 129 (F1, F5, F7, F12, F19), a second linker encoded by a ribonucleotide sequence comprising SEQ ID NO: 57, 58, 59, 60, 61, 62, 63, 64, 65, 67 (D27-D37), and a second coronavirus spike protein RBD area encoded by a ribonucleotide sequence comprising SEQ ID NO: 151, 154, 166, 167, 168, 169, 170, 171, 172, 173, 175, 181, or 193 (B1, B4, B16, B17, B18, B19, B20, B21, B22, B23, B25, B31, or B43). In an exemplary embodiment, the mRNA encodes from 5′ to 3′: a signal peptide, a first coronavirus spike protein RBD area, a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), and a second coronavirus spike protein RBD area. In an exemplary embodiment, the mRNA encodes from 5′ to 3′: a signal peptide, a first coronavirus spike protein RBD area which is a SARS-CoV-2 delta spike protein RBD area or a SARS-CoV-2 omicron BA.2 spike protein RBD area, a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), and a second coronavirus spike protein receptor binding domain which is a SARS-CoV-2 delta spike protein RBD area or a SARS-CoV-2 omicron BA.2 spike protein RBD area. In an exemplary embodiment, the mRNA encodes from 5′ to 3′: a signal peptide, a first coronavirus spike protein RBD area is a SARS-CoV-2 delta spike protein RBD area, a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), and a second coronavirus spike protein RBD area which is a SARS-CoV-2 omicron BA.2 spike protein RBD area. In an exemplary embodiment, the mRNA encodes from 5′ to 3′: a signal peptide, a first coronavirus spike protein RBD area, a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), a the second coronavirus spike protein RBD area encoded by a ribonucleotide sequence comprising SEQ ID NO: 151, 166, 167, 169, 170, 172, 173, or 193 (B1, B16, B17, B19, B20, B22, B23, or B43). In an exemplary embodiment, the mRNA encodes from 5′ to 3′: a signal peptide, a first coronavirus spike protein RBD area encoded by a ribonucleotide sequence comprising SEQ ID NO: 154 or 162 (B4, B12), a first linker encoded by a ribonucleotide sequence comprising SEQ ID NO: 33 (D3), a hinge encoded by a ribonucleotide sequence comprising SEQ ID NO: 81 (H1), a Fc domain encoded by a ribonucleotide sequence comprising SEQ ID NO: 115 (F5), a second linker encoded by a ribonucleotide sequence comprising SEQ ID NO: 57 (D27), and a second coronavirus spike protein RBD area encoded by a ribonucleotide sequence comprising SEQ ID NO: 151, 166, 167, 169, 170, 172, 173, or 193 (B1, B16, B17, B19, B20, B22, B23, or B43).

### c) Proteins

The present disclosure provides an antigenic protein for eliciting in a subject an immune response to a coronavirus, such as an immune response to a coronavirus spike (S) protein. In some embodiments, the antigenic protein is produced by the subject's cellular machinery by providing to the subject an mRNA of the present disclosure that encodes the antigenic protein. In an exemplary embodiment, the antigenic protein is a monomeric polypeptide chain. In an exemplary embodiment, the antigenic protein comprises two monomeric polypeptide chains, which may be the same (identical) or different. In an exemplary embodiment, the antigenic protein has two monomeric polypeptide chains, which may be the same (identical) or different. Each monomeric polypeptide chain comprises one or more receptor binding domains, one or more linker domains, and an Fc domain. In some embodiments, the monomeric polypeptide chain further comprises an antibody hinge region. The antigenic proteins of the disclosure are made as monomeric polypeptide chains comprising at least one receptor binding domain and an Fc domain. In an exemplary embodiment, the antigenic protein further comprises a hinge. The at least one receptor binding domain and the Fc domain are connected by at least one linker. The antigenic proteins comprise two monomeric polypeptide chains. When the two monomeric polypeptide chains are identical, the chains may assemble into homodimeric complexes. When the two monomeric polypeptide chains are different, the chains may assemble into homodimeric and/or heterodimeric complexes. Formation of dimeric complexes is facilitated by the presence of the Fc domain. In some embodiments, formation of dimeric complexes is facilitated by binding of the CH3 domain of one monomeric polypeptide chain to the CH3 domain of the other polypeptide chain and/or by formation of disulfide bonds between the hinge of one monomeric polypeptide chain to the hinge of the other polypeptide chain. In some embodiments, cysteine residues in the hinge form interchain disulfide bonds between the two monomeric polypeptide chains.

In some embodiments, the antigenic proteins of the disclosure comprise at least one linker operatively linking the RBD and the Fc domain. In some embodiments, the RBD may be linked to the N-terminus of the linker, which is in turn linked to the N-terminus of the Fc domain. In some embodiments, the RBD may be linked to the C-terminus of the linker, which is in turn linked to the C-terminus of the Fc domain. In some embodiments, a first RBD may be linked to the N-terminus of a first linker, which is in turn linked to the N-terminus of the Fc domain, and a second RBD may be linked to the C-terminus of a second linker, which is in turn linked to the C-terminus of the Fc domain.

In some embodiments, the antigenic proteins of the disclosure comprise at least one linker operatively linking the RBD area and the Fc domain. In some embodiments, the RBD area may be linked to the N-terminus of the linker, which is in turn linked to the N-terminus of the Fc domain. In some embodiments, the RBD area may be linked to the C-terminus of the linker, which is in turn linked to the C-terminus of the Fc domain. In some embodiments, a first RBD area may be linked to the N-terminus of a first linker, which is in turn linked to the N-terminus of the Fc domain, and a second RBD area may be linked to the C-terminus of a second linker, which is in turn linked to the C-terminus of the Fc domain.

In some embodiments, the amino acid linker comprises a glycine sequence (e.g., two or more glycine residues) with one or more serine residues positioned in between, or C- or N-terminus, to the glycine residues. In certain embodiments, the linker comprises GGGGSGGGGS (SEQ ID NO: 513), GGGGSGGGGSGGGGS (SEQ ID NO: 514), or GGGGSGGGGSGGGGSGGGGS (SEQ ID NO: 515).

In some embodiments, the antigenic proteins of the disclosure comprise from N-terminus to C-terminus: a RBD, a linker, a hinge, and an Fc domain. In some embodiments, the antigenic proteins of the disclosure comprise from N-terminus to C-terminus: a RBD, a linker, and an Fc domain, wherein the antigenic protein does not comprise a hinge. In some embodiments, the antigenic proteins of the disclosure comprise from N-terminus to C-terminus: a hinge, an Fc domain, a linker, and a RBD. In some embodiments, the antigenic proteins of the disclosure comprise from N-terminus to C-terminus: an Fc domain, a linker, and a RBD, wherein the antigenic protein does not comprise a hinge. In some embodiments, the antigenic proteins of the disclosure comprise from N-terminus to C-terminus: a first RBD, a first linker, a hinge, an Fc domain, a second linker, and a second RBD. In some embodiments, the antigenic proteins of the disclosure comprise from N-terminus to C-terminus: a first RBD, a first linker, an Fc domain, a second linker, and a second RBD, wherein the antigenic protein does not comprise a hinge.

In some embodiments, the antigenic proteins of the disclosure comprise from N-terminus to C-terminus: a RBD area, a linker, a hinge, and an Fc domain. In some embodiments, the antigenic proteins of the disclosure comprise from N-terminus to C-terminus: a RBD area, a linker, and an Fc domain, wherein the antigenic protein does not comprise a hinge. In some embodiments, the antigenic proteins of the disclosure comprise from N-terminus to C-terminus: a hinge, an Fc domain, a linker, and a RBD area. In some embodiments, the antigenic proteins of the disclosure comprise from N-terminus to C-terminus: an Fc domain, a linker, and a RBD area, wherein the antigenic protein does not comprise a hinge. In some embodiments, the antigenic proteins of the disclosure comprise from N-terminus to C-terminus: a first RBD area, a first linker, a hinge, an Fc domain, a second linker, and a second RBD area. In some embodiments, the antigenic proteins of the disclosure comprise from N-terminus to C-terminus: a first RBD area, a first linker, an Fc domain, a second linker, and a second RBD area, wherein the antigenic protein does not comprise a hinge.

In some embodiments, the antigenic proteins of the disclosure comprise from N-terminus to C-terminus: a signal peptide, a RBD, a linker, a hinge, and an Fc domain. In some embodiments, the antigenic proteins of the disclosure comprise from N-terminus to C-terminus: a signal peptide, a RBD, a linker, and an Fc domain, wherein the antigenic protein does not comprise a hinge. In some embodiments, the antigenic proteins of the disclosure comprise from N-terminus to C-terminus: a signal peptide, a hinge, an Fc domain, a linker, and a RBD. In some embodiments, the antigenic proteins of the disclosure comprise from N-terminus to C-terminus: a signal peptide, a Fc domain, a linker, and a RBD, wherein the antigenic protein does not comprise a hinge. In some embodiments, the antigenic proteins of the disclosure comprise from N-terminus to C-terminus: a signal peptide, a first RBD, a first linker, a hinge, an Fc domain, a second linker, and a second RBD. In some embodiments, the antigenic proteins of the disclosure comprise from N-terminus to C-terminus: a signal peptide, a first RBD, a first linker, an Fc domain, a second linker, and a second RBD, wherein the antigenic protein does not comprise a hinge.

In some embodiments, the antigenic proteins of the disclosure comprise from N-terminus to C-terminus: a signal peptide, a RBD area, a linker, a hinge, and an Fc domain. In some embodiments, the antigenic proteins of the disclosure comprise from N-terminus to C-terminus: a signal peptide, a RBD area, a linker, and an Fc domain, wherein the antigenic protein does not comprise a hinge. In some embodiments, the antigenic proteins of the disclosure comprise from N-terminus to C-terminus: a signal peptide, a hinge, an Fc domain, a linker, and a RBD area. In some embodiments, the antigenic proteins of the disclosure comprise from N-terminus to C-terminus: a signal peptide, a Fc domain, a linker, and a RBD area, wherein the antigenic protein does not comprise a hinge. In some embodiments, the antigenic proteins of the disclosure comprise from N-terminus to C-terminus: a signal peptide, a first RBD area, a first linker, a hinge, an Fc domain, a second linker, and a second RBD area. In some embodiments, the antigenic proteins of the disclosure comprise from N-terminus to C-terminus: a signal peptide, a first RBD area, a first linker, an Fc domain, a second linker, and a second RBD area, wherein the antigenic protein does not comprise a hinge.

In some embodiments, the RBD is a RBD from a coronavirus spike (S) protein. In some embodiments, the RBD is a RBD from a SARS-CoV-2 S protein. The SARS-CoV-2 S protein consists of a signal peptide (amino acids 1-13) located at the N-terminus, the S1 subunit (amino acids 14-685), and the S2 subunit (amino acids 686-1273). The S1 and S2 subunits are responsible for receptor binding and membrane fusion, respectively. In the S1 subunit, there is an N-terminus domain (amino acids 14-305) and a receptor-binding domain (RBD, amino acids 319-541). In the S2 subunit, contains the fusion peptide (FP) (amino acids 788-806), heptapeptide repeat sequence 1 (HR1) (amino acids 912-984), HR2 (amino acids 1163-1213), TM domain (amino acids 1213-1237), and cytoplasm domain (amino acids 1237-1273). In some embodiments, the RBD area comprises a RBD from a coronavirus spike (S) protein. In some embodiments, the RBD area is a RBD area from a SARS-CoV-2 S protein.

In some embodiments, the receptor binding domain comprises amino acids 167-541 of a coronavirus S protein, such as a SARS-CoV-2 S protein; amino acids 311-532 of a coronavirus S protein, such as a SARS-CoV-2 S protein; amino acids 319-537 of a coronavirus S protein, such as a SARS-CoV-2 S protein; amino acids 319-541 of a coronavirus S protein, such as a SARS-CoV-2 S protein; or amino acids 333-529 of a coronavirus S protein, such as a SARS-CoV-2 S protein.

In some embodiments, the receptor binding domain is receptor binding domain from a SARS-CoV-2 S protein variant. In some embodiments, the receptor binding domain is receptor binding domain from a SARS-CoV-2 S protein alpha variant, beta variant, delta variant, or gamma variant. In some embodiments, the receptor binding domain is receptor binding domain from a SARS-CoV-2 S protein delta variant.

In some embodiments, the receptor binding domain comprises amino acids 167-541 of a SARS-CoV-2 S protein variant, such as a SARS-CoV-2 S protein delta variant; amino acids 311-532 of a SARS-CoV-2 S protein variant, such as a SARS-CoV-2 S protein delta variant; amino acids 319-537 of a SARS-CoV-2 S protein variant, such as a SARS-CoV-2 S protein delta variant; amino acids 319-541 of a SARS-CoV-2 S protein variant, such as a SARS-CoV-2 S protein delta variant; or amino acids 333-529 of a SARS-CoV-2 S protein variant, such as a SARS-CoV-2 S protein delta variant.

In some embodiments, the antigenic protein comprises first and second receptor binding domains. In some embodiments, both the first and second receptor binding proteins are a receptor binding domain from a wild type SARS-CoV-2 S protein. In some embodiments, both the first and second receptor binding proteins are a receptor binding domain from a SARS-CoV-2 S protein delta variant. In some embodiments, the first receptor binding protein is a receptor binding domain from a wild type SARS-CoV-2 S protein and the second receptor binding protein is a receptor binding protein from a SARS-CoV-2 S protein delta variant. In some embodiments, the first receptor binding protein is a receptor binding domain from a wild type SARS-CoV-2 S protein and is positioned N-terminus to the Fc domain, and the second receptor binding protein is a receptor binding protein from a SARS-CoV-2 S protein delta variant and is positioned C-terminus to the Fc domain. In some embodiments, the first receptor binding protein is a receptor binding domain from a wild type SARS-CoV-2 S protein and is positioned C-terminus to the Fc domain, and the second receptor binding protein is a receptor binding protein from a SARS-CoV-2 S protein delta variant and is positioned N-terminus to the Fc domain.

In some embodiments, the antigenic protein comprises a signal peptide. In some embodiments, the signal peptide is a signal peptide from a SARS-CoV-2 S protein, a signal peptide from an influenza hemagglutinin (HA) protein, a signal peptide from a vesicular stomatitis virus G (VSV-G) protein, a signal peptide from an albumin (e.g., a human serum albumin (HSA)) protein, or a signal peptide from a human IgG2 heavy chain. In some embodiments, the antigenic protein comprises a signal peptide from a SARS-CoV-2 S protein. In some embodiments, the antigenic protein comprises a signal peptide from a SARS-CoV-2 S protein variant. In some embodiments, the antigenic protein comprises a signal peptide from a SARS-CoV-2 S protein alpha variant, beta variant, delta variant, or gamma variant. In some embodiments, the antigenic protein comprises a signal peptide from a SARS-CoV-2 S protein delta variant. In some embodiments, the antigenic protein lacks a signal peptide.

In an exemplary embodiment, the monomeric polypeptide chain comprises a first coronavirus spike protein receptor binding domain, a first linker, a hinge, a Fc domain, a second linker, and a second coronavirus spike protein receptor binding domain. In an exemplary embodiment, the monomeric polypeptide chain comprises, from N-terminus to C-terminus, the first coronavirus spike protein receptor binding domain, the first linker, the hinge, the Fc domain, the second linker, and the second coronavirus spike protein receptor binding domain.

In an exemplary embodiment, the first linker and the second linker of the monomeric polypeptide chain each independently comprise SEQ ID NO: 511, 512, 513, 514, 515, or 516 (K1-K6). In an exemplary embodiment, the first linker of the monomeric polypeptide chain comprises SEQ ID NO: 513 (K3). In an exemplary embodiment, the second linker of the monomeric polypeptide chain comprises SEQ ID NO: 515 (K5).

In an exemplary embodiment, the hinge of the monomeric polypeptide chain comprises SEQ ID NO: 521, 522, 523, 524, 525, 526, or 527. (I1-I7) In an exemplary embodiment, the hinge of the monomeric polypeptide chain comprises SEQ ID NO: 521. (I1)

In an exemplary embodiment, the Fc domain of the monomeric polypeptide chain comprises SEQ ID NO: 541, 542, 543, 544, 545, 546, 547, or 548 (G1-G8). In an exemplary embodiment, the Fc domain of the monomeric polypeptide chain comprises SEQ ID NO: 541, 545, or 547 (G1, G5, G7). In an exemplary embodiment, the Fc domain of the monomeric polypeptide chain comprises SEQ ID NO: 545 (G5).

In an exemplary embodiment, the first coronavirus spike protein receptor binding domain of the monomeric polypeptide chain is a SARS-CoV-2 spike protein receptor binding domain. In an exemplary embodiment, the first coronavirus spike protein receptor binding domain of the monomeric polypeptide chain is a SARS-CoV-2 wild-type spike protein receptor binding domain, a SARS-CoV-2 beta spike protein receptor binding domain, a SARS-CoV-2 lambda spike protein receptor binding domain, a SARS-CoV-2 delta spike protein receptor binding domain, a SARS-CoV-2 mu spike protein receptor binding domain, a SARS-CoV-2 omicron BA.1 spike protein receptor binding domain, a SARS-CoV-2 omicron BA.1.1 spike protein receptor binding domain, or a SARS-CoV-2 omicron BA.2 spike protein receptor binding domain. In an exemplary embodiment, the first coronavirus spike protein receptor binding domain of the monomeric polypeptide chain is a SARS-CoV-2 delta spike protein receptor binding domain, a SARS-CoV-2 omicron BA.1 spike protein receptor binding domain, a SARS-CoV-2 omicron BA.1.1 spike protein receptor binding domain, or a SARS-CoV-2 omicron BA.2 spike protein receptor binding domain. In an exemplary embodiment, the second coronavirus spike protein receptor binding domain of the monomeric polypeptide chain is a SARS-CoV-2 spike protein receptor binding domain. In an exemplary embodiment, the second coronavirus spike protein receptor binding domain of the monomeric polypeptide chain is a SARS-CoV-2 delta spike protein receptor binding domain, a SARS-CoV-2 omicron BA. 1 spike protein receptor binding domain, a SARS-CoV-2 omicron BA.1.1 spike protein receptor binding domain, or a SARS-CoV-2 omicron BA.2 spike protein receptor binding domain. In an exemplary embodiment, the first coronavirus spike protein receptor binding domain and the second coronavirus spike protein receptor binding domain of the monomeric polypeptide chain each independently comprise SEQ ID NO: 561, 562, 563, 564, 565, 566, 567, 568, 569, 570, 571, 572, 573, 574, 575, 576, 577, 578, 579, 580, or 581 (R1-R21). In an exemplary embodiment, the first coronavirus spike protein receptor binding domain of the monomeric polypeptide chain comprises SEQ ID NO: 564, 569, 571, or 572 (R4, R9, R11, or R12). In an exemplary embodiment, the first coronavirus spike protein receptor binding domain of the monomeric polypeptide chain comprises SEQ ID NO: 564 or 572 (R4 or R12). In an exemplary embodiment, the first coronavirus spike protein receptor binding domain of the monomeric polypeptide chain comprises SEQ ID NO: 564 (R4). In an exemplary embodiment, the second coronavirus spike protein receptor binding domain of the monomeric polypeptide chain comprises SEQ ID NO: 561, 564, 572, 573, 574, 575, 576, 577, 578, or 579 (R1, R4, R12, R13, R14, R15, R16, R17, R18, or R19). In an exemplary embodiment, the second coronavirus spike protein receptor binding domain of the monomeric polypeptide chain comprises SEQ ID NO: 561, 564, 572, 573, 575, 576, 578, or 579 (R1, R4, R12, R13, R15, R16, R18, or R19). In an exemplary embodiment, the second coronavirus spike protein receptor binding domain of the monomeric polypeptide chain comprises SEQ ID NO: 579 (R19). In an exemplary embodiment, the monomeric polypeptide chain is as set forth in one of SEQ ID NOs: 631-672. In an exemplary embodiment, the monomeric polypeptide chain forms an antigenic protein capable of eliciting in a subject an immune response to a coronavirus.

In an exemplary embodiment, the antigenic protein comprises two monomeric polypeptide chains, wherein each monomeric polypeptide chain is as described herein, wherein the antigenic protein is capable of eliciting in a subject an immune response to a coronavirus. In an exemplary embodiment, the monomeric polypeptide chain comprises a hinge, and cysteine residues in the hinge form interchain disulfide bonds between the two monomeric polypeptide chains. In an exemplary embodiment, the two monomeric polypeptide chains have the same sequence. In an exemplary embodiment, the two monomeric polypeptide chains have different sequences. In an exemplary embodiment, at least one of the monomeric polypeptide chains is according to SEQ ID NOs: 631-672.

### d) Vaccine

The disclosure provides compositions comprising the mRNA of the present disclosure. In some embodiments, the mRNA of the present disclosure is formulated in a lipid nanoparticle. Suitable lipid nanoparticle formulations are described, for example, in U.S. Pat. No. 10,702,600.

The disclosure provides a vaccine comprising the mRNA and a pharmaceutically acceptable carrier. In some embodiments, the vaccine comprises the mRNA formulated in a lipid nanoparticle. Suitable lipid nanoparticle formulations are described, for example, in U.S. Pat. No. 10,702,600.

### e) Methods of Use

The disclosure provides methods of eliciting an immune response in a subject in need thereof. The methods comprise providing to the subject the vaccine of the present disclosure. In some embodiments, the immune response is an antigen-specific immune response. In some embodiments, an antigen-specific immune response comprises a T cell response or a B cell response.

In some embodiments, the immune response is assessed by determining antibody titer in the subject for an antibody that binds to a coronavirus antigenic polypeptide.

In some embodiments, the method of eliciting an immune response in a subject comprises administering to the subject a single dose (no booster dose) of the vaccine of the present disclosure. In some embodiments, the method of eliciting an immune response in a subject comprises administering to the subject an initial dose and one or more booster doses of the vaccine of the present disclosure.

The disclosure provides methods of preventing and/or treating a coronavirus infection in a subject in need thereof. The disclosure also provides methods for preventing the occurrence of COVID-19 in a subject in need thereof. The methods comprise administering to the subject a vaccine comprising the mRNA of the present disclosure.

As used herein, “preventing” or “treating” a disease, disorder, or condition in a subject means reducing at least one symptom of the disease, disorder, or condition by administering an mRNA of the present disclosure or pharmaceutical preparation thereof to the subject. By “subject” is meant either a human or non-human animal (e.g., a mammal).

In some embodiments, the coronavirus infection is a severe acute respiratory syndrome (SARS)-CoV (SARS-CoV) infection, a Middle East respiratory syndrome (MERS)-CoV (MERS-CoV) infection, or a SARS-CoV-2 infection.

In some embodiments, a vaccine of the present disclosure is administered to a subject by intradermal or intramuscular injection.

### f) Additional Antigens and Methods of Use

The disclosure also provides mRNAs, antigenic proteins encoded by the mRNAs, compositions of the mRNAs, and vaccines comprising the mRNAs that are suitable for preventing and/or treating additional diseases or disorders. Exemplary diseases and disorders that may be prevented and/or treated by the methods disclosed herein include, but are not limited to, the diseases and disorders listed in the following table. Suitable constructs for preventing and/or treating such diseases and disorders comprise an antigen protein as listed in the following table, or a fragment thereof, in place of the receptor binding domains as disclosed herein.

| Type of Disease | | |
|---|---|---|
| or Disorder | Disease or Disorder | Antigen Protein |
| Viral infection | RSV | F protein |
| Viral infection | Zika virus | envelope domain III |
| Viral infection | Henipavirus | G proteins |
| Viral infection | Dengue | envelope domain III |
| Viral infection | Classical swine fever | E2 |
| Viral infection | HCV | E1/E2 |
| Viral infection | HIV | gp120 |
| Viral infection | Flu | HA |
| Viral infection | SARS-CoV-2 | S1 |
| Viral infection | SARS-CoV | RBD |
| Viral infection | HPV | E6/E7 |
| Viral infection | EBV | gp350 |
| Cancer | neuroblastoma | 47-LDA mimotope |
| Cancer | lymphoma | NKG2D |
| Cancer | bladder cancer | hCG-β |
| Cancer | Colorectal Cancer | GA733 |
| Cancer | triple-negative breast | HAGE-derived sequence |
| | cancer | |
| Mycobacterium | Tuberculosis | ESAT-6/CFP-10 |
| tuberculosis | | |
| infection | | |
| Autoimmune disease | arthritis | single-chain peptide |
| Autoimmune disease | autoimmune | N-terminus epitope of |
| | encephalomyelitis | myelin basic protein |
| Allergy | cat-induced allergy | Fel d1 |
| Allergy | allergic | DARPin |

### EXAMPLES

### General Experimental Methods

The following general procedures were used.

### Transfection

HEK293T cells were seeded in 12-well plates at 350,000 cells/well with 5% FBS and 1% antibiotics. Cells were transfected by Lipofectamine 3000 (Thermo Fisher Scientific) eighteen hours later with plasmids individually. After 40 hours cultivation, the supernatant and cell lysate were collected and tested by ELISA or WB.

### WB

36 ul cell lysate and 9 ul 5× loading buffer was mixed and boiled at 100° C. for 10 min. 45 ul mixture was loaded into the wells of the 10% SDS-PAGE gel, along with a molecular weight marker. The gel was run for 1-2 h at 100 V. PVDF was activated with methanol for 1 min and rinsed with transfer buffer before preparing the stack. Proteins were transferred to the membrane for 1 h at 100V.

The membrane was blocked for 1 h at room temperature or overnight at 4° C. using blocking buffer. The membrane was then incubated with 1:1000 dilution of RBD monoclonal antibody in 3% BSA for 1 h at room temperature. Next, the membrane was washed in three washes of Tris-buffered saline and Tween 20 (TBST, 5 min each). The membrane was then incubated with 1:10000 dilutions of HRP-conjugated secondary antibody in 3% BSA for 1 h at room temperature and washed in five washes of TBST (5 min each). Working Solution was prepared by mixing equal parts of the Stable Peroxide Solution and the Luminol/Enhancer Solution (Thermo SuperSignal™ West Pico PLUS Chemiluminescent Substrate). 0.1 mL Working Solution per cm² of membrane was used and incubated for 5 minutes. The blot was removed from Working Solution and excess reagent was drained. The blot was placed in clear plastic wrap and exposed to the imaging system.

### RBD Antigen Detection ELISA

First, the serum of mice or the supernatant of transfected cell lysate were all collected. The Sino SARS-CoV-2 (2019-nCOV) Spike RBD ELISA Kit was used to detect the RBD antigen. Briefly, each well was washed three times with wash buffer, and 100 μL of each serially diluted protein standard or test sample was added per well including a zero standard. The plate was covered/sealed and incubated for 2 hours at room temperature. The wash step was repeated and then 100 μL of Detection Antibody was added in working concentration to each well. The plate was covered/sealed and incubated for 1 hour at room temperature. The wash step was repeated. 200 μL of Substrate Solution was added to each well and the wells were incubated for 20 minutes at room temperature (protected from light). 50 μL of Stop Solution was added to each well. If color change did not appear uniform, the plate was gently tapped to ensure thorough mixing. The optical density of each well was determined within 20 minutes, using a microplate reader set to 450 nm.

### HDI Animal Model

The plasmids for HDI assay were constructed using pCDNA 3.4 vector. The total amount of a plasmid for one mouse was 8% of the body weight, the concentration used was 5 μg/ml and 3 mice were used for each group. 24 hours after HDI, 20 ul serum of each mouse were collected and the serum concentration of RBD-Fc fusion protein was detected by ELISA.

### LNP-mRNA

T7 DNA plasmids were synthesized and constructed. Fragments containing the T7 promotor sequence were amplified by PCR using primer pairs (F: TAATACGACTCACTATAG R-RBD: CTAGAAATTGACACATTTGTTTTTAACCAAATTAGTAG or F: TAATACGACTCACTATAG/R-Fc: CTAGCCCAGAGACAGGCTCAGGGACT). All these fragments were purified by Axygen Clean Up Kit and then added to the T7 IVT Kit reation system. The UTP in the IVT system was all replaced by the Pseudo-UTP and Cap1 was added at a final concentration of 4 mM. Two hours later after incubation at 37° C., 10 μl of 10× DNase I Buffer, and 2 μl of RNase-free DNase I were added, mixed and incubated at 37° C. for 15 minutes. RNA Cleanup Kit was used to purify the in vitro synthesized mRNA. Five units of the Poly(A) Polymerase was incubated with 1-10 μg RNA in a 20 μl reaction at 37° C. for 30 minutes (with 1× Reaction buffer and 1 mM ATP), resulting in a tail length of greater than 100 bases. Again the RNA Cleanup Kit was used to purify the tailed mRNA.

Lipid-nanoparticle (LNP) formulations were prepared using a modified procedure of a method previously described for siRNA (Ickenstein and Garidel, 2019). Briefly, lipids were dissolved in ethanol containing an ionizable lipid, 1,2-distearoyl-sn-glycero-3-phosphocholine (DSPC), cholesterol and PEG-lipid (with molar ratios of 50:10:38.5:1.5). The lipid mixture was combined with 20 mM citrate buffer (pH4.0) containing mRNA at a ratio of 1:2 through a PNI Spark machine. Formulations were then diafiltrated against 10× volume of PBS (pH 7.4) through 100 kD molecular weight cut-offs (Millipore) and concentrated to desired concentrations and stored at 4° C. until use. All formulations were tested for particle size, distribution, RNA concentration and encapsulation.

### Mouse Vaccination

Female Balb/c mice (6-8 week-old) were purchased and were randomly divided into treatment groups (n=3/group) and negative control group (2/group).

In some testings, mRNA-LNP or LNP was intramuscularly administrated into animals (15 ug/mouse). The orbital blood was collected at 6 h/30 h after administration, centrifuged at 5,000 g at 4° C. for 10 minutes. Sera were collected and stored at −80° C. for further testing. RBD expression level was determined by ELISA.

In other testing, the treatment groups were intramuscularly dosed with 100 μL LNP-mRNA. The negative control group was injected with an equal volume (100 μL) of lipid component at the same time.

Blood samples were taken from a thigh vein at 6, 30 hours and 3, 14, 21, 28 days after dosing. Blood samples were placed in an incubator at 37° C. for approximately 30 min, and then centrifuged in a precooled (0-4° C.) centrifuge to obtain serum samples which were stored at −20° C. for further testing.

### Competitive Inhibition Assay

Competitive inhibition assay was performed using SARS-CoV-2 pseudovirus and flat bottom clear, black polystyrene 96-well plates. Briefly, HEK-293T-ACE2 cells were seeded in a 96-well plate at 15,000 cells/well for 20 hours at 37° C. The culture medium of each well was discarded and cells were incubated with BSA, RBD-Fc (purified RBD Delta-Fc protein), recombinant RBD-Fc (rRBD-Fc), diluted serum or cell lysates for 1 hour at 37° C., followed by treatment with 1 ul/well of the Delta strain or 0.3 ul/well of the Wildtype strain SARS-CoV-2 pseudovirus for 2 hour at 37° C. The culture medium of each well was then discarded and 120 μl fresh culture medium was added to each well. Luciferase substrate was then added to plates followed by incubation in darkness at room temperature for 5 minutes and the detection of luminescence using Varioskan LUX (Thermo).

### RBD-ACE2 Binding ELISA

A multi-well plate was coated with 0.2 μg/well (2 μg/ml, 100 μl/well) Human ACE2, His Tag at 4° C. for overnight (or 16 hours). The protein was diluted in Coating Buffer (15 mmol/L Na₂CO₃, 35 mmol/L NaHCO₃, 7.7 mmol/L NaN₃, pH9.6). After washing for 4 times, the plate was inverted and allowed to sit on clean paper towels to ensure it was completely dried. The wells were blocked with 300 μl Blocking Buffer (2% BSA in Washing Buffer, pH7.4) per well at 37° C. for 1.5 hours. Washing was repeated and 100 μl SARS-CoV-2 S protein RBD-Fc or diluted serum was added to each well, followed by incubation at 37° C. for 1 hour. The sample was diluted in Sample Dilution Buffer (0.5% BSA in Washing Buffer, pH7.4). Washing was repeated and 100 μl Peroxidase AffiniPure Goat Anti-Human IgG, Fcγ fragment specific was added to each well, followed by incubation at 37° C. for 1 hour. The antibody was diluted 1:20000 in Antibody Dilution Buffer. Dilution Buffer: 0.5% BSA in Washing Buffer, pH 7.4. Washing was repeated and 200 μl Substrate Solution was added into each well, followed by incubation at 37° C. for 20 min. Light was avoided. Substrate Solution: 8 μl 3% H₂O₂ and 100 μl 10 mg/mL TMB in 10 mL Substrate Solution A (50 mmol/L Na₂HPO₄·12H₂O, 25 mmol/L Citric acid, pH 5.5). 50 μl 1 mol/L sulfuric acid was added to each well. OD was read at 450 nm, then OD450-Blank is the final OD Value. RBD antibody detection Elisa

ELISA plates (Corning) were coated overnight with 4 μg/ml of SARS-CoV-2 RBD recombinant protein in 0.05 M PBS, pH 9.6, and blocked in 3% skim milk in PBS at 37° C. for 1 h. 100 μl serum samples were diluted and added to each well and then incubated at 37° C. for 1 h. Plates were washed for 5 times by PBST and incubated with goat anti-mouse IgG-HRP antibodies at 37° C. for 1 h. The wash step was repeated and the plates were developed with 3,3′,5,5′-tetramethylbenzidine (TMB) substrate. Reactions were stopped with 3 M hydrochloric acid, and the absorbance was measured at 450 nm using a microplate reader (Thermo).

### Pseudovirus Neutralization Assay

Pseudovirus neutralization assay was performed using SARS-CoV-2 pseudovirus and flat bottom clear, black polystyrene 96-well plates (Corning 3603). 0.9 ul/well pseudovirus was incubated with twofold serially diluted mouse serum for 1 h at 37° C. The mixtures were then used to infect HEK-293-ACE2 cells seeded in 96-well plates. After 6 h incubation, the medium was replaced with DMEM containing 10% FBS, and the samples were incubated for an additional 24 h at 37° C. Luciferase activity was measured using Varioskan LUX (Thermo). The neutralization endpoint was defined as the fold-dilution of serum necessary for 50% inhibition of luciferase activity in comparison with virus control samples.

### Example 1: Construction of Polynucleotides

Polynucleotides of the disclosure were generated according to the following procedure.

First, T7-5′UTR and 3′UTR-poly(A) was synthesized as one fragment and cloned into PUC19 as an in vitro transcription (IVT) original vector. Then the IVT original vector was linearized by Nco I and Xho I. The three open reading frames (ORFs) were synthesized and constructed into the linearized IVT original vector by homologous recombination.

Specifically, RBD-linker-IgG4 Fc, IgG4 Fc-linker-RBD and RBD-linker-IgG4 Fc-linker-RBD were synthesized. RBD-linker-IgG4 Fc was amplified by F1/R-Fc primer pair, IgG4 Fc-linker-RBD and RBD-linker-IgG4 Fc-linker-RBD were amplified by F1/R-RBD primer pair respectively. The PCR product was designed to share a 15-nt homologous sequence (underlined) with the ends of the linearized IVT original template. An In-Fusion cloning mixture (Clontech) was used to assemble the PCR product and the linearized vector in vitro. The RBD-linker-IgG4 Fc, IgG4 Fc-linker-RBD and RBD-linker-IgG4 Fc-linker-RBD PCR products were individually inserted into the linearized IVT original vector.

The following primers were used, where uppercase letters represent the primer binding region, lowercase letters represent the homology arms:

Primer F1:
5′-ctcagagagaacccgccaccATGTTTGTTTTTCTTGTTT
TATTGCCACTAGTCTC-3′
Primer R-Fc:
5′-gcatgcagtaccagctcgagctaCTAGCCCAGAGACAGG
CTC-3′
Primer R-RBD:
5′-gcatgcagtaccagctcgagctaCTAGAAATTGACACAT
TTGTTTTTAACCAAATTAGTAG-3′

The nucleotide constructs described in Tables A to D were prepared.

TABLE A
Nucleotide Sequences of Control Constructs

| Ref No. | 5′UTR | Signal | -RBD | 3′UTR |
|---|---|---|---|---|
| PS1 | U1 | A1 | B4 | U4 |
| PS2 | U1 | A1 | B51 | U4 |
| PS3 | U1 | A15 | B1 | U4 |
| PS4 | U1 | A15 | B1 | U4 |

TABLE B
Nucleotide Sequences of N-Fusion Constructs

| Ref No. | 5′UTR | Signal | N′-RBD | | N′-Linker | Hinge | Fc | 3′UTR |
|---|---|---|---|---|---|---|---|---|
| PN8 | U1 | A1 | B4 | | — | H1 | F1 | U4 |
| PN9 | U1 | A1 | B4 | | D3 | H1 | F1 | U4 |
| PN10 | U1 | A1 | B4 | | D18 | H1 | F1 | U4 |
| PN11 | U1 | A1 | B4 | | D27 | H1 | F1 | U4 |
| PN12 | U1 | A1 | E1 | B4 | D3 | H1 | F1 | U4 |
| PN13 | U1 | A1 | B4 | | — | — | F1 | U4 |
| PN14 | U1 | A1 | B4 | | D3 | — | F1 | U4 |
| PN15 | U1 | A1 | B4 | | D18 | — | F1 | U4 |
| PN16 | U1 | A1 | B4 | | D27 | — | F1 | U4 |
| PN17 | U1 | A1 | E1 | B4 | D2 | H4 | F3 | U4 |
| PN18 | U1 | A1 | E1 | B4 | D2 | H1 | F1 | U4 |

| | | | | | | | |
|---|---|---|---|---|---|---|---|
| PN19 | U1 | A1 | B5 | D21 | H1 | F1 | U4 |
| PN20 | U1 | A1 | B6 | D21 | H1 | F1 | U4 |
| PN21 | U1 | A1 | B7 | D21 | H1 | F1 | U4 |
| PN22 | U1 | A2 | B8 | D1 | H3 | F2 | U4 |
| PN23 | U1 | A2 | B8 | — | H1 | F1 | U4 |
| PN24 | U1 | A1 | B4 | — | H5 | F4 | U4 |
| PN28 | U1 | A2 | B8 | — | H3 | F2 | U4 |
| PN29 | U1 | A2 | B8 | D1 | H1 | F1 | U4 |

TABLE C
Nucleotide Sequences of C-Fusion Constructs

| Ref No. | 5′UTR | Signal | N′-RBD | N′-Linker | Hinge | Fc | 3′UTR |
|---|---|---|---|---|---|---|---|
| PC8 | U1 | A1 | H1 | F1 | — | B4 | U4 |
| PC9 | U1 | A1 | H1 | F1 | D3 | B4 | U4 |
| PC10 | U1 | A1 | H1 | F1 | D18 | B4 | U4 |
| PC11 | U1 | A1 | H1 | F1 | D27 | B4 | U4 |

TABLE D
Nucleotide Sequences of Double-Fusion Constructs

| | | | N′- | N′- | | | C′- | C′- | |
|---|---|---|---|---|---|---|---|---|---|
| Ref No | 5′UTR | Signal | RBD | Linker | Hinge | Fc | Linker | RBD | 3′UTR |
| PS8 | — | A1 | B4 | D3 | H1 | F1 | D27 | B1 | — |
| PS9 | U1 | A1 | B1 | D3 | H1 | F1 | D27 | B4 | U4 |
| PS10 | — | A1 | B4 | D3 | — | F1 | D27 | B1 | — |
| PS11 | — | A1 | B1 | D3 | — | F1 | D27 | B4 | — |
| PS12 | U1 | A1 | B9 | D3 | H1 | F1 | D27 | B1 | U4 |
| PS13 | — | A1 | B9 | D28 | H1 | F1 | D27 | B1 | — |
| PS14 | U1 | A1 | B9 | D3 | H1 | F1 | D3 | B1 | U4 |
| PS15 | — | A1 | B9 | D27 | H1 | F1 | D4 | B1 | — |
| PS16 | U1 | A1 | B1 | D3 | H1 | F1 | D27 | B9 | U4 |
| PS18 | — | A4 | B26 | D30 | H8 | F9 | D31 | B2 | — |
| PS19 | — | A5 | B27 | D32 | H9 | F10 | D33 | B28 | — |
| PS20 | — | A6 | B29 | D34 | H10 | F11 | D35 | B3 | — |
| PS21 | U1 | A1 | B9 | D3 | H7 | F1 | D27 | B1 | U4 |
| PS22 | U1 | A1 | B9 | D3 | H1 | F5 | D27 | B1 | U4 |
| PS23 | U1 | A1 | B9 | D3 | H1 | F6 | D27 | B1 | U4 |
| PS24 | U1 | A1 | B9 | D3 | H1 | F7 | D27 | B1 | U4 |
| PS25 | U1 | A1 | B9 | D3 | H1 | F8 | D27 | B1 | U4 |
| PS26 | U1 | A1 | B10 | D29 | H1 | F1 | D3 | B1 | U4 |
| PS27 | U1 | A1 | B10 | D29 | H1 | F1 | D19 | B1 | U4 |
| PS28 | U1 | A1 | B9 | D3 | H1 | F1 | D27 | B1 | U5 |
| PS29 | U1 | A1 | B9 | D3 | H1 | F1 | D27 | B1 | U6 |
| PS30 | U2 | A1 | B9 | D3 | H1 | F1 | D27 | B1 | U4 |
| PS31 | U2 | A1 | B9 | D3 | H1 | F1 | D27 | B1 | U5 |
| PS32 | U2 | A1 | B9 | D3 | H1 | F1 | D27 | B1 | U6 |
| PS33 | U3 | A1 | B9 | D3 | H1 | F1 | D27 | B1 | U4 |
| PS34 | U1 | A1 | B9 | D20 | H1 | F1 | D3 | B4 | U4 |
| PS35 | U1 | A1 | B9 | D1 | H1 | F1 | D3 | B4 | U4 |
| PS36 | U1 | A1 | B10 | D20 | H1 | F1 | D3 | B4 | U4 |
| PS37 | U1 | A1 | B10 | D1 | H1 | F1 | D3 | B4 | U4 |
| PS38 | U1 | A1 | B11 | D20 | H1 | F1 | D3 | B4 | U4 |
| PS39 | U1 | A1 | B11 | D1 | H1 | F1 | D3 | B4 | U4 |
| PS40 | U1 | A1 | B11 | — | H1 | F1 | D3 | B4 | U4 |
| PS41 | U1 | A1 | B9 | D1 | H6 | F2 | D3 | B4 | U4 |
| PS42 | U1 | A1 | B10 | D20 | H6 | F2 | D3 | B4 | U4 |
| PS43 | U1 | A1 | B10 | D1 | H6 | F2 | D3 | B4 | U4 |
| PS44 | U1 | A1 | B11 | D1 | H6 | F2 | D3 | B4 | U4 |
| PS45 | U1 | A1 | B11 | D1 | H2 | F1 | D3 | B4 | U4 |
| PS46 | U1 | A1 | B12 | D5 | H1 | F1 | D3 | B4 | U4 |
| PS47 | U1 | A1 | B14 | D20 | H1 | F1 | D3 | B4 | U4 |
| PS48 | U1 | A1 | B14 | D1 | H1 | F1 | D3 | B4 | U4 |
| PS49 | U1 | A1 | B14 | — | H1 | F1 | D3 | B4 | U4 |
| PS50 | U1 | A1 | B12 | D1 | H6 | F2 | D3 | B4 | U4 |
| PS51 | U1 | A1 | B13 | D20 | H6 | F2 | D3 | B4 | U4 |
| PS52 | U1 | A1 | B13 | D1 | H6 | F2 | D3 | B4 | U4 |
| PS63 | U1 | A1 | B14 | D1 | H6 | F2 | D3 | B4 | U4 |
| PS53 | U1 | A1 | B14 | D1 | H2 | F1 | D3 | B4 | U4 |
| PS54 | U1 | A1 | B12 | — | H1 | F1 | D3 | B4 | U4 |
| PS55 | U1 | A1 | B12 | D20 | H1 | F1 | D3 | B4 | U4 |
| PS56 | U1 | A1 | B12 | D1 | H1 | F1 | D3 | B4 | U4 |
| PS57 | U1 | A1 | B13 | D20 | H1 | F1 | D3 | B4 | U4 |
| PS58 | U1 | A1 | B13 | D1 | H1 | F1 | D3 | B4 | U4 |
| PS59 | U1 | A1 | B9 | D3 | H1 | F5 | D3 | B1 | U4 |
| PS60 | U1 | A1 | B9 | D3 | H1 | F7 | D3 | B1 | U4 |
| PS61 | U1 | A1 | B12 | D5 | H1 | F5 | D3 | B4 | U4 |
| PS62 | U1 | A1 | B12 | D5 | H1 | F7 | D3 | B4 | U4 |
| PS64 | U1 | A1 | B12 | D5 | H1 | F5 | D3 | B4 | U6 |
| PS65 | U1 | A1 | B12 | D5 | H1 | F7 | D3 | B4 | U6 |
| PS66 | U1 | A1 | B12 | D20 | H1 | F5 | D3 | B4 | U4 |
| PS67 | U1 | A1 | B12 | D20 | H1 | F7 | D3 | B4 | U4 |
| PS68 | U1 | A1 | B12 | D20 | H1 | F5 | D3 | B4 | U6 |
| PS69 | U1 | A1 | B12 | D20 | H1 | F7 | D3 | B4 | U6 |
| PS70 | U1 | A1 | B12 | D20 | H1 | F5 | D27 | B4 | U4 |
| PS61- | U1 | A7 | B32 | D7 | H12 | F13 | D8 | B33 | U4 |
| opti-1 | | | | | | | | | |
| PS66- | U1 | A9 | B34 | D23 | H13 | F16 | D9 | B35 | U4 |
| opti-1 | | | | | | | | | |
| PS61- | U1 | A6 | B36 | D10 | H10 | F14 | D11 | B37 | U4 |
| opti-2 | | | | | | | | | |
| PS66- | U1 | A6 | B36 | D24 | H10 | F17 | D11 | B37 | U4 |
| opti-2 | | | | | | | | | |
| PS61- | U1 | A8 | B38 | D12 | H14 | F15 | D13 | B39 | U4 |
| opti-3 | | | | | | | | | |
| PS66- | U1 | A10 | B40 | D25 | H15 | F18 | D14 | B41 | U4 |
| opti-3 | | | | | | | | | |
| PS71 | U1 | A1 | B12 | D5 | H1 | F5 | D27 | B4 | U4 |
| PS71- | U1 | A7 | B32 | D7 | H12 | F22 | D38 | B33 | U4 |
| opti-1 | | | | | | | | | |
| PS71- | U1 | A6 | B36 | D10 | H10 | F14 | D27 | B37 | U4 |
| opti-2 | | | | | | | | | |
| PS72 | U1 | A1 | B15 | D5 | H1 | F5 | D27 | B4 | U4 |
| PS72- | U1 | A11 | B42 | D15 | H16 | F19 | D36 | B43 | U4 |
| opti-2 | | | | | | | | | |
| PS73 | U1 | A1 | B15 | D20 | H1 | F5 | D3 | B4 | U4 |
| PS73- | U1 | A12 | B30 | D22 | H11 | F12 | D6 | B31 | U4 |
| opti-1 | | | | | | | | | |
| PS73- | U1 | A13 | B44 | D26 | H17 | F20 | D16 | B45 | U4 |
| opti-2 | | | | | | | | | |
| PS74 | U1 | A1 | B9 | D3 | H1 | F5 | D27 | B1 | U4 |
| PS75 | U1 | A1 | B9 | D3 | H1 | F5 | D27 | B25 | U4 |
| PS76 | U1 | A12 | B31 | D22 | H11 | F12 | D6 | B30 | U4 |
| PS75- | U1 | A14 | B46 | D17 | H18 | F21 | D37 | B48 | U4 |
| opti-1 | | | | | | | | | |
| PS76- | U1 | A13 | B47 | D26 | H17 | F20 | D16 | B49 | U4 |
| opti-1 | | | | | | | | | |
| PS77 | U1 | A3 | B30 | D22 | H11 | F12 | D6 | B31 | U4 |
| PS78 | U1 | A1 | B9 | D3 | H1 | F5 | D27 | B16 | U4 |
| PS79 | U1 | A1 | B4 | D18 | H1 | F1 | D3 | B16 | U4 |
| PS80 | U1 | A1 | B4 | D18 | H1 | F1 | D27 | B16 | U4 |
| PS81 | U1 | A3 | B9 | D3 | H1 | F5 | D27 | B16 | U4 |
| PS82 | U1 | A3 | B9 | D3 | H1 | F5 | D27 | B25 | U4 |
| PS83 | U1 | A3 | B16 | D7 | H12 | F22 | D38 | B33 | U4 |
| PS84 | U1 | A1 | B9 | D3 | H1 | F5 | D27 | B17 | U4 |
| PS85 | U1 | A1 | B9 | D3 | H1 | F5 | D27 | B18 | U4 |
| PS86 | U1 | A1 | B9 | D3 | H1 | F5 | D27 | B19 | U4 |
| PS87 | U1 | A1 | B9 | D3 | H1 | F5 | D27 | B20 | U4 |
| PS88 | U1 | A1 | B9 | D3 | H1 | F5 | D27 | B21 | U4 |
| PS89 | U1 | A3 | B50 | D22 | H11 | F12 | D6 | B31 | U4 |
| PS95 | U1 | A1 | B9 | D3 | H1 | F5 | D27 | B24 | U4 |
| PS96 | U1 | A1 | B9 | D3 | H1 | F5 | D27 | B22 | U4 |
| PS97 | U1 | A1 | B9 | D3 | H1 | F5 | D27 | B23 | U4 |

The peptide constructs described in Tables E to H were prepared.

TABLE E
Amino Acid Control Constructs that Encode from 5′ to 3′:

| Ref No | Signal | RBD |
|---|---|---|
| AAPS1 | S1 | R4 |
| AAPS2 | S1 | R12 |
| AAPS3 | S4 | R1 |
| AAPS4 | S4 | R1 |

TABLE F
N-Fusion Constructs that Encode from 5′ to
3′: signal peptide-RBD-Linker-Hinge-Fc

| Ref No | Signal | N′-RBD | | N-Linker | Hinge | Fc |
|---|---|---|---|---|---|---|
| AAPN8 | S1 | R4 | | — | I1 | G1 |
| AAPN9 | S1 | R4 | | K3 | I1 | G1 |
| AAPN10 | S1 | R4 | | K4 | I1 | G1 |
| AAPN11 | S1 | R4 | | K5 | I1 | G1 |
| AAPN12 | S1 | M1 | R4 | K3 | I1 | G1 |
| AAPN13 | S1 | R4 | | — | — | G1 |
| AAPN14 | S1 | R4 | | K3 | — | G1 |
| AAPN15 | S1 | R4 | | K4 | — | G1 |
| AAPN16 | S1 | R4 | | K5 | — | G1 |
| AAPN17 | S1 | M1 | R4 | K2 | I4 | G3 |
| AAPN18 | S1 | M1 | R4 | K2 | I1 | G1 |

| | | | | | |
|---|---|---|---|---|---|
| AAPN19 | S1 | R5 | K4 | I1 | G1 |
| AAPN20 | S1 | R6 | K4 | I1 | G1 |
| AAPN21 | S1 | R7 | K4 | I1 | G1 |
| AAPN22 | S2 | R8 | K1 | I3 | G2 |
| AAPN23 | S2 | R8 | — | I1 | G1 |
| AAPN24 | S1 | R4 | — | I5 | G4 |
| AAPN28 | S2 | R8 | — | I3 | G2 |
| AAPN29 | S2 | R8 | K1 | I1 | G1 |

TABLE G
C′-Fusion Constructs that Encode from 5′
to 3′: signal peptide-Fc-Linker-RBD

| Ref No | Signal | Hinge | Fc | C′-Linker | C′-RBD |
|---|---|---|---|---|---|
| AAPC8 | S1 | I1 | G1 | — | R4 |
| AAPC9 | S1 | I1 | G1 | K3 | R4 |
| AAPC10 | S1 | I1 | G1 | K4 | R4 |
| AAPC11 | S1 | I1 | G1 | K5 | R4 |

TABLE H
Double-Fusion Constructs that Encode from 5′ to
3′: signal peptide-RBD-Linker-Fc-Linker-RBD

| | | N′-RBD | | | | | C′RBD |
|---|---|---|---|---|---|---|---|
| | | [Antigen | N′-Linker | | | C′-Linker | [Antigen |
| | Signal | Region | [Linker- | | | [Linker- | Region |
| Ref # | Peptide | (RBD)-1] | 1] | Hinge | Fc | 2] | (RBD)-2] |
| AAPS8 | S1 | R4 | K3 | I1 | G1 | K5 | R1 |
| AAPS9 | S1 | R1 | K3 | I1 | G1 | K5 | R4 |
| AAPS10 | S1 | R4 | K3 | — | G1 | K5 | R1 |
| AAPS11 | S1 | R1 | K3 | — | G1 | K5 | R4 |
| AAPS12 | S1 | R4 | K3 | I1 | G1 | K5 | R1 |
| AAPS13 | S1 | R4 | K5 | I1 | G1 | K5 | R1 |
| AAPS14 | S1 | R4 | K3 | I1 | G1 | K3 | R1 |
| AAPS15 | S1 | R4 | K5 | I1 | G1 | K3 | R1 |
| AAPS16 | S1 | R1 | K3 | I1 | G1 | K5 | R4 |
| AAPS18 | S1 | R4 | K5 | I1 | G1 | K5 | R2 |
| AAPS19 | S1 | R7 | K5 | I1 | G1 | K5 | R2 |
| AAPS20 | S1 | R7 | K5 | I1 | G1 | K5 | R3 |
| AAPS21 | S1 | R4 | K3 | I7 | G1 | K5 | R1 |
| AAPS22 | S1 | R4 | K3 | I1 | G5 | K5 | R1 |
| AAPS23 | S1 | R4 | K3 | I1 | G6 | K5 | R1 |
| AAPS24 | S1 | R4 | K3 | I1 | G7 | K5 | R1 |
| AAPS25 | S1 | R4 | K3 | I1 | G8 | K5 | R1 |
| AAPS26 | S1 | R7 | K5 | I1 | G1 | K3 | R1 |
| AAPS27 | S1 | R7 | K5 | I1 | G1 | K4 | R1 |
| AAPS28 | S1 | R4 | K3 | I1 | G1 | K5 | R1 |
| AAPS29 | S1 | R4 | K3 | I1 | G1 | K5 | R1 |
| AAPS30 | S1 | R4 | K3 | I1 | G1 | K5 | R1 |
| AAPS31 | S1 | R4 | K3 | I1 | G1 | K5 | R1 |
| AAPS32 | S1 | R4 | K3 | I1 | G1 | K5 | R1 |
| AAPS33 | S1 | R4 | K3 | I1 | G1 | K5 | R1 |
| AAPS34 | S1 | R4 | K4 | I1 | G1 | K3 | R4 |
| AAPS35 | S1 | R4 | K1 | I1 | G1 | K3 | R4 |
| AAPS36 | S1 | R7 | K4 | I1 | G1 | K3 | R4 |
| AAPS37 | S1 | R7 | K1 | I1 | G1 | K3 | R4 |
| AAPS38 | S1 | R8 | K4 | I1 | G1 | K3 | R4 |
| AAPS39 | S1 | R8 | K1 | I1 | G1 | K3 | R4 |
| AAPS40 | S1 | R8 | — | I1 | G1 | K3 | R4 |
| AAPS41 | S1 | R4 | K1 | I6 | G2 | K3 | R4 |
| AAPS42 | S1 | R7 | K4 | I6 | G2 | K3 | R4 |
| AAPS43 | S1 | R7 | K1 | I6 | G2 | K3 | R4 |
| AAPS44 | S1 | R8 | K1 | I6 | G2 | K3 | R4 |
| AAPS45 | S1 | R8 | K1 | I2 | G1 | K3 | R4 |
| AAPS46 | S1 | R9 | K3 | I1 | G1 | K3 | R4 |
| AAPS47 | S1 | R11 | K4 | I1 | G1 | K3 | R4 |
| AAPS48 | S1 | R11 | K1 | I1 | G1 | K3 | R4 |
| AAPS49 | S1 | R11 | — | I1 | G1 | K3 | R4 |
| AAPS50 | S1 | R9 | K1 | I6 | G2 | K3 | R4 |
| AAPS51 | S1 | R10 | K4 | I6 | G2 | K3 | R4 |
| AAPS52 | S1 | R10 | KI | I6 | G2 | K3 | R4 |
| AAPS63 | S1 | R11 | K1 | I6 | G2 | K3 | R4 |
| AAPS53 | S1 | R11 | K1 | I2 | G1 | K3 | R4 |
| AAPS54 | S1 | R9 | — | I1 | G1 | K3 | R4 |
| AAPS55 | S1 | R9 | K4 | I1 | G1 | K3 | R4 |
| AAPS56 | S1 | R9 | K1 | I1 | G1 | K3 | R4 |
| AAPS57 | S1 | R10 | K4 | I1 | G1 | K3 | R4 |
| AAPS58 | S1 | R10 | K1 | I1 | G1 | K3 | R4 |
| AAPS59 | S1 | R4 | K3 | I1 | G5 | K3 | R1 |
| AAPS60 | S1 | R4 | K3 | I1 | G7 | K3 | R1 |
| AAPS61 | S1 | R9 | K3 | I1 | G5 | K3 | R4 |
| AAPS62 | S1 | R9 | K3 | I1 | G7 | K3 | R4 |
| AAPS64 | S1 | R9 | K3 | I1 | G5 | K3 | R4 |
| AAPS65 | S1 | R9 | K3 | I1 | G7 | K3 | R4 |
| AAPS66 | S1 | R9 | K4 | I1 | G5 | K3 | R4 |
| AAPS67 | S1 | R9 | K4 | I1 | G7 | K3 | R4 |
| AAPS68 | S1 | R9 | K4 | I1 | G5 | K3 | R4 |
| AAPS69 | S1 | R9 | K4 | I1 | G7 | K3 | R4 |
| AAPS70 | S1 | R9 | K4 | I1 | G5 | K5 | R4 |
| AAPS61- | S1 | R9 | K3 | I1 | G5 | K3 | R4 |
| opti-1 | | | | | | | |
| AAPS66- | S1 | R9 | K4 | I1 | G5 | K3 | R4 |
| opti-1 | | | | | | | |
| AAPS61- | S1 | R9 | K3 | I1 | G5 | K3 | R4 |
| opti-2 | | | | | | | |
| AAPS66- | S1 | R9 | K4 | I1 | G5 | K3 | R4 |
| opti-2 | | | | | | | |
| AAPS61- | S1 | R9 | K3 | I1 | G5 | K3 | R4 |
| opti-3 | | | | | | | |
| AAPS66- | S1 | R9 | K4 | I1 | G5 | K3 | R4 |
| opti-3 | | | | | | | |
| AAPS71 | S1 | R9 | K3 | I1 | G5 | K5 | R4 |
| AAPS71- | S1 | R9 | K3 | I1 | G5 | K6 | R4 |
| opti-1 | | | | | | | |
| AAPS71- | S1 | R9 | K3 | I1 | G5 | K5 | R4 |
| opti-2 | | | | | | | |
| AAPS72 | S1 | R12 | K3 | I1 | G5 | K5 | R4 |
| AAPS72- | S1 | R12 | K3 | I1 | G5 | K5 | R4 |
| opti-2 | | | | | | | |
| AAPS73 | S1 | R12 | K4 | I1 | G5 | K3 | R4 |
| AAPS73- | S1 | R12 | K4 | I1 | G5 | K3 | R4 |
| opti-1 | | | | | | | |
| AAPS73- | S1 | R12 | K4 | I1 | G5 | K3 | R4 |
| opti-2 | | | | | | | |
| AAPS74 | S1 | R4 | K3 | I1 | G5 | K5 | R1 |
| AAPS75 | S1 | R4 | K3 | I1 | G5 | K5 | R12 |
| AAPS76 | S1 | R4 | K4 | I1 | G5 | K3 | R12 |
| AAPS75- | S1 | R4 | K3 | I1 | G5 | K5 | R12 |
| opti-1 | | | | | | | |
| AAPS76- | S1 | R4 | K4 | I1 | G5 | K3 | R12 |
| opti-1 | | | | | | | |
| AAPS77 | S3 | R12 | K4 | I1 | G5 | K3 | R4 |
| AAPS78 | S1 | R4 | K3 | I1 | G5 | K5 | R12 |
| AAPS79 | S1 | R4 | K4 | I1 | G1 | K3 | R12 |
| AAPS80 | S1 | R4 | K4 | I1 | G1 | K5 | R12 |
| AAPS81 | S3 | R4 | K3 | I1 | G5 | K5 | R12 |
| AAPS82 | S3 | R4 | K3 | I1 | G5 | K5 | R12 |
| AAPS83 | S3 | R12 | K3 | I1 | G5 | K6 | R4 |
| AAPS84 | S1 | R4 | K3 | I1 | G5 | K5 | R13 |
| AAPS85 | SI | R4 | K3 | I1 | G5 | K5 | R14 |
| AAPS86 | S1 | R4 | K3 | I1 | G5 | K5 | R15 |
| AAPS87 | S1 | R4 | K3 | I1 | G5 | K5 | R16 |
| AAPS88 | S1 | R4 | K3 | I1 | G5 | K5 | R17 |
| AAPS89 | S3 | R21 | K4 | I1 | G5 | K3 | R4 |
| AAPS95 | S1 | R4 | K3 | I1 | G5 | K5 | R20 |
| AAPS96 | SI | R4 | K3 | I1 | G5 | K5 | R18 |
| AAPS97 | S1 | R4 | K3 | I1 | G5 | K5 | R19 |

### Example 2: Assay of Polynucleotides

Polynucleotide constructs according to Example 1 were assayed in HEK293T cells to determine expression yields of the antigenic proteins of the disclosure.

Transfection: HEK293T cells were seeded in 12-well plates at 350,000 cells/well with 5% FBS and 1% antibiotics. Eighteen hours later, cells were transfected with Lipofectamine 3000 (Thermo Fisher Scientific) and individual plasmids. After 40 hours cultivation, the supernatant and cell lysate were collected and tested by ELISA or Western Blot as described below.

Western Blot: 36 μl cell lysate and 9 μl 5× loading buffer was mixed and boiled at 100° C. for 10 min. 45 μl mixture was loaded into the wells of the 10% SDS-PAGE gel, along with a molecular weight marker. The gel was run for 1-2 h at 100 V. PVDF was activated with methanol for 1 min and rinsed with transfer buffer before preparing the stack. Proteins were transferred to the membrane for 1 h at 100V.

The membrane was blocked for 1 h at room temperature or overnight at 4° C. using blocking buffer. Next, the membrane was incubated with 1:1000 dilutions of RBD monoclonal antibody (Sino Biological) in 3% BSA for 1 h at room temperature. The membrane was washed in three washes of TBST (5 min each). Then the membrane was incubated with 1:10000 dilutions of HRP-conjugated secondary antibody in 3% BSA for 1 h at room temperature and the membrane was washed in five washes of TBST (5 min each). Working Solution was prepared by mixing equal parts of the Stable Peroxide Solution and the Luminol/Enhancer Solution (Thermo SuperSignal™ West Pico PLUS Chemiluminescent Substrate). 0.1 mL Working Solution was used per cm² of membrane and was incubated for 5 minutes. The blot was removed from Working Solution and excess reagent was drained. The blot was placed in clear plastic wrap and exposed to imaging the system.

ELISA: First, the supernatant of transfected cell lysates were all collected. The Sino SARS-CoV-2 (2019-nCOV) Spike RBD ELISA Kit was used to detect the RBD antigen. Briefly, each well was washed three times with Wash Buffer, and 100 μL of each serially diluted protein standard or test sample was added per well, including a zero standard. The plate was covered/sealed and incubated for 2 hours at room temperature. The wash step was repeated and then 100 μL of Detection Antibody was added in working concentration to each well. The plate was covered/sealed and incubated for 1 hour at room temperature. The wash step was repeated and 200 μL of Substrate Solution was added to each well. After incubating for 20 minutes at room temperature (protected from light), 50 μL of Stop Solution was added to each well. If the color did not appear uniform, the plate was gently tapped to ensure thorough mixing. The optical density of each well was determined within 20 minutes, using a microplate reader set to 450 nm.

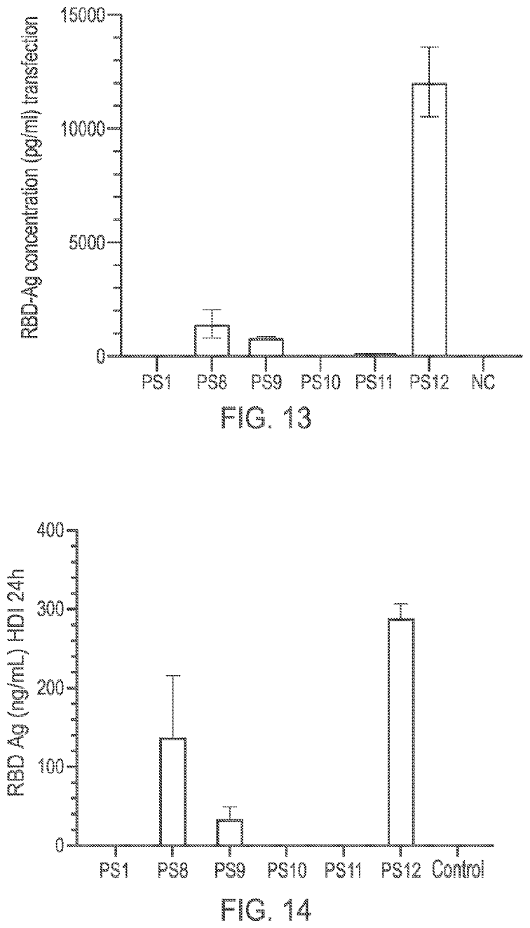

Tables 4, 5, and 6 and FIG. 13 show the results of these assays. Selected candidates provided expression yields in excess of 500 pg/ml. A construct containing a codon-optimized RBD (PS12) unexpectedly showed an advantageous combination of properties, including advantageous expression. For example, PS12 (SEQ ID NO: 34) showed surprising, unexpected, and advantageous properties, including over 15-fold higher expression level than an otherwise similar construct having a wild-type RBD sequence.

TABLE 4

| | | | | | | | L series Plasmid transfection | |
|---|---|---|---|---|---|---|---|---|
| | | | | | | | Cell | 293T |
| | | | | | | | 12 well | 35W/well |
| | | | | | | | (Sino SARS-CoV-2-Spike RBD ELISA kit | |
| | | | | | | | measure RBD Ag, Dilution: no dilution) | |
| | | | | | | | Sample | |
| | | | | | | | collection | |
| | | | | | | | time | 42 h |
| | | | | | Supernatant | | | |
| Conc | St. Curve | | | | OD | OD | | |
| (pg/ml) | OD Value | ID | Name | Dose | value-1 | value-2 | Conc. -1 | Conc. -2 |
| 500.0 | 1.950 | PN11 | sp-RBD-4GGGGS- | lug | 2.70 | 2.67 | 734.3 | 723.5 |
| | | | IgG4 | | | | | |
| 250.0 | 1.134 | PC11 | sp-IgG4-RBD- | lug | 1.92 | 2.24 | 508.5 | 600.2 |
| | | | 4GGGGS | | | | | |
| 125.0 | 0.632 | PN22 | HA sp-RBD(333- | lug | 2.02 | 2.22 | 538.6 | 594.9 |
| | | | 529)-GS-hinge-IgG1 | | | | | |
| 62.5 | 0.373 | PN23 | HA sp-RBD(333- | lug | 1.99 | 2.16 | 528.4 | 577.5 |
| | | | 529)-GS-hinge-IgG4 | | | | | |
| 31.3 | 0.257 | L-GFP | GFP | lug | 0.06 | 0.07 | ND | ND |
| 15.6 | 0.167 | R2 = 0.9908 | | | | | | |

ND = Not detectable

TABLE 5

| | | | | | | | L series Plasmid transfection | |
|---|---|---|---|---|---|---|---|---|
| | | | | | | | cell | 293T |
| | | | | | | | 12 well | 35W/well |
| | | | | | | | (Sino SARS-CoV-2-Spike RBD ELISA kit | |
| | | | | | | | measure RBD Ag, Dilution: 1:10 | |
| | | | | | | | Collection | |
| | | | | | | | time | 42 h |
| | | | | | Supernatant, | | Supernatant, measure | |
| | | | | | measure 1 | | 2 | |
| Conc | St. Curve | | | | OD | Converted | | Converted |
| (pg/ml) | OD Value | ID | Name | Dose | value | Conc. | OD value | Conc. |
| 500.0 | 2.334 | PN18 | stable-RBD-TEV- | lug | 0.27 | 420.3 | 0.24 | 364.4 |
| | | | hinge-Fc (IgG4) | | | | | |
| 250.0 | 1.186 | PN24 | RBD-hinge-Fc (IgG 1 | lug | 0.36 | 626.3 | 0.34 | 571.5 |
| | | | Acro) | | | | | |
| 125.0 | 0.669 | PS9 | RBDwt-2GGGGS- | lug | 0.46 | 848.9 | 0.43 | 785.7 |
| | | | hinge-Fc-4GGGGS- | | | | | |
| | | | RBD | | | | | |
| 62.5 | 0.359 | PS10 | RBD-2GGGGS- | lug | 0.07 | ND | 0.07 | ND |
| | | | nonhinge-Fc- | | | | | |
| | | | 4GGGGS-RBDwt | | | | | |
| 31.3 | 0.237 | PS12 | optiRBD-2GGGGS- | lug | 5.02 | 10968.2 | 6.00 | 13131.7 |
| | | | hinge-Fc-4GGGGS- | | | | | |
| | | | RBD wt | | | | | |
| 15.6 | 0.135 | L-GFP | GFP | lug | 0.06 | ND | 0.06 | ND |
| 7.8 | 0.099 | R2 = | | | | | | |
| | | 0.9995 | | | | | | |

ND = Not detectable

TABLE 6

| | | 293T cell culture supernatant | | | |
|---|---|---|---|---|---|
| | | | | Concentration-1 | Concentration-2 |
| Plasmid | Dose | OD-1 | OD-2 | (pg/ml) | (pg/ml) |
| PS1 | 1 ug | 0.06 | 0.05 | 13.2 | ND |
| PS8 | 1 ug | 0.54 | 0.91 | 979.8 | 1863.3 |
| PS9 | 1 ug | 0.46 | 0.43 | 848.9 | 785.7 |
| PS10 | 1 ug | 0.07 | 0.07 | ND | ND |
| PS11 | 1 ug | 0.06 | 0.08 | ND | 102.8 |
| PS12 | 1 ug | 5.02 | 6.00 | 10968.2 | 13131.7 |

Control construct PS1 contains a spike protein (Delta) and a single RBD(319-541, Delta) antigen region, but no linker, hinge, or Fc regions.
ND = Not detectable

### Example 3: In Vivo Antigen Expression

Polynucleotide constructs according to Tables A to D were assayed in BALB/C mice to determine in vivo antigen expression yields for the antigenic proteins of the disclosure.

Hydrodynamic injection (HDI): Plasmids for HDI assays were constructed by GenScript using a pCDNA 3.4 vector and the polynucleotide constructs according to Tables A to D. The total amount of a plasmid used for each mouse was 8% of the mouse's body weight, at a concentration of 5 μg/ml. Three mice were used for each group. Twenty-four hours after HDI, 20 μl of serum was collected from each mouse and the serum concentration of RBD-Fc fusion protein was detected by ELISA as described in Example 2 and the results are shown in Tables 7 and 8 and FIG. 14.

TABLE 7
HDI-L Series Plasmid In Vivo Antigen Expression
(Sino SARS-CoV-2-Spike RBD ELISA kit measure RBD Ag) Dilution: (1:200)
HDI 10 ug/ml (1 day post dosing/vaccination) BALB/C mice

| ID | | | | | | | | | | | |
|---|---|---|---|---|---|---|---|---|---|---|---|
| | St. Curve | | | | | | | | | | |
| Conc | OD | PN11 | PN18 | PN22 | PN23 | PN24 | PC11 | PS9 | PS10 | PS12 | Saline |
| (pg/ml) | Value | OD value | | | | | | | | | |
| 500.0 | 1.815 | 3.5146 | 0.1204 | 3.257 | 5.9365 | 0.2095 | 5.0454 | 0.002 | 5.2502 | 0.7805 | ND |
| 250.0 | 0.993 | 3.4051 | 0.0722 | 2.4537 | 5.9365 | 0.3925 | 3.5916 | 0.0008 | 5.9365 | 1.0477 | ND |
| 125.0 | 0.505 | 3.8376 | 0.0437 | 5.9365 | 5.1966 | 0.227 | 5.9365 | 0.001 | 4.7871 | 0.0661 | ND |
| 62.5 | 0.255 | Back Calculated Concentration (ng/ml) | | | | | | | | | |
| 31.3 | 0.126 | 189.95 | 5.84 | 175.98 | 321.33 | 10.67 | 272.99 | ND | 284.10 | 41.65 | ND |
| 15.6 | 0.063 | 184.01 | 3.22 | 132.41 | 321.33 | 20.60 | 194.13 | ND | 321.33 | 56.14 | ND |
| 7.8 | 0.004 | 207.48 | 1.68 | 321.33 | 281.19 | 11.62 | 321.33 | ND | 258.98 | 2.89 | ND |

| | | | | | | | | | | |
|---|---|---|---|---|---|---|---|---|---|---|
| geomean | 193.56 | 3.16 | 195.63 | 307.35 | 13.67 | 257.27 | ND | 287.01 | 18.91 | ND |
| mean | 193.81 | 3.58 | 209.91 | 307.95 | 14.30 | 262.82 | ND | 288.14 | 33.56 | ND |

ND = Not detectable

TABLE 8

| No. | PS1 | PS8 | PS9 | PS10 | PS11 | PS12 | saline |
|---|---|---|---|---|---|---|---|
| | OD value | | | | | | |
| Mouse 1 | ND | 0.133 | 0.781 | 0.002 | ND | 5.250 | ND |
| Mouse 2 | ND | 2.230 | 1.048 | 0.001 | ND | 5.937 | ND |
| Mouse 3 | ND | 1.136 | 0.066 | 0.001 | ND | 4.787 | ND |
| | concentration (ng/ml) | | | | | | |
| Mouse 1 | 0.656 | 2.026 | 41.646 | ND | ND | 284.100 | ND |
| Mouse 2 | ND | 275.224 | 56.140 | ND | ND | 321.328 | ND |
| Mouse 3 | ND | 132.632 | 2.894 | ND | ND | 258.980 | ND |

ND = Not detectable

### Example 4: Lipid Nanoparticle Formulation and Mouse Vaccination

Lipid-Nanoparticle (LNP)-mRNA: T7 DNA plasmids were synthesized and constructed by GenScript. Fragments containing the T7 promotor sequence were amplified by PCR using primer pairs F/R-RBD or F/R-Fc:

F:
TAATACGACTCACTATAG
R-RBD:
CTAGAAATTGACACATTTGTTTTTAACCAAATTAGTAG
R-Fc:
CTAGCCCAGAGACAGGCTCAGGGACT

The fragments were purified by Axygen Clean Up Kit and then added to the T7 IVT Kit reaction system (NEB). The UTP in the IVT system was replaced with pseudo-UTP and Trilink CleanCap was added at a final concentration of 4 mM. After 2 hours incubation at 37° C., 10 μl of 10× DNase I Buffer and 2 μl of RNase-free DNase I was added, mixed, and incubated at 37° C. for 15 minutes. Monarch® RNA Cleanup Kit (NEB) was used to purify the in vitro synthesized mRNA. Incubation of 5 units of the Poly(A) Polymerase (NEB) with 1-10 μg RNA in a 20 μl reaction at 37° C. for 30 minutes (with 1× Reaction buffer and 1 mM ATP) will result in a tail length of greater than 100 bases. The Monarch® RNA Cleanup Kit (NEB) was then used to purify the tailed mRNA.

Lipid-nanoparticle (LNP) formulations were prepared using a modified procedure of a method previously described for siRNA (Ickenstein and Garidel, Expert Opin Drug Deliv (2019) 16(11): 1205-1226). Briefly, lipids were dissolved in ethanol containing an ionizable lipid, 1,2-distearoyl-sn-glycero-3-phosphocholine (DSPC), cholesterol and PEG-lipid (with molar ratios of 50:10:38.5:1.5). The lipid mixture was combined with 20 mM citrate buffer (pH 4.0) containing mRNA at a ratio of 1:2 through a T-mixer. Formulations were then diafiltrated against 10× volume of PBS (pH 7.4) through 100 kD molecular weight cut-offs (Millipore) and concentrated to desired concentrations and stored at 4° C. until use. All formulations were tested for particle size, distribution, RNA concentration and encapsulation.

Mouse Vaccination: Female Balb/c mice (6-8 week-old) were purchased and were randomly divided into two groups (n=3/group). mRNA-LNP or LNP was intramuscularly administrated into animals (15 μg/mouse). The orbital blood was collected at 6 h/30 h after administration, centrifuged at 5,000 g at 4° C. for 10 minutes. Sera were collected and stored at −80° C. for further testing. RBD expression level was determined by ELISA as described in Example 2.

### Example 5: Expression of RBD-Fc Fusion Proteins of Varying Lengths

In vitro and in vivo expression levels of RBD-Fc fusion proteins with different lengths of RBD were assessed. The constructs contained different lengths of the SARS-CoV-2 S protein RBD. Specifically, the constructs contained SARS-CoV-2 S protein amino acids Gly311 to Asn532 (RBD 311-532), SARS-CoV-2 S protein amino acids Thr167 to Phe541 (RBD 167-541), SARS-CoV-2 S protein amino acids Arg319 to Lys537 (RBD 319-537), and SARS-CoV-2 S protein amino acids Arg319 to Phe541 (RBD 319-541).

The in vitro protein expression levels of RBD-Fc fusion proteins with different lengths of RBD were different.

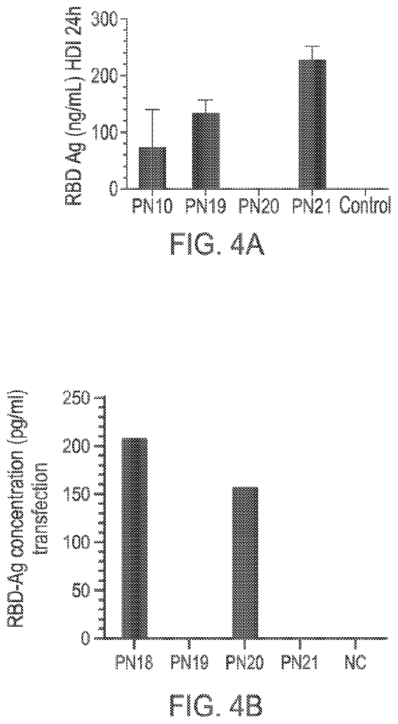

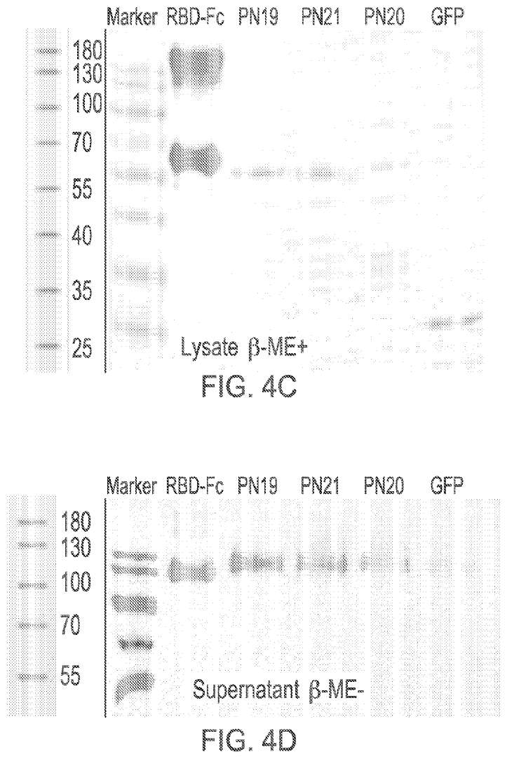

In vivo protein expression levels of RBD-Fc fusion proteins with different lengths of RBD were also different. As shown in FIG. 4A and FIG. 4B, high in vivo protein expression was demonstrated by constructs containing SARS-CoV-2 S protein amino acids Gly311 to Asn532 (RBD 311-532) and SARS-CoV-2 S protein amino acids Arg319 to Lys537 (RBD 319-537). A lower level of in vivo protein expression was demonstrated by a construct containing SARS-CoV-2 S protein amino acids Arg319 to Phe541 (RBD 319-541). In vivo protein expression for a construct containing SARS-CoV-2 S protein amino acids Thr167 to Phe541 (RBD 167-541) was similar to saline and GFP controls.

### Example 6: Expression of RBD-Fc and Fc-RBD Fusion Proteins Over Time

In vitro and in vivo expression levels of RBD-Fc and Fc-RBD fusion proteins were assessed.

High in vitro protein expression was demonstrated for constructs containing SARS-CoV-2 S protein RBD-Fc and Fc-RBD fusion proteins.

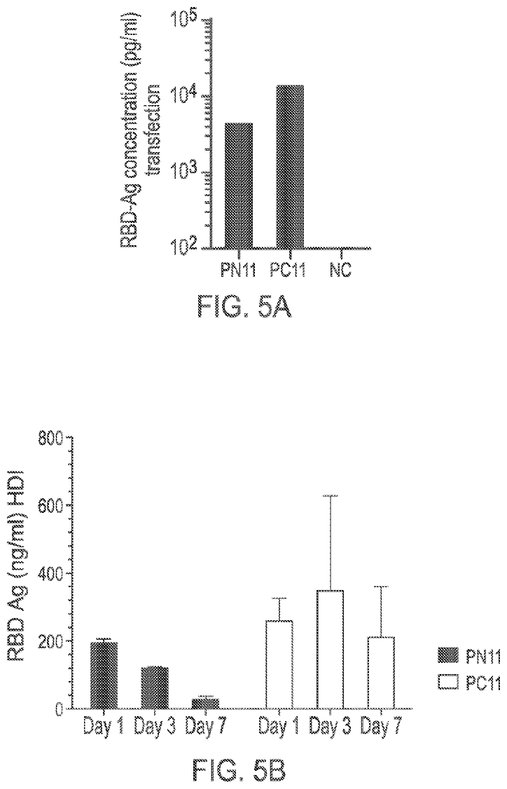

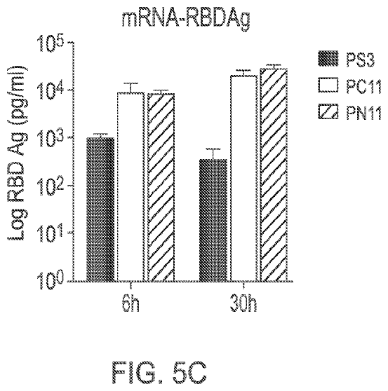

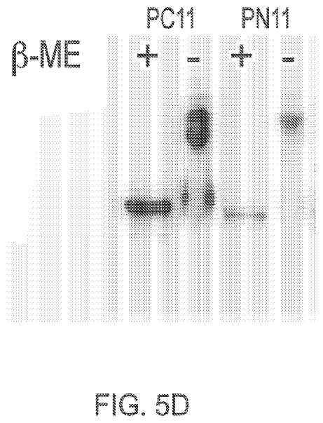

As shown in FIG. 5A, FIG. 5B, and FIG. 5C, high in vivo protein expression was demonstrated by constructs containing SARS-CoV-2 S protein RBD-Fc and Fc-RBD fusion proteins, with the Fc-RBD fusion protein expression being higher and being maintained for a longer time period.

### Example 7: Expression of Double RBD-Fc Fusion Proteins

In vitro and in vivo expression levels of double RBD-Fc fusion proteins were assessed.

High in vitro protein expression was demonstrated for constructs containing two SARS-CoV-2 S protein RBDs connected by an Fc domain.

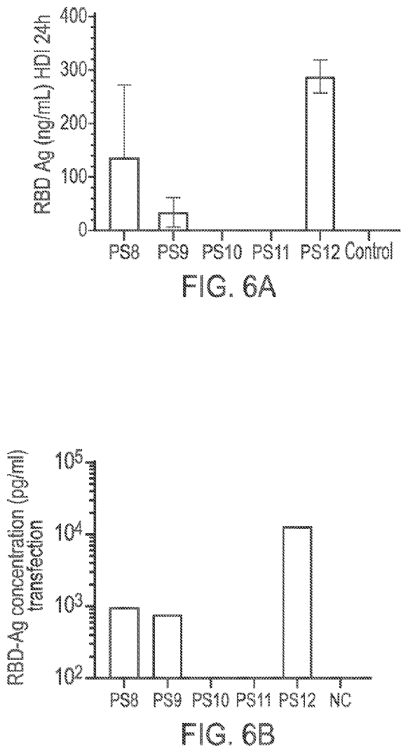

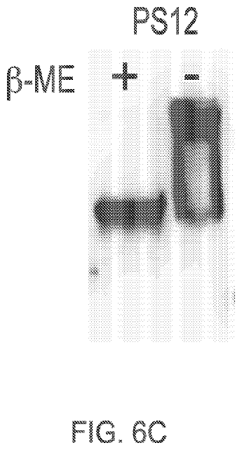

In vivo protein expression levels of constructs containing two different SARS-CoV-2 S protein RBDs connected by an Fc domain were assessed. Specifically, the constructs contained wild type SARS-CoV-2 S protein RBD (RBD_{WT}) and SARS-CoV-2 S protein RBD delta variant (RBD_{DELTA}). As shown in FIG. 6A and FIG. 6B, higher in vivo protein expression was demonstrated by constructs containing a RBD_{DELTA}-Fc-RBD_{WT} fusion protein compared to a RBD_{WT}-Fc-RBD_{DELTA} fusion protein.

### Example 8: Expression of RBD-Fc and Fc-RBD Fusion Proteins with Different Linker Lengths

In vitro and in vivo expression levels of RBD-Fc and Fc-RBD fusion proteins with different linker lengths were assessed.

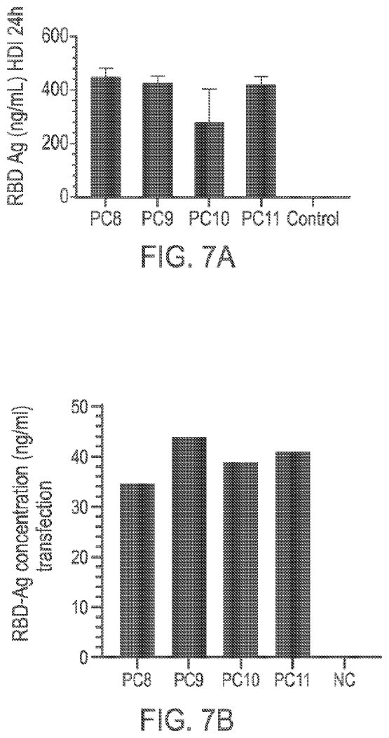

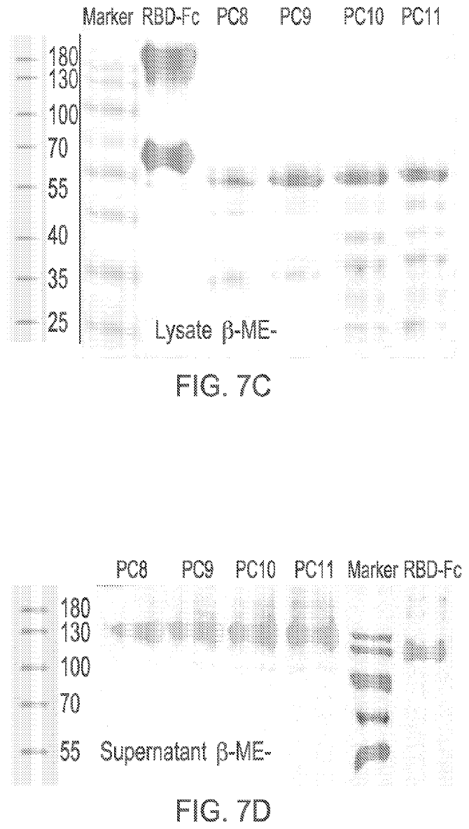

Linker length did not affect the expression level of Fc-RBD fusion proteins in vivo. Further, as shown in FIG. 7A and FIG. 7B, linker length did not affect the expression level of Fc-RBD fusion proteins in vitro.

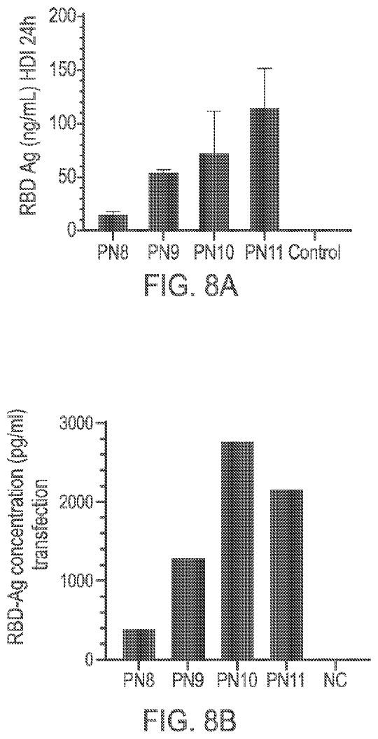

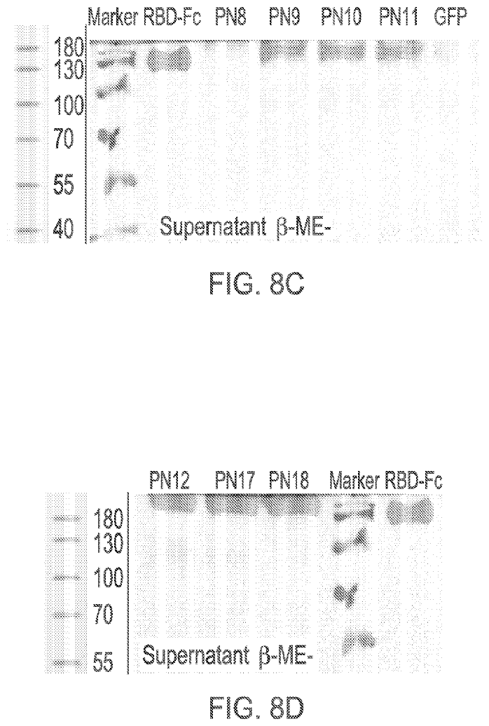

Linker length did affect the expression level of RBD-Fc fusion proteins in vivo, with longer linkers demonstrating higher in vivo expression levels. Further, as shown in FIG. 8A and FIG. 8B, linker length did affect the expression level of RBD-Fc fusion proteins in vitro, with longer linkers (e.g., constructs PN10 and PN11) demonstrating higher in vitro expression levels.

### Example 9: Effect of Hinge on Stability of Double RBD-Fc and RBD-Fc Fusion Proteins

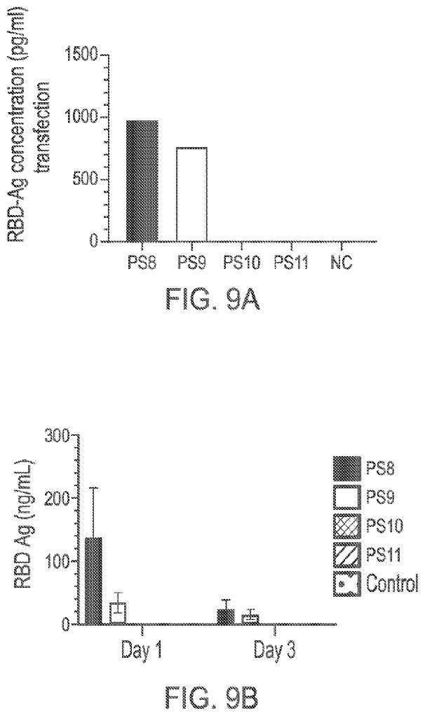

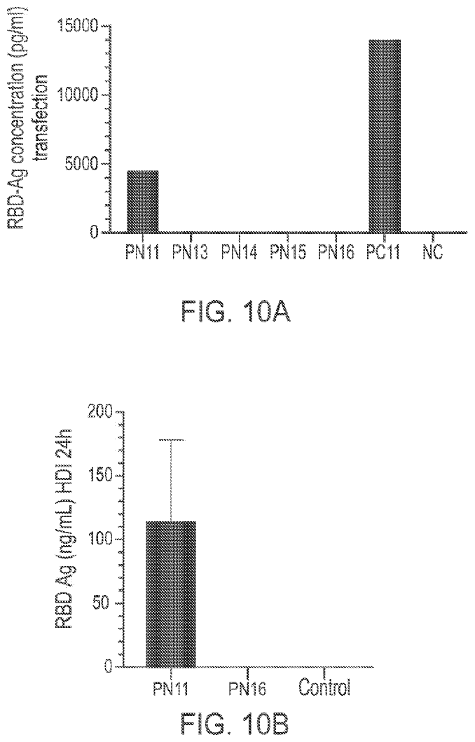

As shown in FIG. 9A and FIG. 9B, the hinge affects the stability of double-end RBD fused Fc proteins. Further, as shown in FIG. 10A and FIG. 10B, the hinge affects the stability of RBD-Fc fusion proteins.

### Example 10: Expression of RBD-Fc Fusion Proteins with IgG1 and IgG4 Fc

In vitro and in vivo expression levels of RBD-Fc fusion proteins with IgG1 and IgG4 Fc were assessed.

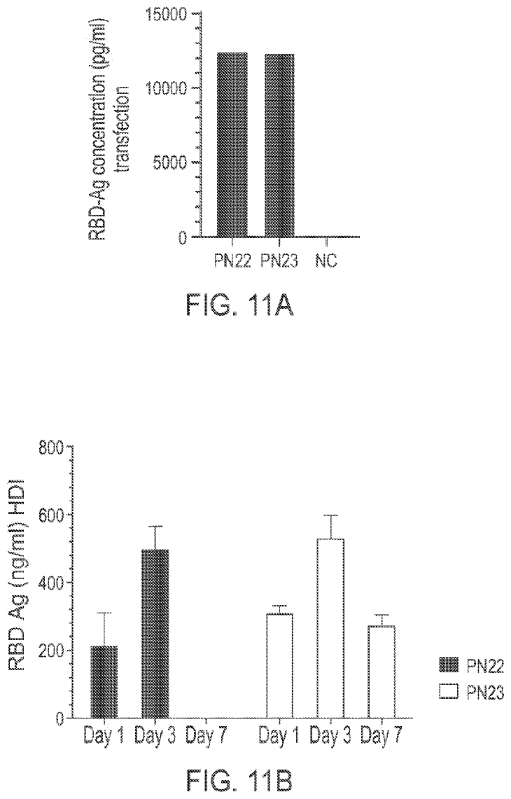

As shown in FIG. 11A, IgG4-Fc (F234A/L235A double mutant) fused antigens have similar in vitro expression levels compared to IgG1-Fc fused proteins. However, as shown in FIG. 11B, IgG4-Fc (F234A/L235A double mutant) fused antigens have significantly higher in vivo expression levels than IgG1-Fc fused proteins, and longer in vivo half-life.

### Example 11: Selection of SARS-CoV-2 RBD

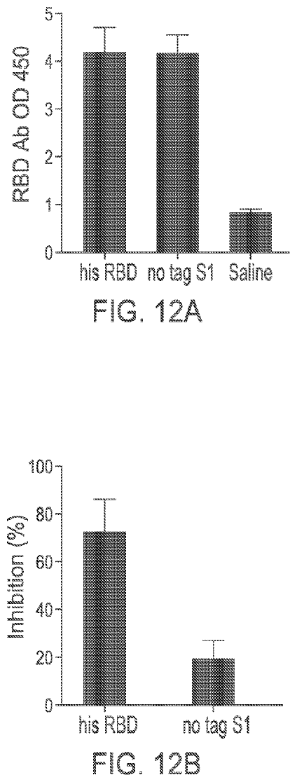

As shown in FIG. 12A, compared with the normal saline group, both his-RBD and no tag S1 protein induced higher levels of RBD antibodies (P<0.01), but FIG. 12B shows that his-RBD protein induced a significantly higher level of neutralizing antibody than that of no tag S1 protein (P<0.01).

RBD regions of different lengths were then selected for further development, including amino acids Thr167-Phe541, Gly311-Asn532, Arg319-Lys537, Arg319-Phe541, and Thr333-Lys529. Expression of the RBD antigen was assessed by in vitro transfection of 293T cells with each construct. The Arg319-Phe541, and Thr333-Lys529 RBDs both expressed RBD antigen at a level significantly higher than that of the GFP control group (P<0.01).

### Example 12: Antibody Expression of Double RBD-Fc Fusion Proteins

Lipid nanoparticle-mRNA (LNP-mRNA) in vivo expression level, anti-RBD antibody levels, and pseudovirus-based SARS-CoV-2 50% neutralizing antibody levels (pVNT₅₀) elicited by the LNP-mRNA in female Balb/c mice were evaluated.

5 μg LNP-mRNA was immunized to the mice by intramuscular injection (IM) on Day 0 and 14, respectively. LNP-mRNA (PS4) and phosphate buffer saline (PBS) with LNP components were set as positive and negative control group individually. The serum antigen expression level was measured at 6 h, 27 h, 3 d, 7 d, 21 d and serum anti-Delta RBD antibody levels were evaluated at 7 d, 14 d, 21 d, 28 d, 35 d post vaccination by enzyme-linked immune-sorbent assays (ELISA). Pseudovirus-based Delta and Omicron SARS-CoV-2 50% neutralization (pVNT₅₀) were measured using serum samples drawn 21 d, 28 d and 35 d post immunization.

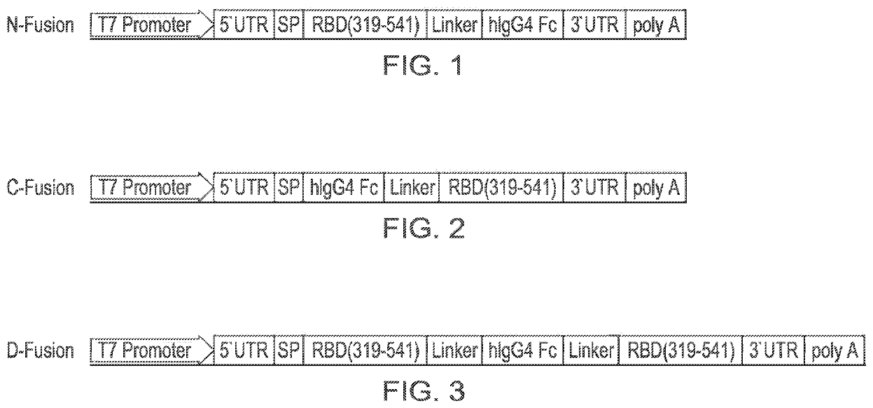

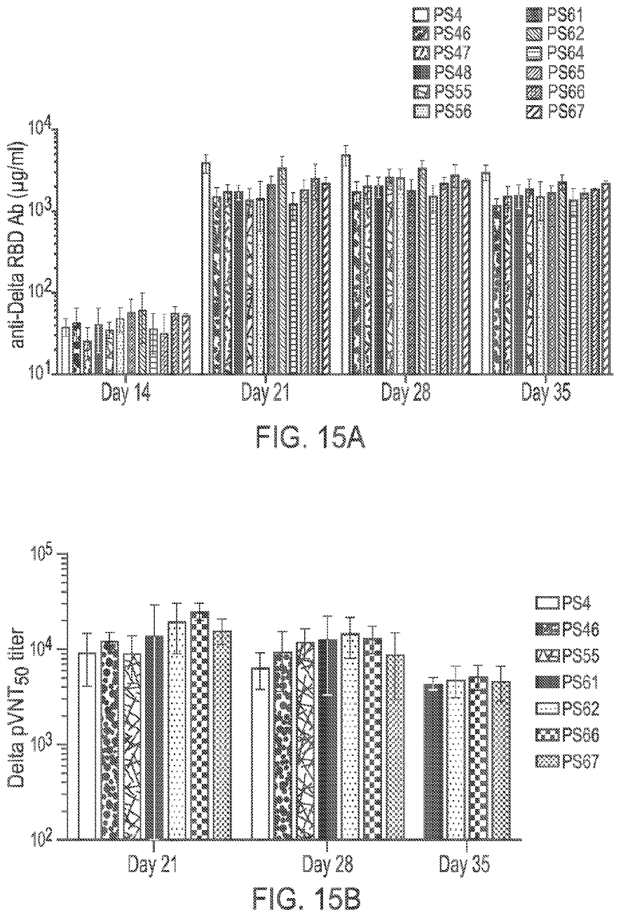

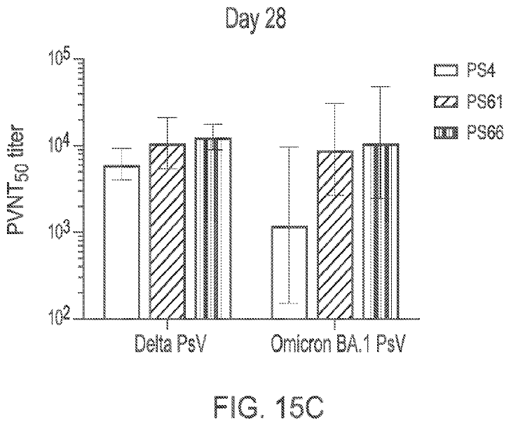

Results are shown for the following mRNA constructs: PS46, PS47, PS48, PS55, PS56, PS61, PS62, PS64, PS65, PS66, and PS67. The serum SARS-CoV-2 anti-Delta RBD antibody levels are shown in FIG. 15A. The IgG antibody levels generated by administration of these mRNA constructs after 28 days are 1.5×10³ to 3.4×10³ μg/mL, which are much higher than the 1×10² μg/mL peak levels previously seen mounted by humans after SARS-CoV-2 infections. [FIG. 1 of Iyer et al, Sci. Immunol. 10.1126/sciimmunol.abe0367 (2020)]. The Delta and Omicron BA.1, respectively, pseudovirus-based SARS-CoV-2 50% neutralization (pVNT₅₀) titer is shown in FIGS. 15B and 15C. The pseudovirus-based SARS-CoV-2 50% neutralization (pVNT₅₀) titer levels generated by administration of these mRNA constructs are 4.7×10³ to 2.5×10⁴ (Delta) and 1.4×10⁴ to 1.7×10⁴ (BA.1), which are much higher than the 5×10² to 2×10³ peak titer levels previously seen mounted by humans after SARS-CoV-2 infections [FIG. 3 of Iyer et al, Sci. Immunol. 10.1126/sciimmunol.abe0367 (2020)]. These mRNA constructs are therefore promising SARS-CoV-2 vaccine candidates.

### Example 13: Antibody Expression of Double RBD-Fc Fusion Proteins

Lipid nanoparticle-mRNA (LNP-mRNA) in vivo expression level, anti-RBD antibody levels, and pseudovirus-based SARS-CoV-2 50% neutralizing antibody levels (pVNT₅₀) elicited by the LNP-mRNA in female Balb/c mice were evaluated.

3 μg LNP-mRNA was immunized to the mice by intramuscular injection (IM) on Day 0 and 14, respectively. LNP-mRNA (PS1) and phosphate buffer saline (PBS) group were set as positive and negative control group individually. The serum antigen expression level was measured at 6 h, 30 h, 3 d, 7 d and the serum antibody were evaluated at 14 d, 21 d, 28 d, 35 d, 42 d post vaccination by enzyme-linked immune-sorbent assays (ELISA). Pseudovirus-based Delta SARS-CoV-2 50% neutralization (pVNT₅₀) was measured using serum samples drawn 21 d post immunization.

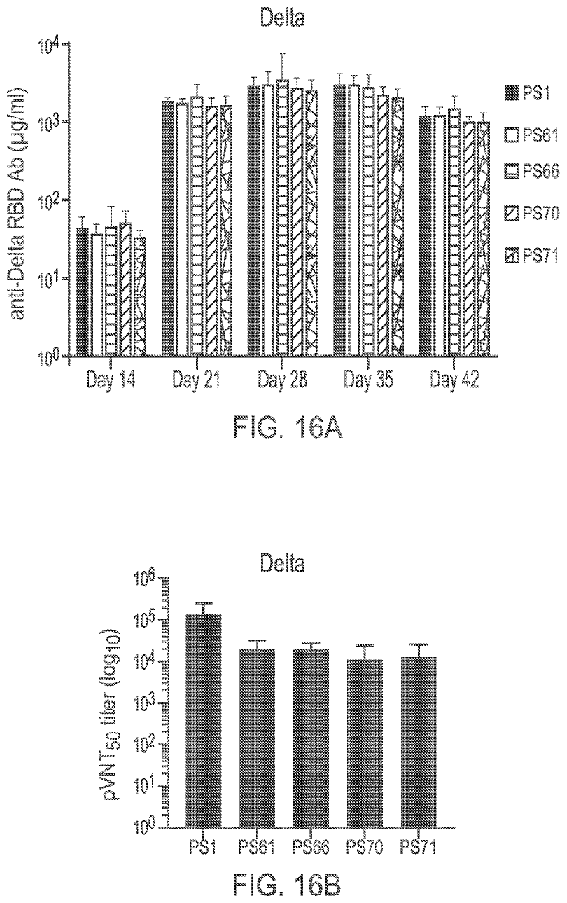

Results are shown for the following mRNA constructs: PS61, PS66, PS70, and PS71. The serum SARS-CoV-2 anti-Delta RBD antibody levels are shown in FIG. 16A. The IgG antibody levels generated by administration of these mRNA constructs after 28 days are 2.6×10³ to 3.5×10³ μg/mL, which are much higher than the 1×10² μg/mL peak levels previously seen mounted by humans after SARS-CoV-2 infections. [FIG. 1 of Iyer et al, Sci. Immunol. 10.1126/sciimmunol.abe0367 (2020)]. The Delta pseudovirus-based SARS-CoV-2 50% neutralization (pVNT₅₀) titer is shown in FIG. 16B. The pseudovirus-based SARS-CoV-2 50% neutralization (pVNT₅₀) titer levels generated by administration of these mRNA constructs are 1.3×10⁴ to 2.9×10⁴, which are much higher than the 5×10² to 2×10³ peak titer levels previously seen mounted by humans after SARS-CoV-2 infections [FIG. 3 of Iyer et al, Sci. Immunol. 10.1126/sciimmunol.abe0367 (2020)]. These mRNA constructs are therefore promising SARS-CoV-2 vaccine candidates.

### Example 14: Antibody Expression of Double RBD-Fc Fusion Proteins

Lipid nanoparticle-mRNA (LNP-mRNA) in vivo expression level, anti-RBD antibody levels, and pseudovirus-based SARS-CoV-2 50% neutralizing antibody levels (pVNT₅₀) elicited by the LNP-mRNA in female Balb/c mice were evaluated.

3 μg LNP-mRNA was immunized to the mice by intramuscular injection (IM) on Day 0 and Day 14, respectively. LNP-mRNA (PS2/PS12) and phosphate buffer saline (PBS) group were set as positive and negative control group individually. The serum antigen expression level was measured at 6 h, 30 h, 3 d and the serum antibody were evaluated at 14 d, 21 d, 28 d, 35 d post vaccination by enzyme-linked immune-sorbent assays (ELISA). Pseudovirus-based Delta and Omicron SARS-CoV-2 50% neutralization (pVNT₅₀) was measured using serum samples drawn 28 d and 35 d post immunization.

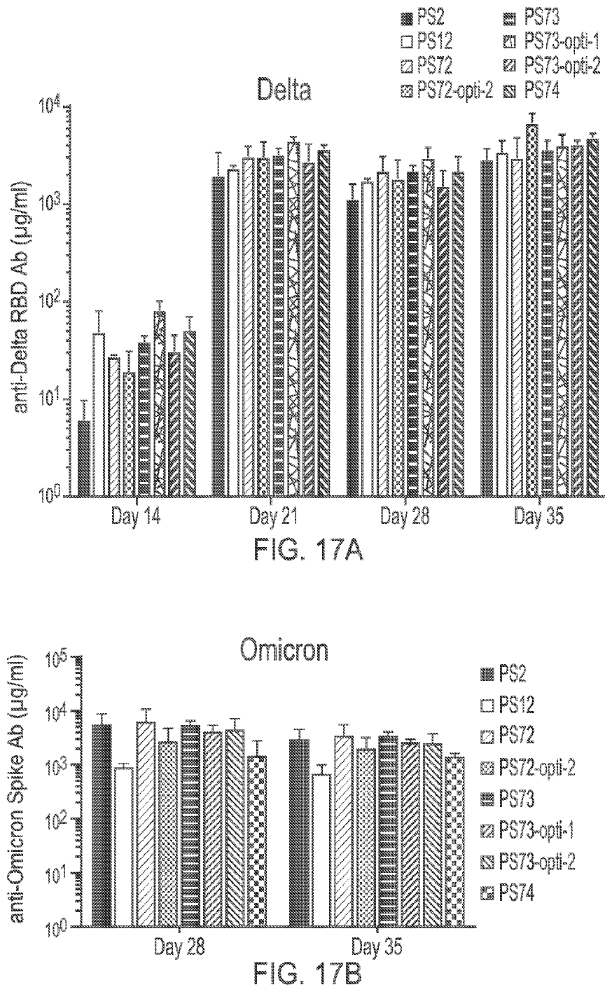

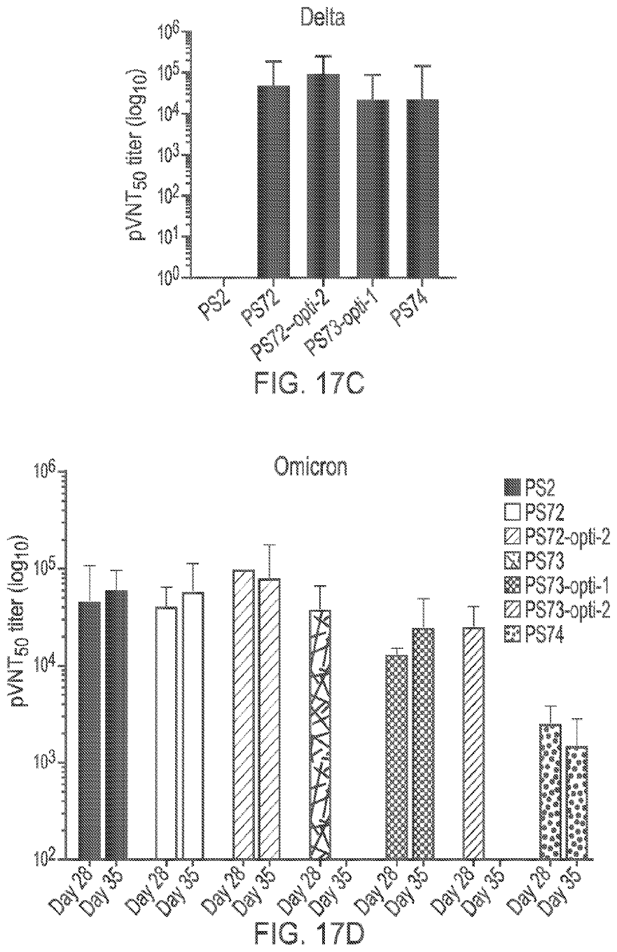

Results are shown for the following mRNA constructs: PS72, PS72-opti-2, PS73, PS73-opti-1, PS73-opti-2, and PS74. The serum SARS-CoV-2 anti-Delta RBD and anti-Omicron BA.1 RBD antibody levels are shown in FIGS. 17A and 17B. The IgG antibody levels generated by administration of these mRNA constructs after 28 days are 1.5×10³ to 3×10³ (Delta) and 2.2×10³ to 6.4×10³ (BA.1) μg/mL, which are much higher than the 1×10² μg/mL peak levels previously seen mounted by humans after SARS-CoV-2 infections. [FIG. 1 of Iyer et al, Sci. Immunol. 10.1126/sciimmunol.abe0367 (2020)]. Delta and Omicron BA.1, respectively, pseudovirus-based SARS-CoV-2 50% neutralization (pVNT₅₀) titer levels are shown in FIGS. 17C and 17D. The pseudovirus-based SARS-CoV-2 50% neutralization (pVNT₅₀) titer levels generated by administration of these mRNA constructs are 5.7×10³ to 2.4×10⁵ (Delta) and 2.2×10² to 1.9×10⁵ (BA.1), which are much higher than the 5×10² to 2×10³ peak titer levels previously seen mounted by humans after SARS-CoV-2 infections [FIG. 3 of Iyer et al, Sci. Immunol. 10.1126/sciimmunol.abe0367 (2020)]. These mRNA constructs are therefore promising SARS-CoV-2 vaccine candidates.

### Example 15: Antibody Expression of Double RBD-Fc Fusion Proteins

Lipid nanoparticle-mRNA (LNP-mRNA) in vivo expression level, anti-RBD antibody levels, and pseudovirus-based SARS-CoV-2 50% neutralizing antibody levels (pVNT₅₀) elicited by the LNP-mRNA in female Balb/c mice were evaluated.

3 μg LNP-mRNA was immunized to the mice by intramuscular injection (IM) on Day 0 and 14, respectively. LNP-mRNA (PS4/PS1/PS2/PS12) and phosphate buffer saline (PBS) group were set as positive and negative control group individually. The serum antigen expression level was measured at 6 h, 30 h and the serum antibody (Delta RBD/WT Spike/Omicron Spike) was evaluated at 14 d, 21 d, 28 d, 35 d post vaccination by enzyme-linked immune-sorbent assays (ELISA). Pseudovirus-based Delta and Omicron SARS-CoV-2 50% neutralization (pVNT₅₀) was measured using serum samples drawn 35 d post immunization.

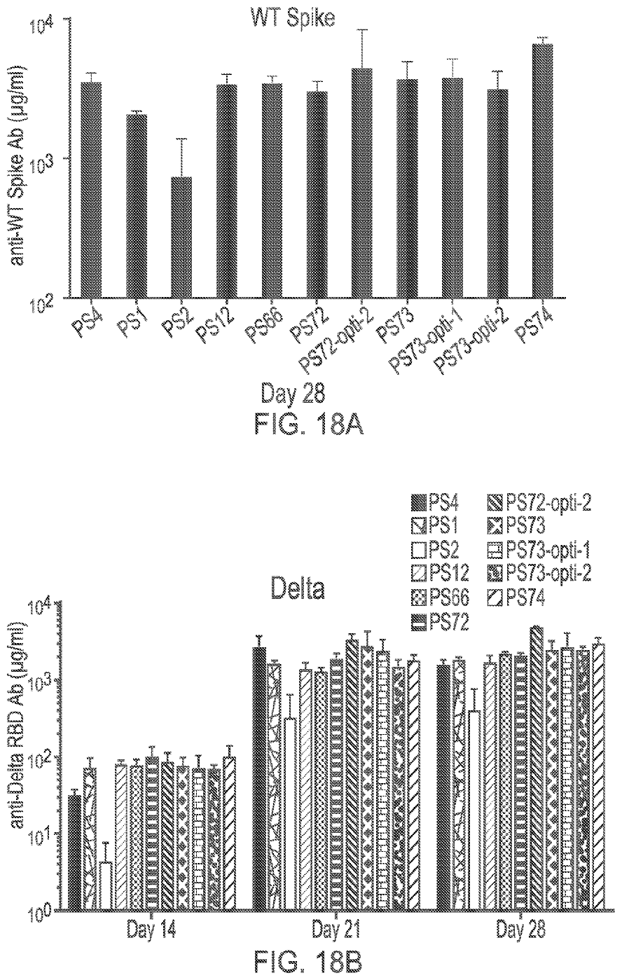

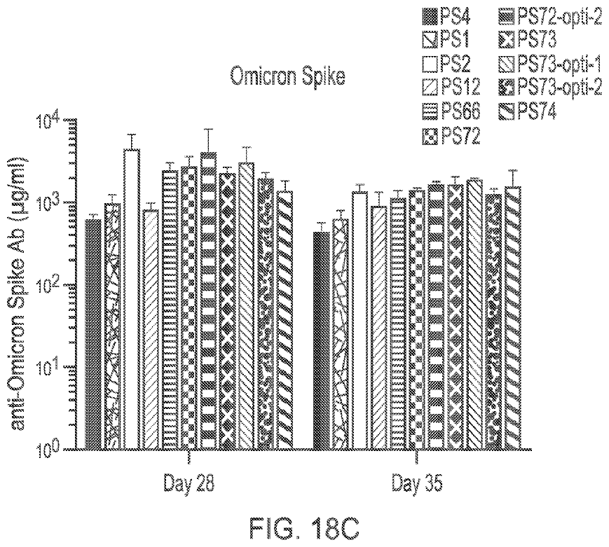

Results are shown for the following mRNA constructs: PS66, PS72, PS72-opti-2, PS73, PS73-opti-1, PS73-opti-2, and PS74. The serum SARS-CoV-2 anti-Wild-Type RBD, SARS-CoV-2 anti-Delta RBD, and anti-Omicron BA.1 RBD antibody levels are described in FIGS. 18A, 18B, and 18C. The IgG antibody levels generated by administration of these mRNA constructs after 28 days are 3.1×10³ to 6.7×10³ (WT), 2.1×10³ to 4.9×10³ (Delta) and 1.4×10³ to 4.2×10³ (BA.1) μg/mL, which are much higher than the 1×10² μg/mL peak levels previously seen mounted by humans after SARS-CoV-2 infections. [FIG. 1 of Iyer et al, Sci. Immunol. 10.1126/sciimmunol.abe0367 (2020)]. These mRNA constructs are therefore promising SARS-CoV-2 vaccine candidates.

### Example 16: Antibody Expression of Double RBD-Fc Fusion Proteins

Lipid nanoparticle-mRNA (LNP-mRNA) in vivo expression level, anti-RBD antibody levels, and pseudovirus-based SARS-CoV-2 50% neutralizing antibody levels (pVNT₅₀) elicited by the LNP-mRNA in female Balb/c mice were evaluated.

5 μg LNP-mRNA was immunized to the mice by intramuscular injection (IM) on Day 0 and Day 14, respectively. LNP-mRNA (PS1, PS2) and phosphate buffer saline (PBS) group were set as positive and negative control group individually. The serum RBD-specific antibody was evaluated on 7 d, 14 d, 21 d, 27 d and 35 d post vaccination by enzyme-linked immune-sorbent assays (ELISA). Pseudovirus-based SARS-CoV-2 50% neutralization (pVNT₅₀) was measured using serum samples drawn 14 d, 21 d, 27 d and 35 d post immunization.

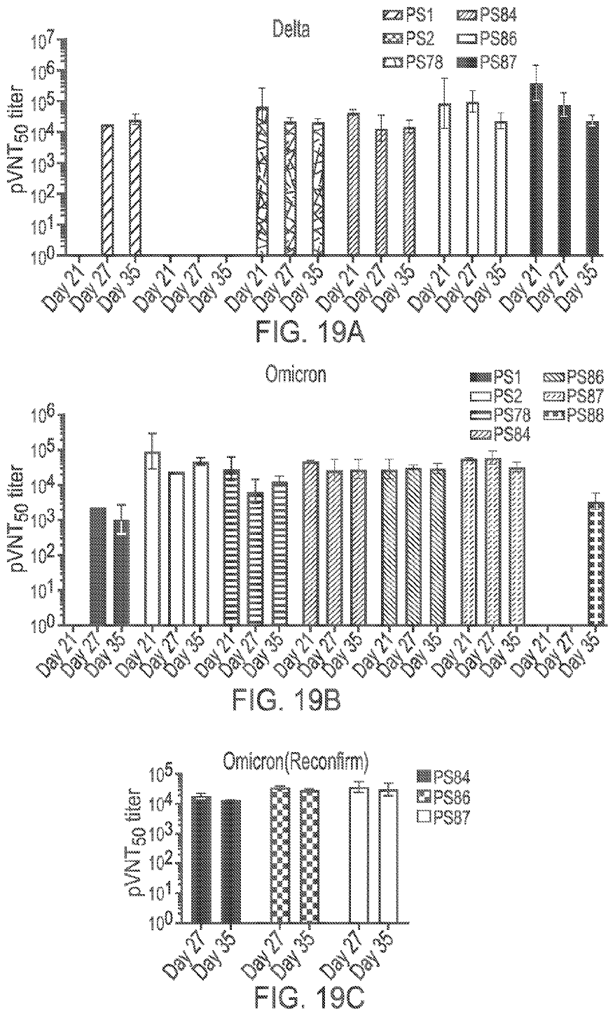

Results are shown for the following mRNA constructs: PS78, PS84, PS86, PS87, and PS88. FIGS. 19A, 19B, and 19C describe Delta and Omicron BA.1, respectively, pseudovirus-based SARS-CoV-2 50% neutralization (pVNT₅₀) titer generated in mice. The pseudovirus-based SARS-CoV-2 50% neutralization (pVNT₅₀) titer levels generated by administration of these mRNA constructs are 1.7×10³ to 4.7×10⁵ (Delta) and 1.9×10³ to 5.7×10⁵ (BA.1), which are much higher than the 5×10² to 2×10³ peak titer levels previously seen mounted by humans after SARS-CoV-2 infections [FIG. 3 of Iyer et al, Sci. Immunol. 10.1126/sciimmunol.abe0367 (2020)]. These mRNA constructs are therefore promising SARS-CoV-2 vaccine candidates.

### Example 17: Antibody Expression of Double RBD-Fc Fusion Proteins

Lipid nanoparticle-mRNA (LNP-mRNA) in vivo expression level, anti-RBD antibody levels, and pseudovirus-based SARS-CoV-2 50% neutralizing antibody levels (pVNT₅₀) elicited by the LNP-mRNA in female Balb/c mice were evaluated.

3 μg LNP-mRNA was immunized to the mice by intramuscular injection (IM) on day 0 and day 14, respectively. LNP-mRNA (PS1, PS2) and phosphate buffer saline (PBS) group were set as positive and negative control group individually. The serum antigen expression level was measured at 6 h and the serum antibody was evaluated at day 14, day 21, day 27 and day 35 post vaccination by enzyme-linked immune-sorbent assays (ELISA). Pseudovirus-based SARS-CoV-2 50% neutralization (pVNT₅₀) was measured using serum samples drawn day 21, day 27 and day 35 post immunization.

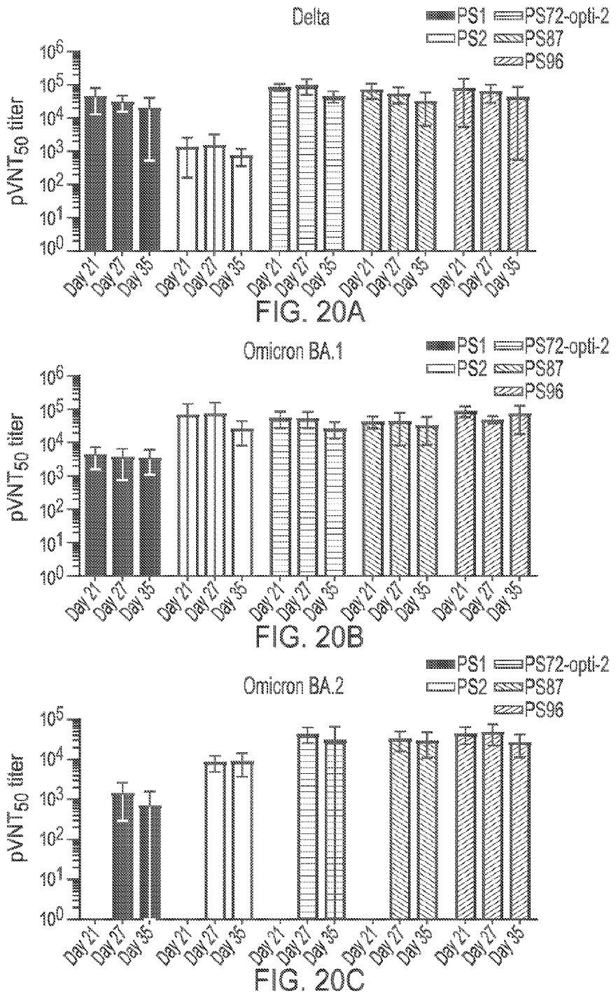

Results are shown for the following mRNA constructs: PS72-opti-2, PS87, and PS96. FIGS. 20A, 20B, and 20C describe Delta, Omicron BA.1, and Omicron BA.2, respectively, pseudovirus-based SARS-CoV-2 50% neutralization (pVNT₅₀) titer generated in mice. The pseudovirus-based SARS-CoV-2 50% neutralization (pVNT₅₀) titer levels generated by administration of these mRNA constructs are 2.6×10⁴ to 8.7×10⁴ (Delta), 2.5×10⁴ to 1.1×10⁵ (BA.1) and 2.3×10⁴ to 4.9×10⁴ (BA.2), which are much higher than the 5×10² to 2×10³ peak titer levels previously seen mounted by humans after SARS-CoV-2 infections [FIG. 3 of Iyer et al, Sci. Immunol. 10.1126/sciimmunol.abe0367 (2020)]. These mRNA constructs are therefore promising SARS-CoV-2 vaccine candidates.

### Example 18: Antibody Expression of Double RBD-Fc Fusion Proteins

Lipid nanoparticle-mRNA (LNP-mRNA) in vivo expression level, anti-RBD antibody levels, and pseudovirus-based SARS-CoV-2 50% neutralizing antibody levels (pVNT₅₀) elicited by the LNP-mRNA in female Balb/c mice were evaluated.

3 μg LNP-mRNA was immunized to the mice by intramuscular injection (IM) on day 0 and day 14, respectively. LNP-mRNA (PS1, PS2) and lipid component group were set as positive and negative control groups individually. The serum antigen expression levels were measured at 6 h, 30 h and day 3. Serum antibody levels were evaluated at day 14, day 21 and day 28 post vaccination by enzyme-linked immune-sorbent assays (ELISA). Pseudovirus-based SARS-CoV-2 50% neutralization (pVNT₅₀) was measured using serum samples drawn day 21 and day 28 post immunization.

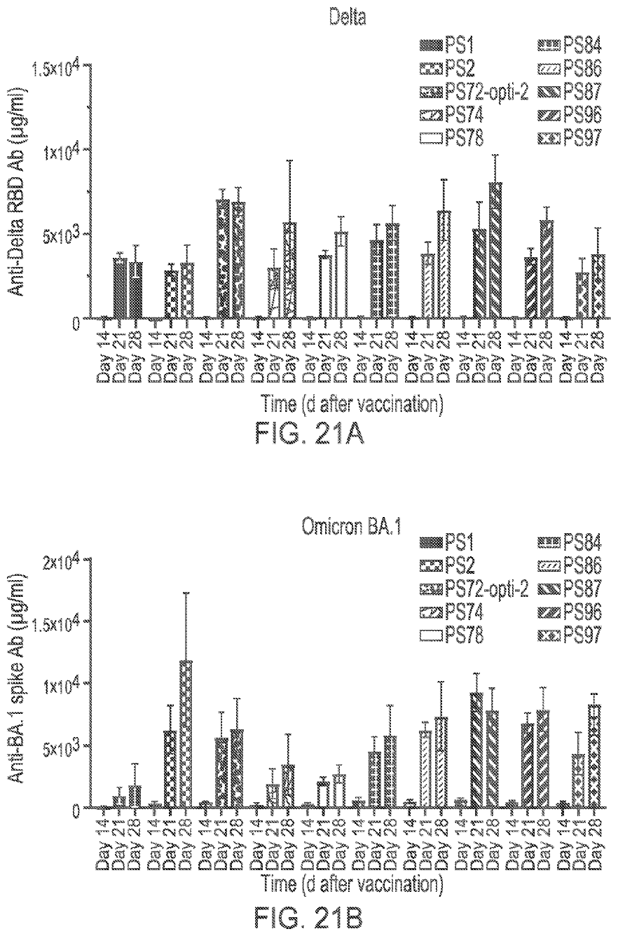

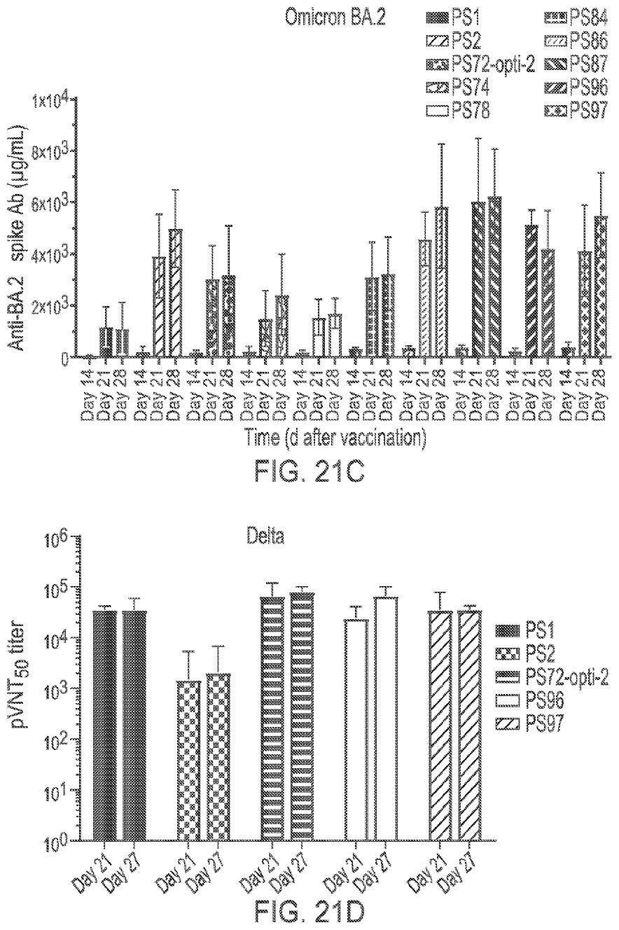

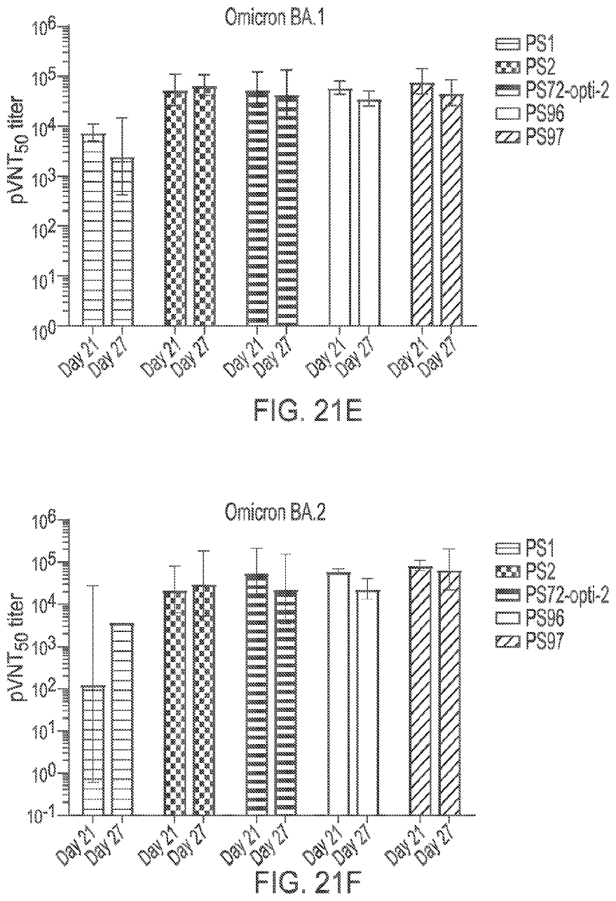

Results are shown for the following mRNA constructs: PS72-opti-2, PS74, PS78, PS84, PS86, PS87, PS96, and PS97. The serum SARS-CoV-2 anti-Delta RBD, anti-Omicron BA.1 RBD, and anti-Omicron BA.2 RBD antibody levels are shown in FIGS. 21A, 21B, and 21C. The IgG antibody levels generated by administration of these mRNA constructs after 28 days are 3.6×10³ to 8×10³ (Delta), 2.9×10³ to 8.2×10³ (BA.1) and 1.6×10³ to 6.1×10³ (BA.2) μg/mL, which are much higher than the 1×10² μg/mL peak levels previously seen mounted by humans after SARS-CoV-2 infections. [FIG. 1 of Iyer et al, Sci. Immunol. 10.1126/sciimmunol.abe0367 (2020)]. FIGS. 21D, 21E, and 21F describe Delta pseudovirus-based SARS-CoV-2 50% neutralization (pVNT₅₀) titer generated in mice. The pseudovirus-based SARS-CoV-2 50% neutralization (pVNT₅₀) titer levels generated by administration of these mRNA constructs are 2.5×10⁴ to 8.3×10⁴, which are much higher than the 5×10² to 2×10³ peak titer levels previously seen mounted by humans after SARS-CoV-2 infections [FIG. 3 of Iyer et al, Sci. Immunol. 10.1126/sciimmunol.abe0367 (2020)]. These mRNA constructs are therefore promising SARS-CoV-2 vaccine candidates.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. In addition, it is not necessary for a method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

### REFERENCES

Zhang Y, Zhou Z, Zhu S L, Zu X, Wang Z, Zhang L K, Wang W, Xiao G. A novel RSV F-Fc fusion protein vaccine reduces lung injury induced by respiratory syncytial virus infection. Antiviral Res. 2019 May; 165:11-22. doi: 10.1016/j.antiviral.2019.02.017. Epub 2019 Feb. 26. PMID: 30822450.
Diamos A G, Pardhe M D, Sun H, Hunter J G L, Kilbourne J, Chen Q, Mason H S. A Highly Expressing, Soluble, and Stable Plant-Made IgG Fusion Vaccine Strategy Enhances Antigen Immunogenicity in Mice Without Adjuvant. Front Immunol. 2020 Dec. 4; 11:576012. doi: 10.3389/fimmu.2020.576012. PMID: 33343565; PMCID: PMC7746858.
Li Y, Li R, Wang M, Liu Y, Yin Y, Zai X, Song X, Chen Y, Xu J, Chen W. Fc-Based Recombinant Henipavirus Vaccines Elicit Broad Neutralizing Antibody Responses in Mice. Viruses. 2020 Apr. 23; 12(4):480. doi: 10.3390/v12040480. PMID: 32340278; PMCID: PMC7232446.
Kim M Y, Copland A, Nayak K, Chandele A, Ahmed M S, Zhang Q, Diogo G R, Paul M J, Hofmann S, Yang M S, Jang Y S, Ma J K, Reljic R. Plant-expressed Fc-fusion protein tetravalent dengue vaccine with inherent adjuvant properties. Plant Biotechnol J. 2018 July; 16(7):1283-1294. doi: 10.1111/pbi.12869. Epub 2018 Feb. 1. PMID: 29223138; PMCID: PMC5999314.
Li J, Li X, Ma H, Ren X, Hao G, Zhang H, Zhao Z, Fang K, Li X, Rong Z, Sun S, Chen H, Qian P. Efficient mucosal vaccination of a novel classical swine fever virus E2-Fc fusion protein mediated by neonatal Fc receptor. Vaccine. 2020 Jun. 15; 38(29):4574-4583. doi: 10.1016/j.vaccine.2020.05.013. Epub 2020 May 7. PMID: 32417139.
Logan M, Law J, Wong J A J, Hockman D, Landi A, Chen C, Crawford K, Kundu J, Baldwin L, Johnson J, Dahiya A, LaChance G, Marcotrigiano J, Law M, Foung S, Tyrrell L, Houghton M. Native Folding of a Recombinant gpE1/gpE2 Heterodimer Vaccine Antigen from a Precursor Protein Fused with Fc IgG. J Virol. 2016 Dec. 16; 91(1):e01552-16. doi: 10.1128/JVI.01552-16. PMID: 27795422; PMCID: PMC5165201.
Shubin Z, Li W, Poonia B, Ferrari G, LaBranche C, Montefiori D, Zhu X, Pauza C D. An HIV Envelope gp120-Fc Fusion Protein Elicits Effector Antibody Responses in Rhesus Macaques. Clin Vaccine Immunol. 2017 Jun. 5; 24(6):e00028-17. doi: 10.1128/CVI.00028-17. PMID: 28404572; PMCID: PMC5461376.
Loureiro S, Ren J, Phapugrangkul P, Colaco C A, Bailey C R, Shelton H, Molesti E, Temperton N J, Barclay W S, Jones I M. Adjuvant-free immunization with hemagglutinin-Fc fusion proteins as an approach to influenza vaccines. J Virol. 2011 March; 85(6):3010-4. doi: 10.1128/JVI.01241-10. Epub 2010 Dec. 29. PMID: 21191017; PMCID: PMC3067967.
Ren W, Sun H, Gao G F, Chen J, Sun S, Zhao R, Gao G, Hu Y, Zhao G, Chen Y, Jin X, Fang F, Chen J, Wang Q, Gong S, Gao W, Sun Y, Su J, He A, Cheng X, Li M, Xia C, Li M, Sun L. Recombinant SARS-CoV-2 spike S1-Fc fusion protein induced high levels of neutralizing responses in nonhuman primates. Vaccine. 2020 Jul. 31; 38(35):5653-5658. doi: 10.1016/j.vaccine.2020.06.066. Epub 2020 Jun. 24. PMID: 32651113; PMCID: PMC7311893.
He Y, Zhou Y, Liu S, Kou Z, Li W, Farzan M, Jiang S. Receptor-binding domain of SARS-CoV spike protein induces highly potent neutralizing antibodies: implication for developing subunit vaccine. Biochem Biophys Res Commun. 2004 Nov. 12; 324(2):773-81. doi: 10.1016/j.bbrc.2004.09.106. PMID: 15474494; PMCID: PMC7092904.
Wang H, Yu J, Li L. A DNA vaccine encoding mutated HPV58 mE6E7-Fc-GPI fusion antigen and GM-CSF and B7.1. Onco Targets Ther. 2015 Oct. 23; 8:3067-77. doi: 10.2147/OTT.S84888. PMID: 26604780; PMCID: PMC4631422.
Zhao B, Zhang X, Krummenacher C, Song S, Gao L, Zhang H, Xu M, Feng L, Feng Q, Zeng M, Xu Y, Zeng Y. Immunization With Fc-Based Recombinant Epstein-Barr Virus gp350 Elicits Potent Neutralizing Humoral Immune Response in a BALB/c Mice Model. Front Immunol. 2018 May 1; 9:932. doi: 10.3389/fimmu.2018.00932. PMID: 29765376; PMCID: PMC5938345.
Gil M, Bieniasz M, Wierzbicki A, Bambach B J, Rokita H, Kozbor D. Targeting a mimotope vaccine to activating Fcgamma receptors empowers dendritic cells to prime specific CD8+ T cell responses in tumor-bearing mice. J Immunol. 2009 Nov. 15; 183(10):6808-18. doi: 10.4049/jimmunol.0900364. Epub 2009 Oct. 21. PMID: 19846865; PMCID: PMC2805007.
Zhang B, Kracker S, Yasuda T, Casola S, Vanneman M, Hömig-Hölzel C, Wang Z, Derudder E, Li S, Chakraborty T, Cotter S E, Koyama S, Currie T, Freeman G J, Kutok J L, Rodig S J, Dranoff G, Rajewsky K. Immune surveillance and therapy of lymphomas driven by Epstein-Barr virus protein LMP1 in a mouse model. Cell. 2012 Feb. 17; 148(4):739-51. doi: 10.1016/j.cell.2011.12.031. PMID: 22341446; PMCID: PMC3313622.
Morse M A, Bradley D A, Keler T, Laliberte R J, Green J A, Davis T A, Inman B A. CDX-1307: a novel vaccine under study as treatment for muscle-invasive bladder cancer. Expert Rev Vaccines. 2011 June; 10(6):733-42. doi: 10.1586/erv.11.20. PMID: 21692696.
Lu Z, Lee K J, Shao Y, Lee J H, So Y, Choo Y K, Oh D B, Hwang K A, Oh S H, Han Y S, Ko K. Expression of GA733-Fc fusion protein as a vaccine candidate for colorectal cancer in transgenic plants. J Biomed Biotechnol. 2012; 2012:364240. doi: 10.1155/2012/364240. Epub 2012 May 23. PMID: 22675251; PMCID: PMC3366255.
Nagarajan D, Pearson J, Brentville V, Metheringham R, Pockley A G, Durrant L, McArdle S E. ImmunoBody®-HAGE derived vaccine induces immunity to HAGE and delays the growth and metastasis of HAGE-expressing tumours in vivo. Immunol Cell Biol. 2021 Jun. 9. doi: 10.1111/imcb.12485. Epub ahead of print. PMID: 34105800.
Farsiani H, Mosavat A, Soleimanpour S, Sadeghian H, Akbari Eydgahi M R, Ghazvini K, Sankian M, Aryan E, Jamehdar S A, Rezaee S A. Fc-based delivery system enhances immunogenicity of a tuberculosis subunit vaccine candidate consisting of the ESAT-6:CFP-10 complex. Mol Biosyst. 2016 Jun. 21; 12(7):2189-201. doi: 10.1039/c6mb00174b. PMID: 27138226.
Zuo L, Cullen C M, DeLay M L, Thornton S, Myers L K, Rosloniec E F, Boivin G P, Hirsch R. A single-chain class II MHC-IgG3 fusion protein inhibits autoimmune arthritis by induction of antigen-specific hyporesponsiveness. J Immunol. 2002 Mar. 1; 168(5):2554-9. doi: 10.4049/jimmunol. 168.5.2554. PMID: 11859151.
Mi W, Wanjie S, Lo S T, Gan Z, Pickl-Herk B, Ober R J, Ward E S. Targeting the neonatal fc receptor for antigen delivery using engineered fc fragments. J Immunol. 2008 Dec. 1; 181(11):7550-61. doi: 10.4049/jimmunol.181.11.7550. PMID: 19017944; PMCID: PMC2738423.
Zhu D, Kepley C L, Zhang K, Terada T, Yamada T, Saxon A. A chimeric human-cat fusion protein blocks cat-induced allergy. Nat Med. 2005 April; 11(4):446-9. doi: 10.1038/nm1219. Epub 2005 Mar. 27. PMID: 15793580.
Eggel A, Buschor P, Baumann M J, Amstutz P, Stadler B M, Vogel M. Inhibition of ongoing allergic reactions using a novel anti-IgE DARPin-Fc fusion protein. Allergy. 2011 July; 66(7):961-8. doi: 10.1111/j.1398-9995.2011.02546.x. Epub 2011 Jan. 28. PMID: 21272035.

1. A messenger ribonucleic acid (mRNA) comprising: an open reading frame encoding a monomeric polypeptide chain comprising a first coronavirus spike protein receptor binding domain (RBD) area, a first linker, a hinge, a Fc domain, a second linker, and a second coronavirus spike protein receptor binding domain (RBD) area.

2. The mRNA of claim 1 comprising: an open reading frame encoding a monomeric polypeptide chain comprising a first coronavirus spike protein RBD, a first linker, a hinge, a Fc domain, a second linker, and a second coronavirus spike protein RBD.

3. The mRNA of claim 1, wherein the mRNA further comprises a 5′ untranslated region (5′-UTR).

4. (canceled)

5. The mRNA of claim 1, wherein the mRNA further comprises a 3′ untranslated region (3′-UTR).

6. (canceled)

7. The mRNA of claim 1, wherein the mRNA further comprises a poly(A) tail.

8. (canceled)

9. The mRNA of claim 1, wherein the mRNA further comprises a 5′ cap or 5′ cap analog.

10. (canceled)

11. The mRNA of claim 1, wherein the mRNA comprises a chemical modification.

12. The mRNA of claim 1, wherein the mRNA further comprises a ribonucleotide sequence encoding a signal peptide.

13. (canceled)

14. The mRNA of claim 12, wherein the signal peptide is a coronavirus spike protein signal peptide.

15-46. (canceled)

47. The mRNA of claim 1, wherein the first coronavirus spike protein receptor binding domain is a SARS-CoV-2 spike protein receptor binding domain.

48-49. (canceled)

50. The mRNA of claim 1, wherein the second coronavirus spike protein receptor binding domain is a SARS-CoV-2 spike protein receptor binding domain.

51-65. (canceled)

66. The mRNA of claim 1, wherein the mRNA encodes from 5′ to 3′: the first coronavirus spike protein receptor binding domain, the first linker, the hinge, the Fc domain, the second linker, and the second coronavirus spike protein receptor binding domain.

67-80. (canceled)

81. A monomeric polypeptide chain comprising a first coronavirus spike protein receptor binding domain area, a first linker, a hinge, a Fc domain, a second linker, and a second coronavirus spike protein receptor binding domain area.

82. The monomeric polypeptide chain of claim 81, comprising, from N-terminus to C-terminus, the first coronavirus spike protein receptor binding domain, the first linker, the hinge, the Fc domain, the second linker, and the second coronavirus spike protein receptor binding domain.

83-90. (canceled)

91. The monomeric polypeptide chain of claim 81, wherein the first coronavirus spike protein receptor binding domain is a SARS-CoV-2 spike protein receptor binding domain.

92-93. (canceled)

94. The monomeric polypeptide chain of claim 81, wherein the second coronavirus spike protein receptor binding domain is a SARS-CoV-2 spike protein receptor binding domain.

95-104. (canceled)

105. An antigenic protein comprising two monomeric polypeptide chains, wherein each monomeric polypeptide chain is as described in claim 81, wherein the antigenic protein is capable of eliciting in a subject an immune response to a coronavirus.

106. The antigenic protein of claim 105, wherein the monomeric polypeptide chain comprises a hinge, and cysteine residues in the hinge form interchain disulfide bonds between the two monomeric polypeptide chains.

107-109. (canceled)

110. A composition comprising the mRNA of claim 1 formulated in a lipid nanoparticle.

111. A vaccine comprising the mRNA of claim 1 and a pharmaceutically acceptable carrier.

112. (canceled)

113. A method for eliciting an immune response in a subject in need thereof, the method comprising: providing to the subject the vaccine of claim 111.

114. A method for preventing and/or treating a coronavirus infection in a subject in need thereof, the method comprising administering to the subject a vaccine comprising the mRNA of claim 1.

115-116. (canceled)

117. A method for preventing the occurrence of COVID-19 in a subject in need thereof, the method comprising administering to the subject a vaccine comprising an mRNA comprising an open reading frame encoding a monomeric polypeptide chain comprising a first coronavirus spike protein RBD area, a first linker, a hinge, a Fc domain, a second linker, and a second coronavirus spike protein RBD area.


SEQUENCE LISTING (292 sequences)

SEQ ID NO: 1 - DNA, 36 bp, unidentified
  source 1..36: mol_type = other DNA
atgtttgtttttcttgttttattgccactagtctct

SEQ ID NO: 2 - DNA, 48 bp, unidentified
  source 1..48: mol_type = other DNA
atgaagaccatcatcgccctgagctacatcttctgcctggtgttcgcc

SEQ ID NO: 3 - DNA, 54 bp, unidentified
  source 1..54: mol_type = other DNA
atgcacagctcagcactgctctgttgcctggtcctcctgactggggtgagggcc

SEQ ID NO: 4 - DNA, 36 bp, unidentified
  source 1..36: mol_type = other DNA
atgttcgtcttcctggtgctgctgccactggtcagc

SEQ ID NO: 5 - DNA, 36 bp, unidentified
  source 1..36: mol_type = other DNA
atgttcgtgttcctggtgctgctacccctggtgtct

SEQ ID NO: 6 - DNA, 36 bp, unidentified
  source 1..36: mol_type = other DNA
atgttcgtgttcctggtgctgctgccactggtgagc

SEQ ID NO: 7 - DNA, 36 bp, unidentified
  source 1..36: mol_type = other DNA
atgttcgtgttcctggtgctgctgccactggtcagc

SEQ ID NO: 8 - DNA, 36 bp, unidentified
  source 1..36: mol_type = other DNA
atgttcgtgtttttggtactgcttcctttggtctcc

SEQ ID NO: 9 - DNA, 36 bp, unidentified
  source 1..36: mol_type = other DNA
atgttcgtgttcctggtcctgctgccactggtgagc

SEQ ID NO: 10 - DNA, 36 bp, unidentified
  source 1..36: mol_type = other DNA
atgttcgttttcctggttttgttgccactggttagt

SEQ ID NO: 11 - DNA, 36 bp, unidentified
  source 1..36: mol_type = other DNA
atgttcgtgttcttagtgctgctgcctctggtgagc

SEQ ID NO: 12 - DNA, 36 bp, unidentified
  source 1..36: mol_type = other DNA
atgttcgtgttcctggtgctgctgcccctggtcagt

SEQ ID NO: 13 - DNA, 36 bp, unidentified
  source 1..36: mol_type = other DNA
atgttcgtgttcctggtgctgctccctctggtgagc

SEQ ID NO: 14 - DNA, 36 bp, unidentified
  source 1..36: mol_type = other DNA
atgttcgtgtttctcgttctgctgccactggtttcc

SEQ ID NO: 15 - DNA, 48 bp, unidentified
  source 1..48: mol_type = other DNA
atgttcgtgttcctggtgctgctgcccctggtgagcagccagtgcgtg

SEQ ID NO: 16 - DNA, 18 bp, synthetic construct
  source 1..18: mol_type = other DNA
aagccaactgagttggag

SEQ ID NO: 17 - DNA, 19 bp, unidentified
  source 1..19: mol_type = other DNA
taatacgactcactatagg

SEQ ID NO: 18 - skipped

SEQ ID NO: 19 - DNA, 110 bp, unidentified
  source 1..110: mol_type = other DNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 20 - DNA, 56 bp, unidentified
  source 1..56: mol_type = other DNA
gagagaataaactagtattcttctggtccccacagactcagagagaacccgccacc

SEQ ID NO: 21 - DNA, 55 bp, unidentified
  source 1..55: mol_type = other DNA
ggagaataaactagtattcttctggtccccacagactcagagagaacccgccacc

SEQ ID NO: 22 - DNA, 88 bp, unidentified
  source 1..88: mol_type = other DNA
ggaattccccgcctcctcctgtcccgcagtcggcgtccagcggctctgcttgttcgtgtgtgtgtcgttgcaggccttattcgccacc

SEQ ID NO: 23 - DNA, 56 bp, unidentified
  source 1..56: mol_type = other DNA
ggagtctctcctcataaatcttctggtccccacagactcagagagaacccgccacc

SEQ ID NO: 24 - DNA, 295 bp, unidentified
  source 1..295: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 25 - DNA, 139 bp, unidentified
  source 1..139: mol_type = other DNA
caagcacgcagcaatgcagctcaaaacgcttagcctagccacacccccacgggaaacagcagtgattaacctttagcaataaacgaaagtttaactaagctatactaaccccagggttggtcaatttcgtgccagccac

SEQ ID NO: 26 - DNA, 284 bp, unidentified
  source 1..284: mol_type = other DNA
gagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagcccaactactaaactgggggatattatgaagggccttgagcatctggattctgcctaataaaaaacatttattttcattgctgcgtc

SEQ ID NO: 27 - skipped

SEQ ID NO: 28 - DNA, 21 bp, unidentified
  source 1..21: mol_type = other DNA
gagaacctgtacttccagggc

SEQ ID NO: 29 - DNA, 30 bp, unidentified
  source 1..30: mol_type = other DNA
ggtggaggcggttcaggcggaggtggctct

SEQ ID NO: 30 - DNA, 30 bp, unidentified
  source 1..30: mol_type = other DNA
ggtggaggcggttcaggcggtggcggatcg

SEQ ID NO: 31 - DNA, 30 bp, unidentified
  source 1..30: mol_type = other DNA
ggcggtggcggatcgggaggcggtggctct

SEQ ID NO: 32 - DNA, 30 bp, unidentified
  source 1..30: mol_type = other DNA
ggcggcggaggcagcggcggcggaggcagc

SEQ ID NO: 33 - DNA, 30 bp, unidentified
  source 1..30: mol_type = other DNA
ggcggaggcggctctggcggcggaggcagc

SEQ ID NO: 34 - DNA, 30 bp, unidentified
  source 1..30: mol_type = other DNA
ggcgggggaggcagcggcggcggcggaagc

SEQ ID NO: 35 - DNA, 30 bp, unidentified
  source 1..30: mol_type = other DNA
ggcggaggcggcagcggcggcggaggcagc

SEQ ID NO: 36 - DNA, 30 bp, unidentified
  source 1..30: mol_type = other DNA
ggcggcggcggctccggaggtggcgggagt

SEQ ID NO: 37 - DNA, 30 bp, unidentified
  source 1..30: mol_type = other DNA
ggcggcggaggaagcggaggcggcggctct

SEQ ID NO: 38 - DNA, 30 bp, unidentified
  source 1..30: mol_type = other DNA
ggaggcggtggtagtgggggcggtggctca

SEQ ID NO: 39 - DNA, 30 bp, unidentified
  source 1..30: mol_type = other DNA
ggcggtggcggcagtggtggcggcggcagc

SEQ ID NO: 40 - DNA, 30 bp, unidentified
  source 1..30: mol_type = other DNA
ggtggcgggggttccgggggaggcgggagc

SEQ ID NO: 41 - DNA, 30 bp, unidentified
  source 1..30: mol_type = other DNA
ggaggaggcggctccgggggcggtggcagc

SEQ ID NO: 42 - DNA, 30 bp, unidentified
  source 1..30: mol_type = other DNA
ggcggcggcgggagcgggggaggcggctcc

SEQ ID NO: 43 - DNA, 30 bp, unidentified
  source 1..30: mol_type = other DNA
ggcggcggcgggagcggcgggggaggtagc

SEQ ID NO: 44 - DNA, 45 bp, unidentified
  source 1..45: mol_type = other DNA
ggtggaggcggttcaggcggaggtggctctggcggtggcggatcg

SEQ ID NO: 45 - DNA, 45 bp, unidentified
  source 1..45: mol_type = other DNA
ggcggtggcggatcgggtggaggcggttcaggcggaggtggctct

SEQ ID NO: 46 - DNA, 45 bp, unidentified
  source 1..45: mol_type = other DNA
ggaggaggcggatccggaggaggaggatccggcggaggcggaagc

SEQ ID NO: 47 - DNA, 45 bp, unidentified
  source 1..45: mol_type = other DNA
ggcggaggtggctctggcggtggcggatcgggaggcggtggctct

SEQ ID NO: 48 - DNA, 45 bp, unidentified
  source 1..45: mol_type = other DNA
ggcggcggaggatccggcggcggcggcagcggaggcggtgggagc

SEQ ID NO: 49 - DNA, 45 bp, unidentified
  source 1..45: mol_type = other DNA
ggcggaggcggtagcggcggcggcggttctggcggtgggggcagc

SEQ ID NO: 50 - DNA, 45 bp, unidentified
  source 1..45: mol_type = other DNA
ggcggcggcggctccggcggcggcggtagcggaggtggcgggagt

SEQ ID NO: 51 - DNA, 45 bp, unidentified
  source 1..45: mol_type = other DNA
ggcggtggaggttctgggggtggaggctctggagggggtggcagt

SEQ ID NO: 52 - DNA, 45 bp, unidentified
  source 1..45: mol_type = other DNA
ggggggggcggatctggaggcggcggctccggcggcggcggcagc

SEQ ID NO: 53 - DNA, 60 bp, unidentified
  source 1..60: mol_type = other DNA
ggtggaggcggttcaggcggaggtggctctggcggtggcggatcgggaggcggtggctct

SEQ ID NO: 54 - DNA, 60 bp, unidentified
  source 1..60: mol_type = other DNA
ggtggaggcggttcaggaggcggtggctctggcggtggcggatcgggcggaggtggctct

SEQ ID NO: 55 - DNA, 60 bp, unidentified
  source 1..60: mol_type = other DNA
ggaggaggaggcagcggaggaggcggatccggaggaggaggatccggcggaggcggaagc

SEQ ID NO: 56 - DNA, 60 bp, unidentified
  source 1..60: mol_type = other DNA
ggcggaggaggaagcggcggcggcggcagcggcggcggagggtctggaggcggcggcagc

SEQ ID NO: 57 - DNA, 60 bp, unidentified
  source 1..60: mol_type = other DNA
ggcggaggaggtagcggcggaggcggcagcggcggcggcggaagcggcggcggaggctcc

SEQ ID NO: 58 - DNA, 60 bp, unidentified
  source 1..60: mol_type = other DNA
ggcgggggcggcagcggaggaggtggctccggaggcggcggcagcggcggaggcggctct

SEQ ID NO: 59 - DNA, 60 bp, unidentified
  source 1..60: mol_type = other DNA
ggcggcggtggcagcggtggcggcggcagcggcggtggaggtagcggcggaggaggcagc

SEQ ID NO: 60 - DNA, 60 bp, unidentified
  source 1..60: mol_type = other DNA
ggcggcggcggctccggcggcggcggtagcggaggtggcgggagtggcggcggcggctct

SEQ ID NO: 61 - DNA, 60 bp, unidentified
  source 1..60: mol_type = other DNA
ggcggcggaggaagcggcggcggcggcagcggaggcggcggcagcggaggcggcggctct

SEQ ID NO: 62 - DNA, 60 bp, unidentified
  source 1..60: mol_type = other DNA
ggtggcggcggatccggaggcggcggatctggcggcggcggcagcggcggaggcggatca

SEQ ID NO: 63 - DNA, 60 bp, unidentified
  source 1..60: mol_type = other DNA
ggcggcggcggctcaggcggaggcggctccggcggaggcggcagcggcggcgggggcagc

SEQ ID NO: 64 - DNA, 57 bp, unidentified
  source 1..57: mol_type = other DNA
ggaggcggttcaggcggaggtggctctggcggtggcggatcgggaggcggtggctct

SEQ ID NO: 65 - DNA, 111 bp, unidentified
  source 1..111: mol_type = other DNA
gagagcaagtacggccccccttgcccaccctgtccagctcctgaggctgctggaggaccatccgtgttcctgtttcctccaaagcctaaggacaccctgatgatcagccgg

SEQ ID NO: 66 - DNA, 111 bp, unidentified
  source 1..111: mol_type = other DNA
gagagcaagtacggccccccttgcccaccctgtccagctcctgagttcgaaggaggaccatccgtgttcctgtttcctccaaagcctaaggacaccctgatgatcagccgg

SEQ ID NO: 67 - DNA, 105 bp, unidentified
  source 1..105: mol_type = other DNA
gacaaaactcacacatgcccaccgtgcccagcacctgaactcctggggggaccgtcagtcttcctcttccccccaaaacccaaggacaccctcatgatctcccgg

SEQ ID NO: 68 - DNA, 120 bp, unidentified
  source 1..120: mol_type = other DNA
gagcccaaatcttgtgacaaaactcacacatgcccaccgtgcccagcacctgaagcagaagggggaccgtcagtcttcctcttccccccaaaacccaaggaccagctcatgatctcccgg

SEQ ID NO: 69 - DNA, 117 bp, unidentified
  source 1..117: mol_type = other DNA
cccaaatcttgtgacaaaactcacacatgcccaccgtgcccagcacctgaactcctggggggaccgtcagtcttcctcttccccccaaaacccaaggacaccctcatgatctcccgg

SEQ ID NO: 70 - DNA, 105 bp, unidentified
  source 1..105: mol_type = other DNA
gacaaaactcacacatgcccaccgtgcccagcacctgaagccgctgggggaccgtcagtcttcctcttccccccaaaacccaaggacaccctcatgatctcccgg

SEQ ID NO: 71 - DNA, 111 bp, unidentified
  source 1..111: mol_type = other DNA
gagagcaagtacggccccccttgcccaccctgtccagctcctgaggctgctggaggaccatccgtgttcctgtttcctccaaagcctaaggacaccctgatggctagccgg

SEQ ID NO: 72 - DNA, 111 bp, unidentified
  source 1..111: mol_type = other DNA
gagtccaaatacggccctccatgccccccctgtcctgcccctgaagccgctggcggcccaagcgtgttcctgtttcccccaaagcccaaggacaccctgatgattagccgg

SEQ ID NO: 73 - DNA, 111 bp, unidentified
  source 1..111: mol_type = other DNA
gagagcaagtacgggcctccttgtcctccttgccccgcccctgaagccgccggcggaccttctgtgttcctgttcccccctaagcccaaggacaccctgatgatctctaga

SEQ ID NO: 74 - DNA, 111 bp, unidentified
  source 1..111: mol_type = other DNA
gagagcaaatacggcccaccatgtcccccctgtcctgctcccgaggccgccggcggccccagcgttttcctgttcccccctaagcccaaggacaccctgatgatctcccgg

SEQ ID NO: 75 - DNA, 111 bp, unidentified
  source 1..111: mol_type = other DNA
gagagcaagtacggccctccttgccctccttgtcccgctccagaagccgctggaggccctagcgtgttcctgtttccacccaaaccaaaggacaccctgatgatcagcaga

SEQ ID NO: 76 - DNA, 111 bp, unidentified
  source 1..111: mol_type = other DNA
gagagcaaatacggacctccatgcccaccttgccccgcccctgaggccgccggcggtcctagtgtgttcctgttcccgcctaagcctaaggacaccctgatgatcagcaga

SEQ ID NO: 77 - DNA, 111 bp, unidentified
  source 1..111: mol_type = other DNA
gaaagcaagtacggacctccttgccctccatgtcccgccccagaggctgctggcggcccttctgtgttcctgttcccccccaagcctaaggacaccctgatgatcagccgg

SEQ ID NO: 78 - DNA, 111 bp, unidentified
  source 1..111: mol_type = other DNA
gaatccaaatatgggcccccgtgtccaccttgtcctgcaccggaggcagccggcggtccttctgtgtttctcttccccccgaagccgaaggacaccctcatgatttccagg

SEQ ID NO: 79 - DNA, 111 bp, unidentified
  source 1..111: mol_type = other DNA
gaatccaagtacgggccaccttgtccgccgtgtccagcacccgaagcagcggggggaccctctgttttcctgttccccccgaagccaaaagatacgttgatgatatccagg

SEQ ID NO: 80 - DNA, 111 bp, unidentified
  source 1..111: mol_type = other DNA
gagtccaaatacggcccaccttgccctccctgccctgctcccgaggctgccggaggcccctccgtgttcctgttcccacccaagcccaaggacaccctgatgatctctagg

SEQ ID NO: 81 - DNA, 111 bp, unidentified
  source 1..111: mol_type = other DNA
gaatccaagtacggacccccttgtccaccctgccctgcccccgaggccgctggcggcccctccgtgtttctgttcccccccaagcccaaggacaccctgatgatcagccgc

SEQ ID NO: 82 - DNA, 111 bp, unidentified
  source 1..111: mol_type = other DNA
gaaagcaagtacggccctccctgccctccgtgtcccgctcctgaagccgccggcggacctagcgtgttcctgttccctcccaagcctaaggacacactgatgatcagtcgg

SEQ ID NO: 83 - DNA, 573 bp, unidentified
  source 1..573: mol_type = other DNA
acacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggc

SEQ ID NO: 84 - DNA, 576 bp, unidentified
  source 1..576: mol_type = other DNA
acccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 85 - DNA, 576 bp, unidentified
  source 1..576: mol_type = other DNA
acccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctccatgaggctctgcacaaccactacacgcagaagagcctctccctgtccccgggtaaa

SEQ ID NO: 86 - DNA, 576 bp, unidentified
  source 1..576: mol_type = other DNA
acccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - DNA, 573 bp, unidentified
  source 1..573: mol_type = other DNA
acacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggc

SEQ ID NO: 88 - DNA, 573 bp, unidentified
  source 1..573: mol_type = other DNA
acacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgaagcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggc

SEQ ID NO: 89 - DNA, 573 bp, unidentified
  source 1..573: mol_type = other DNA
acacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgatgcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggc

SEQ ID NO: 90 - DNA, 573 bp, unidentified
  source 1..573: mol_type = other DNA
acacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcagtatacccagaagtccctgagcctgtctctgggc

SEQ ID NO: 91 - DNA, 573 bp, unidentified
  source 1..573: mol_type = other DNA
acccctgaggtgacctgtgtggtggtggacgtgtcccaggaggaccccgaagtgcagttcaactggtacgtcgacggagtggaagtgcacaacgccaagaccaagcctagagaagagcagttcaattcaacatacagagtggtgagcgtgctgaccgtgctgcaccaggactggctgaacggcaaggaatacaagtgcaaggtgagcaacaagggcctgcctagcagcatcgagaagaccatcagcaaggccaaaggccagcctagagagcctcaggtgtacaccctgcctcccagccaggaggaaatgaccaaaaaccaggtgtccctgacatgcctggtcaagggcttctacccttctgatatcgccgtggaatgggagagcaacggccagcctgagaacaactacaaaacaacccctcctgtgctcgatagcgacggcagtttcttcctctactctcggctgacagtggataagagcagatggcaggagggcaacgtgttttcttgtagcgtgatgcacgaggccctgcacaaccactacacccagaagtctctgtctctgagcctgggc

SEQ ID NO: 92 - DNA, 573 bp, unidentified
  source 1..573: mol_type = other DNA
acacccgaggtgacctgcgtggtggtggacgtgtcccaggaggaccccgaggttcagtttaactggtatgtggacggcgtggaggtgcacaacgctaaaaccaagcccagagaagagcagttcaacagcacctaccgggtggtgagcgtgctgaccgtgctgcaccaggactggctgaacggcaaggaatacaaatgtaaagtgagcaacaagggcctgccttcttccatcgagaagaccatcagcaaggccaagggccagcctagagagcctcaggtgtacaccctgcccccaagccaggaggaaatgaccaaaaaccaagtgtccctgacctgtctggtgaagggattctaccctagcgacatcgccgtggaatgggaaagcaacggccagcccgaaaacaactacaaaaccacccctcctgtgttggattctgatggcagcttcttcctgtacagcagactcaccgtggacaagtctagatggcaggagggcaacgtgttctcatgctccgtgatgcacgaggccctgcacaatcactacacacagaagagcctgagcctgagcctgggc

SEQ ID NO: 93 - DNA, 573 bp, unidentified
  source 1..573: mol_type = other DNA
acccctgaagtcacatgcgtggtggtggacgtgtcccaggaggaccccgaggtgcagttcaattggtacgtggacggagtggaagtgcacaacgctaagacaaaacctagggaagaacagttcaacagcacatacagagtggttagcgtactcacagtgctgcaccaggactggctgaatggaaaggaatacaagtgcaaggtgtccaacaaggggctgccttcaagcatcgagaagaccatctctaaagccaagggccagcccagagagcctcaggtgtacaccctgcctcctagccaggaggaaatgaccaaaaaccaggttagcctgacctgcctggtgaaaggcttctacccttctgatatcgccgtggaatgggagagcaacggacaacctgagaacaactacaagaccacccctcctgtgctggacagcgacggctctttcttcctctactccagactcaccgtggacaagtctagatggcaggagggcaatgtgttctcatgcagcgtcatgcacgaggccctgcataatcactacacacagaagagcctgagcctgagcctcgga

SEQ ID NO: 94 - DNA, 573 bp, unidentified
  source 1..573: mol_type = other DNA
acacctgaagtgacctgtgtggtggtggacgtgagccaggaggaccccgaggttcaatttaattggtacgtggatggcgtggaagtgcacaatgccaaaacaaagcccagagaggaacagttcaacagcacataccgagtggtgagcgtgctgacagtgcttgcccaggactggctgaacggaaaagagtacaagtgtaaagtgagcaataaaggcctgcccagcagcatcgagaagacaatcagcaaggccaagggccagcctagagaaccccaggtgtacaccctgcctccatctcaggaggaaatgacaaagaaccaggtgtccctgacctgcctggtgaagggcttctacccctctgacatcgccgtggaatgggagagcaacggccagcctgagaacaactacaagaccacaccccctgtgctggacagcgacggcagctttttcctgtacagccgcctgaccgtggacaagtccagatggcaggagggcaacgtgttctcatgcagtgtgatgcacgaggccctgcacaaccactacacccagaaatctctgtccctgagcctgggc

SEQ ID NO: 95 - DNA, 573 bp, unidentified
  source 1..573: mol_type = other DNA
acacctgaggtgacctgtgtggtggtggacgtgtcccaggaggaccctgaagtgcagttcaactggtatgtggatggcgttgaagtgcacaacgctaagaccaagcccagagaggaacagtttaacagcacctaccgcgtcgttagcgtgctgacagtgctggcccaggactggctgaacgggaaggaatacaagtgtaaagtgtccaacaagggcctgccttctagcatcgagaagaccatcagcaaggccaagggccagcccagagagcctcaagtgtacaccctgccacctagccaggaggaaatgaccaaaaaccaggtgtctctcacctgcctcgtgaagggcttttacccctcagatatcgccgtggaatgggagagcaatggccaacccgaaaacaactacaaaaccacaccccccgtgctggacagcgacggcagctttttcctgtactccagactgaccgtggacaagtccagatggcaggagggcaacgtgtttagctgcagcgtcatgcacgaggccctgcacaaccactacacacagaagtctctgtctctgtccctggga

SEQ ID NO: 96 - DNA, 573 bp, unidentified
  source 1..573: mol_type = other DNA
acccctgaagtcacatgcgtggtggtggacgtgtcccaggaggaccccgaggtgcagttcaattggtacgtggacggagtggaagtgcacaacgctaagacaaaacctagggaagaacagttcaacagcacatacagagtggttagcgtactcacagtgctggctcaggactggctgaatggaaaggaatacaagtgcaaggtgtccaacaaggggctgccttcaagcatcgagaagaccatctctaaagccaagggccagcccagagagcctcaggtgtacaccctgcctcctagccaggaggaaatgaccaaaaaccaggttagcctgacctgcctggtgaaaggcttctacccttctgatatcgccgtggaatgggagagcaacggacaacctgagaacaactacaagaccacccctcctgtgctggacagcgacggctctttcttcctctactccagactcaccgtggacaagtctagatggcaggagggcaatgtgttctcatgcagcgtcatgcacgaggccctgcataatcactacacacagaagagcctgagcctgagcctcgga

SEQ ID NO: 97 - DNA, 573 bp, unidentified
  source 1..573: mol_type = other DNA
accccggaggttacttgtgtcgtcgtggacgtgtcacaggaagacccggaagtgcaattcaactggtacgtcgatggtgtggaggttcataacgctaaaacaaaacctcgcgaagaacagttcaacagcacgtatagggttgtaagcgtgttgacagttcttgcgcaagactggcttaatgggaaggaatacaagtgtaaagtatctaataagggactgcctagttccatcgagaaaactatttccaaagctaaggggcaaccccgggagccccaagtttacacgctccctccgtcacaggaggaaatgacaaaaaaccaggtcagtttgacatgtctggtcaagggcttctacccgtctgatatagctgtcgaatgggaatcaaacgggcaacctgaaaataactataagactacaccgcctgttctcgatagtgacgggtccttctttctgtatagcagactcactgtcgacaaatcccgctggcaagaggggaatgtgttttcctgtagcgttatgcacgaggccctccataaccattatacccaaaaatcactgtccttgagcctcgga

SEQ ID NO: 98 - DNA, 573 bp, unidentified
  source 1..573: mol_type = other DNA
acccctgaggtgacttgcgtggtagtggacgtgtcccaggaagatcctgaggtgcagttcaactggtacgtggacggcgttgaagtccacaacgccaaaaccaagccaagagaggagcagttcaacagcacctacagagtggtgagcgtgctaacagtgctggcccaggactggctgaacggaaaagagtacaagtgcaaggtgtcaaacaagggcctgcctagctctatcgagaagaccatcagcaaggccaagggccagcccagagagcctcaagtgtataccctgccccccagccaggaggaaatgaccaagaaccaggtgagcctgacctgtctggtgaagggattctacccctccgatatcgccgtggaatgggaaagcaacggacagcctgaaaacaactacaaaaccacccctcctgtgctggactccgacggcagcttcttcctgtatagcagactgaccgtggataagtccagatggcaggagggcaacgttttcagctgcagcgtgatgcacgaggccctgcataaccactacacccagaaatccctgagcctgagcctgggc

SEQ ID NO: 99 - DNA, 573 bp, unidentified; identical to SEQ ID NO: 96
  source 1..573: mol_type = other DNA

SEQ ID NO: 100 - DNA, 573 bp, unidentified
  source 1..573: mol_type = other DNA
acgccagaagtaacctgcgtggttgtggacgtcagccaagaggaccctgaagtccagtttaattggtacgtagacggagtggaggtacacaatgctaaaacaaagccgcgcgaggaacagttcaattccacctaccgagttgtctcagtcttgactgttctcgctcaggactggttgaatggaaaagaatacaagtgtaaagtttccaacaaagggcttccctcatctattgagaagacaatatctaaagcaaagggtcaacctagagaaccacaagtatatactctcccgccctcacaagaggagatgactaaaaaccaagtctctcttacgtgcctcgtgaaaggtttctacccgtctgatatcgcagtcgaatgggaaagtaacggacaacctgagaataactataagaccactccccccgtccttgacagcgatgggtctttttttctgtattccaggcttaccgttgataaaagcaggtggcaagagggcaatgtttttagctgctctgtcatgcatgaggctctccacaaccattacacccagaaaagcctgagcctgagtcttggc

SEQ ID NO: 101 - DNA, 573 bp, unidentified
  source 1..573: mol_type = other DNA
acccccgaagtgacttgcgtggtggtcgacgtgagccaggaggatccagaagtgcagttcaactggtacgtggacggcgtggaggtgcacaatgcaaagacaaaacccagggaggagcagttcaactccacataccgggtggtcagcgtgctgaccgtgctggcacaggactggctgaatggcaaggagtataagtgcaaagtgtccaacaaggggctgccaagctccatcgagaagaccatctccaaggccaagggtcagcccagggagcctcaggtgtataccctgcccccctctcaggaagagatgactaagaaccaggtgtccctgacatgcctggtgaaaggattttaccccagcgacatcgccgtggagtgggagagcaatggccagcccgaaaacaactacaagacaacccctcccgtgctggacagcgatggatcattcttcctgtacagcaggctgacagtggacaaatcacggtggcaggagggaaacgtgttcagctgctccgtgatgcacgaggccctccacaatcactacacacagaaaagcctgagcctgtcactgggc

SEQ ID NO: 102 - DNA, 573 bp, unidentified
  source 1..573: mol_type = other DNA
accccagaggtgacttgcgtggtggtggatgtgtcccaggaggacccagaggtgcagttcaactggtatgtggacggggtggaggtgcacaacgccaagaccaagcctagggaggagcagttcaatagcacttatagggtggtgtccgtgttgacagtgctggcccaggactggctgaatggcaaggagtataagtgcaaagtcagcaataagggactccctagctccatcgaaaaaaccatctccaaggctaaggggcagcccagggagccccaggtgtacaccctgccccccagccaggaggagatgaccaagaaccaggtgagtctcacttgcctggtgaagggcttttacccttccgacattgcagtggagtgggaaagcaacggacagccagaaaataactataagactacccctcccgtgctggactccgatgggtcattctttctgtacagcaggctgacagtggataagagcagatggcaggagggcaacgtcttttcctgtagcgtgatgcacgaagccctgcacaatcactacacccagaagtccctgagcctgagcctgggt

SEQ ID NO: 103 - DNA, 573 bp, unidentified
  source 1..573: mol_type = other DNA
acacctgaggtgacctgtgtggtggtggacgtgtcccaggaggaccctgaagtgcagttcaactggtatgtggatggcgttgaagtgcacaacgctaagaccaagcccagagaggaacagtttaacagcacctaccgcgtcgttagcgtgctgacagtgctggcccaggactggctgaacgggaaggaatacaagtgtaaagtgtccaacaagggcctgccttctagcatcgagaagaccatcagcaaggccaagggccagcccagagagcctcaagtgtacaccctgccacctagccaggaggaaatgaccaaaaaccaggtgtctctcacctgcctcgtgaagggcttttacccctcagatatcgccgtggaatgggagagcaatggccaacccgaaaacaactacaaaaccacaccccccgtgctggacagcgacggcagctttttcctgtactccagactgaccgtggacaagtccagatggcaggagggcaacgtgtttagctgcagcgtcatgcacgaggccctgcacaaccactacacacagaagtctctgtctctgtccctgggt

SEQ ID NO: 104 - DNA, 573 bp, unidentified; identical to SEQ ID NO: 103
  source 1..573: mol_type = other DNA

SEQ ID NO: 105 - DNA, 669 bp, unidentified
  source 1..669: mol_type = other DNA
agagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttc

SEQ ID NO: 106 - DNA, 669 bp, unidentified
  source 1..669: mol_type = other DNA
agagtgcagcccacagaaagcatcgtgcgcttccccaacatcaccaacctgtgcccattcggcgaggtattcaacgccacccggtttgcctctgtgtatgcctggaatagaaagcggatcagcaactgcgtggccgattacagcgtgctgtacaacagcgcttctttcagcaccttcaagtgctacggcgtgagccctaccaagctgaacgacctgtgcttcacaaatgtgtacgccgacagcttcgtgatcagaggcgacgaggtcagacagatcgcccctggccaaacaggaaacatcgccgactataattacaagctgcccgacgacttcaccggctgcgtgattgcttggaatagcaacaacctggacagcaaggtgggaggcaactacaactatctgtatcggctgttcagaaagagtaatctgaagcctttcgagcgggacatcagcaccgagatctaccaagccggcagcacaccctgcaacggcgtgaagggcttcaactgctacttccccctgcaaagctacggcttccagcccacctacggcgtgggctaccagccataccgggtggttgttctgtcgttcgaactactgcacgcccctgccacagtgtgcggccccaagaaatctacaaacctggtgaagaacaaatgcgtaaacttc

SEQ ID NO: 107 - DNA, 669 bp, unidentified
  source 1..669: mol_type = other DNA
agagtgcagcccaccgaaagcattgtgcggttccccaacatcacaaacctgtgccctttcggcgaggtgttcaacgccacccgctttgctagcgtttacgcctggaacagaaagagaatcagcaattgcgtggccgattacagcgtgctgtacaacagcgcctctttcagcacattcaagtgttatggcgtcagccctacaaagctgaacgacctgtgcttcaccaacgtgtacgccgacagctttgtgattagaggcgatgaggtgcgacagatcgcccctggccagaccggcaagatcgccgactacaattataagctgcctgacgatttcaccggctgtgtgatcgcctggaactctaacaacctggatagcaaggtgggaggaaactacaactaccagtacagactgttccggaagtccaacctgaagccctttgagcgggacatcagcaccgagatctaccaagctggctcaacaccttgcaacggcgttgaaggctttaactgttacagccctctgcagtcctacggtttccagcctaccaacggcgtgggctaccagccctaccgggtggtggtcctgagcttcgagctgctgcacgccccggctaccgtgtgcggccccaagaaaagcaccaacctggtgaaaaacaagtgcgtgaacttc

SEQ ID NO: 108 - DNA, 669 bp, unidentified
  source 1..669: mol_type = other DNA
agagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttc

SEQ ID NO: 109 - DNA, 666 bp, unidentified
  source 1..666: mol_type = other DNA
ggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaat

SEQ ID NO: 110 - DNA, 1125 bp, unidentified
  source 1..1125: mol_type = other DNA
acttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttc

SEQ ID NO: 111 - DNA, 657 bp, unidentified
  source 1..657: mol_type = other DNA
agagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaa

SEQ ID NO: 112 - DNA, 591 bp, unidentified
  source 1..591: mol_type = other DNA
actaatctttgtccgttcggtgaggtttttaacgcgacaaggttcgctagtgtatatgcttggaaccgaaagagaatctccaattgcgtagctgattactccgttctctataacagtgcgtccttttcaacctttaagtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtggtaattataattaccggtatcgcctgtttcgaaagagcaatttgaagccttttgagcgggatatttcaaccgaaatttaccaagcaggcagtaaaccatgtaacggagtagagggatttaattgctactttcctcttcaatcttatggctttcaaccaacaaacggagtggggtatcaaccttatagagtggtagtattgtcctttgagctcctccacgccccggctacagtttgtgggcccaaaaag

SEQ ID NO: 113 - DNA, 669 bp, unidentified
  source 1..669: mol_type = other DNA
agagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttc

SEQ ID NO: 114 - DNA, 657 bp, unidentified
  source 1..657: mol_type = other DNA
agagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataag

SEQ ID NO: 115 - DNA, 591 bp, unidentified
  source 1..591: mol_type = other DNA
acaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaag

SEQ ID NO: 116 - DNA, 669 bp, unidentified
  source 1..669: mol_type = other DNA
agagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttc

SEQ ID NO: 117 - DNA, 657 bp, unidentified
  source 1..657: mol_type = other DNA
agagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataag

SEQ ID NO: 118 - DNA, 591 bp, unidentified
  source 1..591: mol_type = other DNA
acaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaag

SEQ ID NO: 119 - DNA, 669 bp, unidentified
  source 1..669: mol_type = other DNA
agagtccagccaactgaatctattgtgcgcttcccgaacatcacaaacttgtgcccatttgatgaagtgtttaacgctacccggttcgcttccgtgtatgcttggaacagaaaacgaataagtaactgcgttgcggattactctgtgttgtataatcttgcgcccttctttacattcaagtgctacggagtatcccctacgaagcttaacgatttgtgttttacgaacgtctatgcggattcctttgtcattcgcggggacgaggtgagacagattgcacctggacaaacggggaacattgcggattataactataaacttcccgatgatttcacagggtgcgtgatagcgtggaactcaaataagcttgatagtaaggtatcaggaaattacaattacctttataggcttttccgcaaatcaaacctgaagccctttgaaagggacattagcaccgagatttatcaagccggcaataagccgtgtaatggtgttgcgggcttcaactgttactttcccctcagatcttattcctttagacctacttatggtgtaggccatcagccatacagagtagtagtccttagttttgagttgctgcatgcaccggcaacagtttgtggccctaagaagtcaacaaatctggtgaaaaataagtgcgtcaatttc

SEQ ID NO: 120 - DNA, 669 bp, unidentified
  source 1..669: mol_type = other DNA
agggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtccaccaacctggtgaagaacaaatgcgtgaacttc

SEQ ID NO: 121 - DNA, 657 bp, unidentified
  source 1..657: mol_type = other DNA
agggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtccaccaacctggtgaagaacaaa

SEQ ID NO: 122 - DNA, 720 bp, unidentified
  source 1..720: mol_type = other DNA
agggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtccaccaacctggtgaagaacaaatgcgtgaacttcaacttcaatggtttaaaaggcacaggtgttcttactgagtctaacaaaaag

SEQ ID NO: 123 - DNA, 633 bp, unidentified
  source 1..633: mol_type = other DNA
agggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaag

SEQ ID NO: 124 - DNA, 600 bp, unidentified
  source 1..600: mol_type = other DNA
ccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaag

SEQ ID NO: 125 - DNA, 576 bp, unidentified
  source 1..576: mol_type = other DNA
agggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtg

SEQ ID NO: 126 - DNA, 600 bp, unidentified
  source 1..600: mol_type = other DNA
ccaaacatcacaaacctgtgcccattcgacgaggtgttcaacgcaaccaaattcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaag

SEQ ID NO: 127 - DNA, 600 bp, unidentified
  source 1..600: mol_type = other DNA
ccaaacatcacaaacctgtgcccattcgacgaggtgttcaacgcaaccaggttcgcaagcgtgtacgcatggaacaggaagaggatcagcaactgcgtggcagactactctgtgctgtataatttcgcccccttttttgcttttaagtgctatggcgtgagtcccacaaagctgaacgatctgtgcttcaccaacgtgtacgccgactcctttgtgattaggggcaatgaggtgagccagatcgcccctggacagaccggaaacatcgccgactataactacaagctgcccgacgacttcaccggctgcgtgatcgcctggaactccaacaagctggactccaaagtgggtggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctacggtttccgccccacctacggcgtgggacaccagccttaccgggtggtggtgctgtccttcgaactgctgcacgcacccgccaccgtgtgcggacctaaaaag

SEQ ID NO: 128 - DNA, 657 bp, unidentified
  source 1..657: mol_type = other DNA
agggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatttcgcccccttttttgcttttaagtgctatggcgtgagtcccacaaagctgaacgatctgtgcttcaccaacgtgtacgccgactcctttgtgattaggggcaatgaggtgagccagatcgcccctggacagaccggaaacatcgccgactataactacaagctgcccgacgacttcaccggctgcgtgatcgcctggaactccaacaagctggactccaaagtgggtggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctacggtttccgccccacctacggcgtgggacaccagccttaccgggtggtggtgctgtccttcgaactgctgcacgcacccgccaccgtgtgcggacctaaaaagtccaccaacctggtgaagaacaaa

SEQ ID NO: 129 - DNA, 669 bp, unidentified
  source 1..669: mol_type = other DNA
agagttcagccgactgagtctattgtgcggtttcctaacatcaccaacctgtgccccttcgacgaggtgtttaacgccacaagattcgccagcgtgtacgcctggaaccggaaaagaatcagcaactgcgtggccgactactctgtactgtacaacctggcccctttcttcaccttcaagtgctacggagtgtcccctaccaagctgaacgatctgtgcttcaccaacgtgtacgccgacagcttcgtgatcagaggagatgaagtgcggcaaatcgcacccggccagacaggcaatattgccgattataactacaagctgcccgacgacttcaccggatgcgtgattgcttggaacagcaacaagctggactccaaggtttctggcaactacaactacctgtacagactgttcagaaagtccaacctgaagcctttcgagagagatatctctaccgagatctaccaagccggaaacaagccctgtaatggcgttgctggctttaactgctacttccccctgagatcttacagcttcagacctacctacggcgtgggccaccagccttaccgggtcgtggtgctgtctttcgaactgcttcacgccccagccacagtgtgcggccctaagaagtctaccaatctggtgaagaacaagtgtgtgaacttc

SEQ ID NO: 130 - DNA, 669 bp, unidentified
  source 1..669: mol_type = other DNA
agagtgcagcctaccgaatctatcgtgcggttccctaacatcaccaatctgtgcccctttggagaggtgttcaatgcgactcggttcgcttctgtgtacgcttggaacagaaagcggatcagcaactgcgtcgccgactacagcgtgctgtacaacagcgccagctttagcacctttaagtgctacggcgtgtctcctaccaagctgaatgatctgtgtttcaccaacgtgtacgccgacagctttgtcatccgtggagatgaggtgagacagatcgcccctggccagaccggcaaaatcgccgattacaactacaagctgcccgatgacttcaccggatgtgtgatcgcctggaactctaacaacctggactccaaggtgggcgggaattataactacagatacagactgttcagaaagagcaatctcaagcctttcgagagagatatttccacagaaatctaccaggccggcagcaaaccttgtaacggcgttgaaggctttaattgctacttccctctgcagagctacggcttccaacctacaaacggcgtgggctatcagccgtacagagtcgtggtcctgtcctttgagctgcttcatgcccctgctaccgtgtgcggtcctaaaaagagtacaaacctggtgaagaacaagtgtgtgaacttc

SEQ ID NO: 131 - DNA, 657 bp, unidentified
  source 1..657: mol_type = other DNA
agagtccagcctacagaatctatcgtgagattccctaacatcaccaacctgtgccctttcggcgaggtctttaacgccacaagattcgcctccgtgtacgcctggaatagaaagcggatcagcaactgcgttgctgattatagcgtcctgtacaactctgctagcttcagcaccttcaagtgctacggcgtgagccccacaaagctgaatgacctgtgttttaccaacgtgtatgctgactctttcgtaatccggggcgacgaggttcggcagatcgctccaggccagaccggcaagatcgccgattacaactacaagctgcccgacgacttcacaggatgtgtgattgcctggaacagcaacaacctggacagcaaggtgggcggcaattacaactacagataccggctgtttagaaagtccaacctgaagcctttcgagagagacatcagcaccgaaatctaccaagctggcagcaaaccttgtaatggagtggaaggcttcaactgctatttccctctgcaatcttatggcttccagcccaccaacggcgtgggataccagccttaccgcgtggtggtgctgagcttcgagctgctgcatgccccagccacagtgtgtggccctaagaagtccactaacctggtgaagaacaaa

SEQ ID NO: 132 - DNA, 669 bp, unidentified
  source 1..669: mol_type = other DNA
agagtgcagcccaccgaaagcatcgtgcgcttccctaatatcaccaatttgtgcccatttggcgaggtgttcaacgccacacggtttgcctccgtgtacgcctggaaccggaagagaatctctaattgcgtggccgactacagcgttctgtacaacagcgcctctttctctacttttaagtgctacggcgtctctcctacaaagctgaacgacctgtgcttcaccaacgtgtacgccgacagctttgtgatcaggggcgatgaggtccggcagatcgcccctggccaaacaggaaacattgccgactacaattacaagctgccagatgatttcaccggctgtgtgatcgcctggaattcgaacaacctggatagcaaagtgggcggcaactacaattacctgtacagactgttcagaaagagcaatctgaagcccttcgaaagagatatcagcacagagatctaccaggccggaagcacaccttgcaacggcgtgaaaggattcaactgctacttcccactgcagagctacggcttccagcctacctacggcgtcggctaccagccttaccgagtggtcgtgctgagctttgagctgctgcacgcccctgctacagtgtgcggccccaagaaaagcaccaacctggtcaagaacaagtgcgtgaacttc

SEQ ID NO: 133 - DNA, 657 bp, unidentified
  source 1..657: mol_type = other DNA
cgggtccagcctaccgagagtatcgtgagattccctaatatcaccaacctgtgtcctttcggcgaagtgttcaacgccacaagattcgccagcgtgtacgcctggaatagaaagagaatctccaattgcgtggccgactacagcgtgctgtacaactctgccagcttttctacatttaagtgctacggcgtgtctcctaccaagctgaatgatctgtgctttacaaacgtgtacgccgacagcttcgtgatcagaggcgacgaggtgcgccagatcgcccctggacaaactggcaagatcgccgattacaactacaagctgcctgatgacttcaccggctgcgtgatcgcatggaacagcaacaacctggacagcaaggtgggcggcaactacaactaccggtacagactgtttcggaagagcaacctgaaacctttcgagagagatatctccaccgagatctaccaggccggatccaagccgtgcaatggcgtggaaggcttcaactgctacttcccactgcaatcttatggcttccagccaaccaacggcgttggatatcagccctatcgggtagtggtgttgtcttttgagctgctgcacgcccctgccacagtgtgtggacccaagaagagcaccaacctggtcaagaacaaa

SEQ ID NO: 134 - DNA, 669 bp, unidentified; identical to SEQ ID NO: 129
  source 1..669: mol_type = other DNA

SEQ ID NO: 135 - DNA, 669 bp, unidentified
  source 1..669: mol_type = other DNA
agagtgcagcctaccgagagcatcgtgcggttccccaatatcaccaacctgtgcccttttggcgaggtgtttaacgccaccagattcgcctctgtgtatgcttggaatagaaagagaatcagcaactgcgtcgctgattactccgtcctctataattctgccagcttcagcaccttcaagtgctacggtgtcagccctaccaagctgaatgacctgtgttttaccaatgtctacgccgatagcttcgtgatccggggcgacgaggtgcggcagatcgcccctggccaaacaggcaagatcgccgattacaactacaagctgcctgatgacttcacaggctgcgtgatcgcttggaacagcaacaacctggatagcaaggtgggaggcaactacaactaccggtacagactcttccggaagtctaacctgaagcctttcgaacgggacatcagcaccgaaatctaccaggccggcagcaagccttgcaacggcgtggaaggcttcaattgctatttccctctgcagagctatggcttccagccaaccaacggcgtgggctaccagccttacagggtggtggtgttgtctttcgagctgctgcatgcccctgccaccgtttgtggcccaaagaaaagcaccaacctggttaagaacaagtgcgtgaacttc

SEQ ID NO: 136 - DNA, 669 bp, unidentified
  source 1..669: mol_type = other DNA
cgggtgcagcctactgaatctatcgtgcggttcccaaacatcaccaacctgtgcccttttggagaagtgttcaacgccacaaaattcgcatctgtgtatgcctggaatagaaagcggatttctaattgcgtggctgattattctgtgctgtataatagcgccagcttcagcacctttaagtgctacggcgtgtcccctaccaagctgaacgacctgtgcttcaccaacgtatacgccgatagctttgtgattagaggcgacgaggtccgtcagatcgctcctggccaaacaggcaaaatcgctgattacaactacaagctgcctgacgacttcaccggctgcgtgatcgcctggaacagcaataacctagacagcaaagtgggcggaaactacaactacctgtaccggctgttcagaaagagcaacttaaagcctttcgaaagagacatcagcaccgagatctaccaggccggatctaccccctgtaatggcgtgaagggcttcaactgctacttccctctgcagtcttatggcttccagccaacctacggcgtgggctaccagccctatagagtggtggtgctgtcctttgagctgctgcacgcccctgccaccgtgtgcggacctaagaaatctacaaacctggtgaagaacaaatgcgtgaacttc

SEQ ID NO: 137 - DNA, 669 bp, unidentified
  source 1..669: mol_type = other DNA
agagtgcagcccacagaaagcatcgtgcggttccctaacatcacaaatctgtgtcctttcggcgaggtgttcaatgccacaagattcgccagcgtctacgcctggaacagaaagagaatcagcaactgcgtcgccgactactctgtgctgtacaactctgctagcttcagcaccttcaagtgctacggcgtgagccctaccaagctgaacgacctgtgtttcaccaatgtgtacgccgacagcttcgttatcagaggagatgaggtgcggcagatcgccccaggccagaccggcaagatcgctgattacaactacaagctgcccgatgatttcacaggctgtgtgatcgcctggaactccaacaacctggacagcaaggtgggcggcaactacaactacagataccggctgtttagaaagagcaacctgaagcctttcgagcgggacatcagcacagagatctaccaagccggcagcaaaccctgcaacggcgtggaaggcttcaactgttacttccctctccagagctacggatttcagcctaccaacggagtgggctaccagccttacagggtggtggtgctgagctttgagctgctgcatgcccctgccacagtgtgcggccccaagaagtccaccaatctggttaagaacaagtgcgtgaacttc

SEQ ID NO: 138 - DNA, 669 bp, unidentified
  source 1..669: mol_type = other DNA
agagtgcagccaacagagagcatcgtgcggtttcccaatatcaccaacctgtgcccctttggagaggtgttcaatgctaccaagttcgccagcgtgtacgcctggaacagaaagcggatcagcaattgcgtggccgactactcggtgctgtataactccgccagcttcagcacattcaagtgctacggcgtctcccccaccaaactgaacgacctgtgcttcaccaacgtgtacgccgactccttcgtgatccggggcgacgaggtgagacagatcgctcccggccagacaggaaagatcgccgattacaactacaagctccctgatgacttcacaggctgtgtgattgcatggaatagcaacaatctggatagcaaggtgggcggcaactacaactacctgtacagactgtttcggaagtctaacctgaagcctttcgagagagacatcagcacagagatctaccaggccggctccacaccttgtaatggcgtcaagggcttcaactgctactttcctcttcagagctacggattccaacctacctacggagtgggctaccagccttacagagtcgtggtgctgtctttcgaactgctgcacgcccctgctacagtgtgtggccccaagaaatctacaaacctggtgaaaaacaagtgcgtgaacttc

SEQ ID NO: 139 - DNA, 669 bp, unidentified
  source 1..669: mol_type = other DNA
cgggtgcagcctactgaatctattgtgcggttccctaatatcaccaacctgtgtcctttcggcgaggtgtttaacgccacaagatttgccagcgtgtatgcctggaaccggaagagaatcagcaactgcgtcgccgactactctgtgctgtacaacagcgcctcttttagcacatttaagtgctacggtgtctcccctaccaagctgaatgacctgtgcttcaccaacgtgtacgccgatagcttcgtgatcagaggcgatgaagtgagacagatcgcccctggccaaacaggcaagatcgccgattacaattataagctgcccgacgacttcaccggctgcgtgatcgcctggaacagtaacaatctggacagcaaggtaggcggcaactacaactatcgctacaggctgttcagaaaatctaatctgaagccatttgagcgggacatcagcaccgagatctaccaagctggcagcaagccctgcaacggcgtggaaggcttcaactgttacttccctctgcagtcttacggcttccagcctaccaacggagtgggctaccagccatacagagtggtggtgctgagcttcgagctgctgcacgcacctgccaccgtgtgcggccctaagaagtctaccaacctggtcaagaacaagtgcgtgaacttc

SEQ ID NO: 140 - DNA, 669 bp, unidentified
  source 1..669: mol_type = other DNA
cgggtccagcctaccgagagtatcgtgagattccctaatatcaccaacctgtgtcctttcggcgaagtgttcaacgccacaaaattcgccagcgtgtacgcctggaatagaaagagaatctccaattgcgtggccgactacagcgtgctgtacaactctgccagcttttctacatttaagtgctacggcgtgtctcctaccaagctgaatgatctgtgctttacaaacgtgtacgccgacagcttcgtgatcagaggcgacgaggtgcgccagatcgcccctggacaaactggcaagatcgccgattacaactacaagctgcctgatgacttcaccggctgcgtgatcgcatggaacagcaacaacctggacagcaaggtgggcggcaactacaactacctgtacagactgtttcggaagagcaacctgaaacctttcgagagagatatctccaccgagatctaccaggccggatccacaccgtgcaatggcgtgaagggcttcaactgctacttcccactgcaatcttatggcttccagccaacctacggcgttggatatcagccctatcgggtagtggtgttgtcttttgagctgctgcacgcccctgccacagtgtgtggacccaagaagagcaccaacctggtcaagaacaaatgcgtcaatttc

SEQ ID NO: 141 - DNA, 669 bp, unidentified
  source 1..669: mol_type = other DNA
agagtgcagcccaccgaaagcattgtgcggttccccaacatcacaaacctgtgccctttcggcgaggtgttcaacgccacccgctttgctagcgtttacgcctggaacagaaagagaatcagcaattgcgtggccgattacagcgtgctgtacaacagcgcctctttcagcacattcaagtgttatggcgtcagccctacaaagctgaacgacctgtgcttcaccaacgtgtacgccgacagctttgtgattagaggcgatgaggtgcgacagatcgcccctggccagaccggcaagatcgccgactacaattataagctgcctgacgatttcaccggctgtgtgatcgcctggaactctaacaacctggatagcaaggtgggaggaaactacaactaccggtacagactgttccggaagtccaacctgaagccctttgagcgggacatcagcaccgagatctaccaagctggctcaaaaccttgcaacggcgttgaaggctttaactgttactttcctctgcagtcctacggtttccagcctaccaacggcgtgggctaccagccctaccgggtggtggtcctgagcttcgagctgctgcacgccccggctaccgtgtgcggccccaagaaaagcaccaacctggtgaaaaacaagtgcgtgaacttc

SEQ ID NO: 142 - DNA, 669 bp, unidentified
  source 1..669: mol_type = other DNA
agggttcagcctaccgaatcaatcgtgagatttccaaatatcacaaacctttgtccatttggtgaagtcttcaatgcaacgaaatttgccagtgtatatgcctggaaccgcaagcggatcagtaactgtgtggctgattactccgttttgtacaatagcgcgtcttttagcaccttcaagtgttatggagtaagcccgacgaagcttaacgatttgtgcttcactaatgtatacgccgactcttttgtcataagaggagatgaagtacgacagattgcgcccgggcagacgggcaagatcgcagattataactataaactcccggacgattttacgggctgcgtaatcgcttggaattcaaacaatttggatagcaaggtaggcgggaattataattacttgtatcgactttttagaaagagtaatttgaaacctttcgaacgcgacatttctaccgagatctaccaagcgggttccactccgtgcaacggggtaaaagggttcaactgttactttcctctccaatcctacgggttccaaccaacctatggtgtggggtatcaaccatatcgagtggttgttttgtctttcgagcttctccatgcccctgctactgtgtgtggtccaaaaaaatccacaaatcttgttaagaataaatgcgtcaatttt

SEQ ID NO: 143 - DNA, 669 bp, unidentified
  source 1..669: mol_type = other DNA
agagtgcaaccaacagaaagtatagtcaggtttccaaacattactaatctgtgcccatttggagaggtattcaacgcgacgcgattcgcttccgtttacgcttggaacagaaagaggatctccaattgtgtggcagactattcagttctttataattctgctagttttagcacattcaaatgctacggagtaagtcccacaaaattgaacgatctttgctttacaaacgtctatgcagatagcttcgtcattcgcggggacgaagttcgacagatagcgccaggtcagacaggcaagattgcggactataactacaaattgccggatgacttcacaggttgtgtcatcgcatggaactctaacaatctggattccaaagtgggggggaactataactacagatacagactgttcagaaagagcaacctgaagccatttgagagggacatatcaacagaaatataccaggcgggcagtaagccatgtaacggtgttgaaggttttaattgctatttcccgctgcagagttatggtttccaaccgacgaacggggtcggataccaaccgtatagagtagtcgttttgagctttgagttgttgcatgctccagcgaccgtctgtggtcctaagaaaagcacgaacttggttaaaaataagtgcgtcaacttt

SEQ ID NO: 144 - DNA, 669 bp, unidentified
  source 1..669: mol_type = other DNA
agagtgcaaccaaccgaatctattgtgcgcttccctaatatcaccaacctttgtccattcggcgaggtctttaacgcaacgaaattcgcctctgtgtatgcttggaaccgaaagcggatcagcaactgcgtcgcggactacagcgtcctctataattctgcaagttttagtacgttcaagtgttatggcgtatcccccacgaaattgaacgacctttgcttcacgaatgtgtacgcggactcttttgtgatacggggggatgaggtccgccagatcgcgcctggacagaccgggaagatagctgattacaactacaaactccccgatgactttacgggctgtgtaatagcgtggaactcaaataatctggactcaaaggtaggcggcaattataactacctgtaccgcctctttcggaagagcaatctcaagccgtttgaaagagatatttccactgagatttaccaagcaggatctaccccctgcaatggtgtcaagggtttcaattgctatttcccgcttcagagctacgggttccagcctacttacggggtcggttaccaaccgtacagagtggtggtcctgtcatttgagctgcttcatgcacctgcaacggtgtgtggcccgaagaagagcaccaacttggttaaaaacaagtgcgtaaacttc

SEQ ID NO: 145 - DNA, 669 bp, unidentified
  source 1..669: mol_type = other DNA
agagtccaacctacggagtctatcgtccgattccccaacataaccaatctgtgcccgttcggggaagtcttcaatgctacgcgattcgcgagtgtgtatgcgtggaatcggaagcggatctctaactgtgtggcggattattcagttttgtacaacagcgcctctttcagcacttttaagtgctatggcgttagtcccacaaagcttaacgacctttgttttacgaacgtgtacgccgattctttcgtcattcggggggacgaagtccgccaaatagcaccgggtcagacgggtaagatagccgattataactacaaattgcccgacgacttcacgggatgtgtgatagcctggaactctaataatcttgacagtaaggtggggggtaattacaactacaggtatcgcttgttcaggaaaagtaatctcaagccctttgaacgagatatcagtacagaaatatatcaggcaggatccaaaccttgtaacggggttgaggggtttaactgctacttccctctgcaaagctacgggttccagccgacaaatggtgtgggctaccagccttacagagtggtagttttgagttttgaattgcttcacgcaccggcaactgtatgcggtcccaaaaagagcaccaaccttgtgaagaacaaatgcgtgaacttc

SEQ ID NO: 146 - DNA, 669 bp, unidentified
  source 1..669: mol_type = other DNA
agagtgcagcccaccgagtccatcgtgaggttccctaacattaccaatctgtgtcccttcgatgaagtgttcaatgccacccgcttcgcttctgtctacgcctggaaccgcaagaggattagcaactgcgtggccgactactccgtgctgtataacctggcccctttcttcaccttcaagtgctacggcgtgtcccccactaagctgaatgacctgtgcttcacaaacgtgtatgctgacagtttcgtcatcagaggcgacgaagtgaggcagatagcccctgggcagactgggaacatcgccgactataactacaagctgccagatgatttcacaggctgcgtgatcgcttggaacagcaataagctggatagcaaggtctctggcaactataactatctgtaccgcctgtttcggaaatctaatctgaagcccttcgagcgggacatcagcaccgaaatctaccaggctggcaataagccatgcaacggcgtggccggcttcaattgttattttcctctgagatcttacagtttcaggcctacctacggggtgggccaccagccctatcgggtggtggtgctgagcttcgagctgctgcatgcccccgccactgtgtgtgggccaaagaaatccacaaacctggtgaagaacaaatgtgtgaacttt

SEQ ID NO: 147 - DNA, 669 bp, unidentified
  source 1..669: mol_type = other DNA
agggttcagccaactgaatccatcgtgcgcttccccaacattaccaatctgtgtccattcggggaggtgttcaacgccactaggttcgccagcgtgtacgcctggaaccggaagcgcatcagcaactgcgtggccgactattccgtgctgtataactctgcctccttttccacttttaagtgctacggggtgtctccaacaaagctgaatgacctgtgctttaccaatgtgtacgccgatagcttcgtgatcaggggcgatgaagtccggcagatcgcccccgggcagacaggcaagatcgccgattataattataagctgccagacgatttcaccggctgcgtgatcgcatggaatagcaacaatctggactccaaagtgggggggaattataactaccgctaccgcctgttccgcaaatccaatctgaagccattcgagagagacatttccacagagatttatcaggccggctctaaaccctgcaacggcgtggaaggattcaattgttacttccccctgcagtcatatggcttccagcctaccaacggcgtgggctatcagccttatcgggtggtggtgctgtctttcgagctgctgcatgcacccgccacagtgtgtggcccaaagaaaagcaccaatctggtgaaaaacaagtgcgtgaacttc

SEQ ID NO: 148 - DNA, 669 bp, unidentified
  source 1..669: mol_type = other DNA
cgggtgcagcctaccgagtctatcgtgaggttccctaatatcaccaacctgtgtcctttcgacgaagtcttcaacgccaccagatttgccagcgtgtatgcctggaacaggaagagaatctctaactgcgtggccgactactctgtgctatacaacctggccccctttttcacattcaagtgctacggcgtgtcccctaccaaactgaacgacctgtgttttaccaatgtgtacgccgatagcttcgtgatcaggggcgacgaggtgcgccagattgcccctggacagactggaaacatcgccgactataattacaagctgccagacgacttcaccggctgcgtgattgcatggaattctaataaactggattccaaggtttccggcaattacaactacctgtaccgcctgttccggaagtccaacctgaagcccttcgaaagggatatcagcaccgaaatctaccaggccggcaacaagccttgcaacggggtggctgggttcaactgctacttcccactgagaagttacagcttccgccctacctacggcgtggggcaccagccctatagagttgtggtgctgagcttcgagctcctgcatgcccccgccaccgtgtgcggacccaagaaatcaaccaacctggtgaagaacaaatgtgtgaacttc

SEQ ID NO: 149 - DNA, 669 bp, unidentified
  source 1..669: mol_type = other DNA
agagtgcagcccaccgagtcaattgtgaggtttcctaacatcaccaatctgtgtccattcggcgaggtgttcaacgccactaggttcgccagcgtgtacgcctggaaccggaagcgaatttccaattgtgtggcagactacagcgtgctgtacaactccgctagcttcagcacatttaagtgctacggcgtgagccctacaaagctgaatgacctgtgtttcaccaatgtgtacgccgattccttcgtgatcaggggcgacgaggtgagacagatcgcccctggccagacagggaagattgccgactataactataagctcccggatgactttaccggatgtgtgattgcttggaactccaacaacctggacagcaaagtgggtggcaactataactatagataccggctgtttcgcaagtctaacctgaaaccttttgagcgggacatctccactgagatctaccaggccgggagcaaaccatgcaacggtgtggaaggctttaactgctacttcccgctgcagtcttacggattccagccaactaatggggtgggctaccagccatatcgcgtggtggtgctgtcctttgagctgctgcacgcccctgccaccgtgtgcggacctaaaaaaagcacaaaccttgtgaaaaacaaatgcgtgaatttc

SEQ ID NO: 150 - DNA, 669 bp, unidentified
  source 1..669: mol_type = other DNA
agggttcaacctacagagtccatcgtgcggttccctaacattaccaacctctgtcctttcggcgaggtgttcaacgccacaagattcgcctctgtgtacgcctggaacagaaaaagaatctccaattgcgtcgctgattactctgtgctctataacagcgccagcttcagcaccttcaaatgctacggagtgagccctaccaaactgaacgacctgtgcttcaccaatgtgtatgccgacagcttcgtgatcagaggcgatgaggtgcgccagatcgcccccggccaaaccggcaaaatcgccgactacaactataagctgccagacgacttcacaggatgtgtgatcgcttggaacagcaacaacctggactctaaggtgggcggcaactacaactatcggtaccggctgtttagaaagagcaacctgaagcctttcgagcgggacatcagtacagaaatctaccaggccggaagcaagccctgcaacggcgtggagggcttcaattgctactttcccctgcagagctacggctttcaacctaccaatggagtgggctaccagccttaccgggtggtggtgctgagctttgagctgctgcacgcccctgccaccgtgtgcggacctaagaaatctacaaacctggtgaagaacaagtgcgtgaacttc

SEQ ID NO: 151 - DNA, 669 bp, unidentified
  source 1..669: mol_type = other DNA
cgggtgcagcctaccgagtctatcgtgaggttccctaatatcaccaacctgtgtcctttcggcgaagtcttcaacgccaccagatttgccagcgtgtatgcctggaacaggaagagaatctctaactgcgtggccgactactctgtgctatacaacagcgcctctttttctacattcaagtgctacggcgtgtcccctaccaaactgaacgacctgtgttttaccaatgtgtacgccgatagcttcgtgatcaggggcgacgaggtgcgccagattgcccctggacagactggaaagatcgccgactataattacaagctgccagacgacttcaccggctgcgtgattgcatggaattctaataacctggattccaaggttggcggcaattacaactaccggtaccgcctgttccggaagtccaacctgaagcccttcgaaagggatatcagcaccgaaatctaccaggccggctcaaagccttgcaacggggtggaggggttcaactgctacttcccactgcagagttacgggttccagcctaccaacggcgtggggtaccagccctatagagttgtggtgctgagcttcgagctcctgcatgcccccgccaccgtgtgcggacccaagaaatcaaccaacctggtgaagaacaaatgtgtgaacttc

SEQ ID NO: 152 - DNA, 669 bp, unidentified
  source 1..669: mol_type = other DNA
cgggtgcagcctaccgaaagcatcgtgagattcccaaacatcaccaatctgtgccctttcgacgaggtgttcaacgccacaagattcgccagtgtttacgcctggaaccggaagagaatcagcaattgcgtggccgactactctgtgctgtacaaccttgccccttttttcaccttcaagtgttacggcgtgagccccacaaagctgaatgacctgtgtttcaccaacgtgtacgccgactctttcgtgatccggggcgacgaggtcagacagatcgcccctggccagaccggaaacatcgctgattacaactacaagctgcctgatgatttcaccggctgcgttattgcttggaatagcaacaagctggactccaaggtgagcggaaattacaactacctgtacagactgttcagaaagagcaaccttaaacccttcgaaagagacatcagcacagagatctaccaggccggcaacaaaccctgtaacggcgtggccggcttcaactgctacttccccctgagaagttacagctttagacccacatacggagtcggccaccagccctatagagtggtggtgctgagctttgaactgctgcacgccccagctaccgtgtgcggccccaaaaagagcaccaacctggtcaagaacaagtgcgtgaacttc

SEQ ID NO: 153 - DNA, 669 bp, unidentified
  source 1..669: mol_type = other DNA
agagtgcagcccaccgagtcaattgtgaggtttcctaacatcaccaatctgtgtccattcgacgaggtgttcaacgccactaggttcgccagcgtgtacgcctggaaccggaagcgaatttccaattgtgtggcagactacagcgtgctgtacaaccttgctcctttctttacatttaagtgctacggcgtgagccctacaaagctgaatgacctgtgtttcaccaatgtgtacgccgattccttcgtgatcaggggcgacgaggtgagacagatcgcccctggccagacagggaatattgccgactataactataagctcccggatgactttaccggatgtgtgattgcttggaactccaacaaactggacagcaaagtgagcggcaactataactatctgtaccggctgtttcgcaagtctaacctgaaaccttttgagcgggacatctccactgagatctaccaggccgggaataaaccatgcaacggtgtggccggctttaactgctacttcccgctgaggtcttacagcttcaggccaacttatggggtgggccaccagccatatcgcgtggtggtgctgtcctttgagctgctgcacgcccctgccaccgtgtgcggacctaaaaaaagcacaaaccttgtgaaaaacaaatgcgtgaatttc

SEQ ID NO: 154 - DNA, 669 bp, unidentified
  source 1..669: mol_type = other DNA
agagttcagccgactgagtctattgtgcggtttcctaacatcaccaacctgtgccccttcgacgaggtgtttaacgccacaaaattcgccagcgtgtacgcctggaaccggaaaagaatcagcaactgcgtggccgactactctgtactgtacaacctggcccctttcttcaccttcaagtgctacggagtgtcccctaccaagctgaacgatctgtgcttcaccaacgtgtacgccgacagcttcgtgatcagaggagatgaagtgcggcaaatcgcacccggccagacaggcaatattgccgattataactacaagctgcccgacgacttcaccggatgcgtgattgcttggaacagcaacaagctggactccaaggtttctggcaactacaactacctgtacagactgttcagaaagtccaacctgaagcctttcgagagagatatctctaccgagatctaccaagccggaaacaagccctgtaatggcgttgctggctttaactgctacttccccctgagatcttacagcttcagacctacctacggcgtgggccaccagccttaccgggtcgtggtgctgtctttcgaactgcttcacgccccagccacagtgtgcggccctaagaagtctaccaatctggtgaagaacaagtgtgtgaacttc

SEQ ID NO: 155 - DNA, 669 bp, unidentified; identical to SEQ ID NO: 119
  source 1..669: mol_type = other DNA

SEQ ID NO: 156 - DNA, 1392 bp, unidentified
  source 1..1392: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 157 - DNA, 1422 bp, unidentified
  source 1..1422: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 158 - DNA, 1437 bp, unidentified
  source 1..1437: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctggcggtggcggatcggagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 159 - DNA, 1452 bp, unidentified
  source 1..1452: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctggcggtggcggatcgggaggcggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 160 - DNA, 1440 bp, unidentified
  source 1..1440: mol_type = other DNA
atgtttgtttttcttgttttattgccactagtctctaagccaactgagttggagagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 161 - DNA, 1281 bp, unidentified
  source 1..1281: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 162 - DNA, 1311 bp, unidentified
  source 1..1311: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 163 - DNA, 1326 bp, unidentified
  source 1..1326: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctggcggtggcggatcgacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 164 - DNA, 1341 bp, unidentified
  source 1..1341: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctggcggtggcggatcgggaggcggtggctctacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 165 - DNA, 1443 bp, unidentified
  source 1..1443: mol_type = other DNA
atgtttgtttttcttgttttattgccactagtctctaagccaactgagttggagagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcgagaacctgtacttccagggcgagcccaaatcttgtgacaaaactcacacatgcccaccgtgcccagcacctgaagcagaagggggaccgtcagtcttcctcttccccccaaaacccaaggaccagctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctccatgaggctctgcacaaccactacacgcagaagagcctctccctgtccccgggtaaatag

SEQ ID NO: 166 - DNA, 1431 bp, unidentified
  source 1..1431: mol_type = other DNA
atgtttgtttttcttgttttattgccactagtctctaagccaactgagttggagagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcgagaacctgtacttccagggcgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 167 - DNA, 1434 bp, unidentified
  source 1..1434: mol_type = other DNA
atgtttgtttttcttgttttattgccactagtctctggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatggtggaggcggttcaggcggaggtggctctggcggtggcggatcggagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 168 - DNA, 1893 bp, unidentified
  source 1..1893: mol_type = other DNA
atgtttgtttttcttgttttattgccactagtctctacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctggcggtggcggatcggagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 169 - DNA, 1425 bp, unidentified
  source 1..1425: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaaggtggaggcggttcaggcggaggtggctctggcggtggcggatcggagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 170 - DNA, 1329 bp, unidentified
  source 1..1329: mol_type = other DNA
atgaagaccatcatcgccctgagctacatcttctgcctggtgttcgccactaatctttgtccgttcggtgaggtttttaacgcgacaaggttcgctagtgtatatgcttggaaccgaaagagaatctccaattgcgtagctgattactccgttctctataacagtgcgtccttttcaacctttaagtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtggtaattataattaccggtatcgcctgtttcgaaagagcaatttgaagccttttgagcgggatatttcaaccgaaatttaccaagcaggcagtaaaccatgtaacggagtagagggatttaattgctactttcctcttcaatcttatggctttcaaccaacaaacggagtggggtatcaaccttatagagtggtagtattgtcctttgagctcctccacgccccggctacagtttgtgggcccaaaaagggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 171 - DNA, 1326 bp, unidentified
  source 1..1326: mol_type = other DNA
atgaagaccatcatcgccctgagctacatcttctgcctggtgttcgccactaatctttgtccgttcggtgaggtttttaacgcgacaaggttcgctagtgtatatgcttggaaccgaaagagaatctccaattgcgtagctgattactccgttctctataacagtgcgtccttttcaacctttaagtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtggtaattataattaccggtatcgcctgtttcgaaagagcaatttgaagccttttgagcgggatatttcaaccgaaatttaccaagcaggcagtaaaccatgtaacggagtagagggatttaattgctactttcctcttcaatcttatggctttcaaccaacaaacggagtggggtatcaaccttatagagtggtagtattgtcctttgagctcctccacgccccggctacagtttgtgggcccaaaaaggagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctag

SEQ ID NO: 172 - DNA, 1401 bp, unidentified
  source 1..1401: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 173 - DNA, 1323 bp, unidentified
  source 1..1323: mol_type = other DNA
atgaagaccatcatcgccctgagctacatcttctgcctggtgttcgccactaatctttgtccgttcggtgaggtttttaacgcgacaaggttcgctagtgtatatgcttggaaccgaaagagaatctccaattgcgtagctgattactccgttctctataacagtgcgtccttttcaacctttaagtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtggtaattataattaccggtatcgcctgtttcgaaagagcaatttgaagccttttgagcgggatatttcaaccgaaatttaccaagcaggcagtaaaccatgtaacggagtagagggatttaattgctactttcctcttcaatcttatggctttcaaccaacaaacggagtggggtatcaaccttatagagtggtagtattgtcctttgagctcctccacgccccggctacagtttgtgggcccaaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 174 - DNA, 1332 bp, unidentified
  source 1..1332: mol_type = other DNA
atgaagaccatcatcgccctgagctacatcttctgcctggtgttcgccactaatctttgtccgttcggtgaggtttttaacgcgacaaggttcgctagtgtatatgcttggaaccgaaagagaatctccaattgcgtagctgattactccgttctctataacagtgcgtccttttcaacctttaagtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtggtaattataattaccggtatcgcctgtttcgaaagagcaatttgaagccttttgagcgggatatttcaaccgaaatttaccaagcaggcagtaaaccatgtaacggagtagagggatttaattgctactttcctcttcaatcttatggctttcaaccaacaaacggagtggggtatcaaccttatagagtggtagtattgtcctttgagctcctccacgccccggctacagtttgtgggcccaaaaagggatccgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggctga

SEQ ID NO: 175 - DNA, 1392 bp, unidentified
  source 1..1392: mol_type = other DNA
atgtttgtttttcttgttttattgccactagtctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 176 - DNA, 1422 bp, unidentified
  source 1..1422: mol_type = other DNA
atgtttgtttttcttgttttattgccactagtctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 177 - DNA, 1437 bp, unidentified
  source 1..1437: mol_type = other DNA
atgtttgtttttcttgttttattgccactagtctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 178 - DNA, 1452 bp, unidentified
  source 1..1452: mol_type = other DNA
atgtttgtttttcttgttttattgccactagtctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 179 - DNA, 2151 bp, unidentified
  source 1..2151: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 180 - DNA, 2151 bp, unidentified
  source 1..2151: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 181 - DNA, 2040 bp, unidentified
  source 1..2040: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 182 - DNA, 2040 bp, unidentified
  source 1..2040: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 183 - DNA, 2151 bp, unidentified
  source 1..2151: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 184 - DNA, 2181 bp, unidentified
  source 1..2181: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggaggcggtggctctggcggtggcggatcgggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 185 - DNA, 2121 bp, unidentified
  source 1..2121: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 186 - DNA, 2151 bp, unidentified
  source 1..2151: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctggcggtggcggatcgggaggcggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggtggcggatcgagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctag

SEQ ID NO: 187 - DNA, 2151 bp, unidentified
  source 1..2151: mol_type = other DNA
atgtttgtttttcttgttttattgccactagtctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttctag

SEQ ID NO: 188 - DNA, 2181 bp, unidentified
  source 1..2181: mol_type = other DNA
atgttcgtcttcctggtgctgctgccactggtcagcagagtgcagcctaccgaatctatcgtgcggttccctaacatcaccaatctgtgcccctttggagaggtgttcaatgcgactcggttcgcttctgtgtacgcttggaacagaaagcggatcagcaactgcgtcgccgactacagcgtgctgtacaacagcgccagctttagcacctttaagtgctacggcgtgtctcctaccaagctgaatgatctgtgtttcaccaacgtgtacgccgacagctttgtcatccgtggagatgaggtgagacagatcgcccctggccagaccggcaaaatcgccgattacaactacaagctgcccgatgacttcaccggatgtgtgatcgcctggaactctaacaacctggactccaaggtgggcgggaattataactacagatacagactgttcagaaagagcaatctcaagcctttcgagagagatatttccacagaaatctaccaggccggcagcaaaccttgtaacggcgttgaaggctttaattgctacttccctctgcagagctacggcttccaacctacaaacggcgtgggctatcagccgtacagagtcgtggtcctgtcctttgagctgcttcatgcccctgctaccgtgtgcggtcctaaaaagagtacaaacctggtgaagaacaagtgtgtgaacttcggcggaggaggaagcggcggcggcggcagcggcggcggagggtctggaggcggcggcagcgagtccaaatacggccctccatgccccccctgtcctgcccctgaagccgctggcggcccaagcgtgttcctgtttcccccaaagcccaaggacaccctgatgattagccggacccctgaggtgacctgtgtggtggtggacgtgtcccaggaggaccccgaagtgcagttcaactggtacgtcgacggagtggaagtgcacaacgccaagaccaagcctagagaagagcagttcaattcaacatacagagtggtgagcgtgctgaccgtgctgcaccaggactggctgaacggcaaggaatacaagtgcaaggtgagcaacaagggcctgcctagcagcatcgagaagaccatcagcaaggccaaaggccagcctagagagcctcaggtgtacaccctgcctcccagccaggaggaaatgaccaaaaaccaggtgtccctgacatgcctggtcaagggcttctacccttctgatatcgccgtggaatgggagagcaacggccagcctgagaacaactacaaaacaacccctcctgtgctcgatagcgacggcagtttcttcctctactctcggctgacagtggataagagcagatggcaggagggcaacgtgttttcttgtagcgtgatgcacgaggccctgcacaaccactacacccagaagtctctgtctctgagcctgggcggcggaggaggtagcggcggaggcggcagcggcggcggcggaagcggcggcggaggctccagagtgcagcccacagaaagcatcgtgcgcttccccaacatcaccaacctgtgcccattcggcgaggtattcaacgccacccggtttgcctctgtgtatgcctggaatagaaagcggatcagcaactgcgtggccgattacagcgtgctgtacaacagcgcttctttcagcaccttcaagtgctacggcgtgagccctaccaagctgaacgacctgtgcttcacaaatgtgtacgccgacagcttcgtgatcagaggcgacgaggtcagacagatcgcccctggccaaacaggaaacatcgccgactataattacaagctgcccgacgacttcaccggctgcgtgattgcttggaatagcaacaacctggacagcaaggtgggaggcaactacaactatctgtatcggctgttcagaaagagtaatctgaagcctttcgagcgggacatcagcaccgagatctaccaagccggcagcacaccctgcaacggcgtgaagggcttcaactgctacttccccctgcaaagctacggcttccagcccacctacggcgtgggctaccagccataccgggtggttgttctgtcgttcgaactactgcacgcccctgccacagtgtgcggccccaagaaatctacaaacctggtgaagaacaaatgcgtaaacttctga

SEQ ID NO: 189 - DNA, 2169 bp, unidentified
  source 1..2169: mol_type = other DNA
atgttcgtgttcctggtgctgctacccctggtgtctagagtccagcctacagaatctatcgtgagattccctaacatcaccaacctgtgccctttcggcgaggtctttaacgccacaagattcgcctccgtgtacgcctggaatagaaagcggatcagcaactgcgttgctgattatagcgtcctgtacaactctgctagcttcagcaccttcaagtgctacggcgtgagccccacaaagctgaatgacctgtgttttaccaacgtgtatgctgactctttcgtaatccggggcgacgaggttcggcagatcgctccaggccagaccggcaagatcgccgattacaactacaagctgcccgacgacttcacaggatgtgtgattgcctggaacagcaacaacctggacagcaaggtgggcggcaattacaactacagataccggctgtttagaaagtccaacctgaagcctttcgagagagacatcagcaccgaaatctaccaagctggcagcaaaccttgtaatggagtggaaggcttcaactgctatttccctctgcaatcttatggcttccagcccaccaacggcgtgggataccagccttaccgcgtggtggtgctgagcttcgagctgctgcatgccccagccacagtgtgtggccctaagaagtccactaacctggtgaagaacaaaggcgggggcggcagcggaggaggtggctccggaggcggcggcagcggcggaggcggctctgagagcaagtacgggcctccttgtcctccttgccccgcccctgaagccgccggcggaccttctgtgttcctgttcccccctaagcccaaggacaccctgatgatctctagaacacccgaggtgacctgcgtggtggtggacgtgtcccaggaggaccccgaggttcagtttaactggtatgtggacggcgtggaggtgcacaacgctaaaaccaagcccagagaagagcagttcaacagcacctaccgggtggtgagcgtgctgaccgtgctgcaccaggactggctgaacggcaaggaatacaaatgtaaagtgagcaacaagggcctgccttcttccatcgagaagaccatcagcaaggccaagggccagcctagagagcctcaggtgtacaccctgcccccaagccaggaggaaatgaccaaaaaccaagtgtccctgacctgtctggtgaagggattctaccctagcgacatcgccgtggaatgggaaagcaacggccagcccgaaaacaactacaaaaccacccctcctgtgttggattctgatggcagcttcttcctgtacagcagactcaccgtggacaagtctagatggcaggagggcaacgtgttctcatgctccgtgatgcacgaggccctgcacaatcactacacacagaagagcctgagcctgagcctgggcggcggcggtggcagcggtggcggcggcagcggcggtggaggtagcggcggaggaggcagcagagtgcagcccaccgaaagcatcgtgcgcttccctaatatcaccaatttgtgcccatttggcgaggtgttcaacgccacacggtttgcctccgtgtacgcctggaaccggaagagaatctctaattgcgtggccgactacagcgttctgtacaacagcgcctctttctctacttttaagtgctacggcgtctctcctacaaagctgaacgacctgtgcttcaccaacgtgtacgccgacagctttgtgatcaggggcgatgaggtccggcagatcgcccctggccaaacaggaaacattgccgactacaattacaagctgccagatgatttcaccggctgtgtgatcgcctggaattcgaacaacctggatagcaaagtgggcggcaactacaattacctgtacagactgttcagaaagagcaatctgaagcccttcgaaagagatatcagcacagagatctaccaggccggaagcacaccttgcaacggcgtgaaaggattcaactgctacttcccactgcagagctacggcttccagcctacctacggcgtcggctaccagccttaccgagtggtcgtgctgagctttgagctgctgcacgcccctgctacagtgtgcggccccaagaaaagcaccaacctggtcaagaacaagtgcgtgaacttctga

SEQ ID NO: 190 - DNA, 2169 bp, unidentified
  source 1..2169: mol_type = other DNA
atgttcgtgttcctggtgctgctgccactggtgagccgggtccagcctaccgagagtatcgtgagattccctaatatcaccaacctgtgtcctttcggcgaagtgttcaacgccacaagattcgccagcgtgtacgcctggaatagaaagagaatctccaattgcgtggccgactacagcgtgctgtacaactctgccagcttttctacatttaagtgctacggcgtgtctcctaccaagctgaatgatctgtgctttacaaacgtgtacgccgacagcttcgtgatcagaggcgacgaggtgcgccagatcgcccctggacaaactggcaagatcgccgattacaactacaagctgcctgatgacttcaccggctgcgtgatcgcatggaacagcaacaacctggacagcaaggtgggcggcaactacaactaccggtacagactgtttcggaagagcaacctgaaacctttcgagagagatatctccaccgagatctaccaggccggatccaagccgtgcaatggcgtggaaggcttcaactgctacttcccactgcaatcttatggcttccagccaaccaacggcgttggatatcagccctatcgggtagtggtgttgtcttttgagctgctgcacgcccctgccacagtgtgtggacccaagaagagcaccaacctggtcaagaacaaaggcggcggcggctccggcggcggcggtagcggaggtggcgggagtggcggcggcggctctgagagcaaatacggcccaccatgtcccccctgtcctgctcccgaggccgccggcggccccagcgttttcctgttcccccctaagcccaaggacaccctgatgatctcccggacccctgaagtcacatgcgtggtggtggacgtgtcccaggaggaccccgaggtgcagttcaattggtacgtggacggagtggaagtgcacaacgctaagacaaaacctagggaagaacagttcaacagcacatacagagtggttagcgtactcacagtgctgcaccaggactggctgaatggaaaggaatacaagtgcaaggtgtccaacaaggggctgccttcaagcatcgagaagaccatctctaaagccaagggccagcccagagagcctcaggtgtacaccctgcctcctagccaggaggaaatgaccaaaaaccaggttagcctgacctgcctggtgaaaggcttctacccttctgatatcgccgtggaatgggagagcaacggacaacctgagaacaactacaagaccacccctcctgtgctggacagcgacggctctttcttcctctactccagactcaccgtggacaagtctagatggcaggagggcaatgtgttctcatgcagcgtcatgcacgaggccctgcataatcactacacacagaagagcctgagcctgagcctcggaggcggcggaggaagcggcggcggcggcagcggaggcggcggcagcggaggcggcggctctagagtgcagcccaccgaaagcattgtgcggttccccaacatcacaaacctgtgccctttcggcgaggtgttcaacgccacccgctttgctagcgtttacgcctggaacagaaagagaatcagcaattgcgtggccgattacagcgtgctgtacaacagcgcctctttcagcacattcaagtgttatggcgtcagccctacaaagctgaacgacctgtgcttcaccaacgtgtacgccgacagctttgtgattagaggcgatgaggtgcgacagatcgcccctggccagaccggcaagatcgccgactacaattataagctgcctgacgatttcaccggctgtgtgatcgcctggaactctaacaacctggatagcaaggtgggaggaaactacaactaccagtacagactgttccggaagtccaacctgaagccctttgagcgggacatcagcaccgagatctaccaagctggctcaacaccttgcaacggcgttgaaggctttaactgttacagccctctgcagtcctacggtttccagcctaccaacggcgtgggctaccagccctaccgggtggtggtcctgagcttcgagctgctgcacgccccggctaccgtgtgcggccccaagaaaagcaccaacctggtgaaaaacaagtgcgtgaacttctga

SEQ ID NO: 191 - DNA, 2124 bp, unidentified
  source 1..2124: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggcggtggcggatcgggaggcggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 192 - DNA, 2061 bp, unidentified
  source 1..2061: mol_type = other DNA
atgtttgtttttcttgttttattgccactagtctctacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagggaggaggcggatccggaggaggaggatccggcggaggcggaagcgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 193 - DNA, 2022 bp, unidentified
  source 1..2022: mol_type = other DNA
atgtttgtttttcttgttttattgccactagtctctacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagggatccgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 194 - DNA, 2139 bp, unidentified
  source 1..2139: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggaggaggcggatccggaggaggaggatccggcggaggcggaagcgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 195 - DNA, 2100 bp, unidentified
  source 1..2100: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggatccgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 196 - DNA, 2124 bp, unidentified
  source 1..2124: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggcggtggcggatcgggaggcggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 197 - DNA, 2124 bp, unidentified
  source 1..2124: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggcggtggcggatcgggaggcggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgatgcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 198 - DNA, 2124 bp, unidentified; identical to SEQ ID NO: 196
  source 1..2124: mol_type = other DNA

SEQ ID NO: 199 - DNA, 2124 bp, unidentified; identical to SEQ ID NO: 197
  source 1..2124: mol_type = other DNA

SEQ ID NO: 200 - DNA, 2139 bp, unidentified
  source 1..2139: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggaggaggcggatccggaggaggaggatccggcggaggcggaagcgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 201 - DNA, 2139 bp, unidentified
  source 1..2139: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggaggaggcggatccggaggaggaggatccggcggaggcggaagcgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgatgcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 202 - DNA, 2169 bp, unidentified
  source 1..2169: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggaggaggcggatccggaggaggaggatccggcggaggcggaagcgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 203 - DNA, 2154 bp, unidentified
  source 1..2154: mol_type = other DNA
atgtttgtttttcttgttttattgccactagtctctagagtccagccaactgaatctattgtgcgcttcccgaacatcacaaacttgtgcccatttggggaagtgtttaacgctaccaaattcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacctgtataggcttttccgcaaatcaaacctgaagccctttgaaagggacattagcaccgagatttatcaagccggcagcacaccgtgtaatggtgttaagggcttcaactgttactttcccctccagtcttatggatttcagcctacttacggtgtaggctatcagccatacagagtagtagtccttagttttgagttgctgcatgcaccggcaacagtttgtggccctaagaagtcaacaaatctggtgaaaaataagtgcgtcaatttcggcggtggcggatcgggaggcggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 204 - DNA, 2154 bp, unidentified
  source 1..2154: mol_type = other DNA
atgtttgtttttcttgttttattgccactagtctctagagtccagccaactgaatctattgtgcgcttcccgaacatcacaaacttgtgcccatttgatgaagtgtttaacgctacccggttcgcttccgtgtatgcttggaacagaaaacgaataagtaactgcgttgcggattactctgtgttgtataatcttgcgcccttctttacattcaagtgctacggagtatcccctacgaagcttaacgatttgtgttttacgaacgtctatgcggattcctttgtcattcgcggggacgaggtgagacagattgcacctggacaaacggggaacattgcggattataactataaacttcccgatgatttcacagggtgcgtgatagcgtggaactcaaataagcttgatagtaaggtatcaggaaattacaattacctttataggcttttccgcaaatcaaacctgaagccctttgaaagggacattagcaccgagatttatcaagccggcaataagccgtgtaatggtgttgcgggcttcaactgttactttcccctcagatcttattcctttagacctacttatggtgtaggccatcagccatacagagtagtagtccttagttttgagttgctgcatgcaccggcaacagtttgtggccctaagaagtcaacaaatctggtgaaaaataagtgcgtcaatttcggcggtggcggatcgggaggcggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 205 - DNA, 2154 bp, unidentified
  source 1..2154: mol_type = other DNA
atgttcgtgttcttagtgctgctgcctctggtgagcagagtgcagcccaccgagtccatcgtgaggttccctaacattaccaatctgtgtcccttcgatgaagtgttcaatgccacccgcttcgcttctgtctacgcctggaaccgcaagaggattagcaactgcgtggccgactactccgtgctgtataacctggcccctttcttcaccttcaagtgctacggcgtgtcccccactaagctgaatgacctgtgcttcacaaacgtgtatgctgacagtttcgtcatcagaggcgacgaagtgaggcagatagcccctgggcagactgggaacatcgccgactataactacaagctgccagatgatttcacaggctgcgtgatcgcttggaacagcaataagctggatagcaaggtctctggcaactataactatctgtaccgcctgtttcggaaatctaatctgaagcccttcgagcgggacatcagcaccgaaatctaccaggctggcaataagccatgcaacggcgtggccggcttcaattgttattttcctctgagatcttacagtttcaggcctacctacggggtgggccaccagccctatcgggtggtggtgctgagcttcgagctgctgcatgcccccgccactgtgtgtgggccaaagaaatccacaaacctggtgaagaacaaatgtgtgaactttggaggaggcggctccgggggcggtggcagcgagtccaaatacggcccaccttgccctccctgccctgctcccgaggctgccggaggcccctccgtgttcctgttcccacccaagcccaaggacaccctgatgatctctaggacccccgaagtgacttgcgtggtggtcgacgtgagccaggaggatccagaagtgcagttcaactggtacgtggacggcgtggaggtgcacaatgcaaagacaaaacccagggaggagcagttcaactccacataccgggtggtcagcgtgctgaccgtgctggcacaggactggctgaatggcaaggagtataagtgcaaagtgtccaacaaggggctgccaagctccatcgagaagaccatctccaaggccaagggtcagcccagggagcctcaggtgtataccctgcccccctctcaggaagagatgactaagaaccaggtgtccctgacatgcctggtgaaaggattttaccccagcgacatcgccgtggagtgggagagcaatggccagcccgaaaacaactacaagacaacccctcccgtgctggacagcgatggatcattcttcctgtacagcaggctgacagtggacaaatcacggtggcaggagggaaacgtgttcagctgctccgtgatgcacgaggccctccacaatcactacacacagaaaagcctgagcctgtcactgggcggtggcggcggatccggaggcggcggatctggcggcggcggcagcggcggaggcggatcaagggttcagccaactgaatccatcgtgcgcttccccaacattaccaatctgtgtccattcggggaggtgttcaacgccactaggttcgccagcgtgtacgcctggaaccggaagcgcatcagcaactgcgtggccgactattccgtgctgtataactctgcctccttttccacttttaagtgctacggggtgtctccaacaaagctgaatgacctgtgctttaccaatgtgtacgccgatagcttcgtgatcaggggcgatgaagtccggcagatcgcccccgggcagacaggcaagatcgccgattataattataagctgccagacgatttcaccggctgcgtgatcgcatggaatagcaacaatctggactccaaagtgggggggaattataactaccgctaccgcctgttccgcaaatccaatctgaagccattcgagagagacatttccacagagatttatcaggccggctctaaaccctgcaacggcgtggaaggattcaattgttacttccccctgcagtcatatggcttccagcctaccaacggcgtgggctatcagccttatcgggtggtggtgctgtctttcgagctgctgcatgcacccgccacagtgtgtggcccaaagaaaagcaccaatctggtgaaaaacaagtgcgtgaacttctgatga

SEQ ID NO: 206 - DNA, 2139 bp, unidentified
  source 1..2139: mol_type = other DNA
atgtttgtttttcttgttttattgccactagtctctagagtccagccaactgaatctattgtgcgcttcccgaacatcacaaacttgtgcccatttgatgaagtgtttaacgctacccggttcgcttccgtgtatgcttggaacagaaaacgaataagtaactgcgttgcggattactctgtgttgtataatcttgcgcccttctttacattcaagtgctacggagtatcccctacgaagcttaacgatttgtgttttacgaacgtctatgcggattcctttgtcattcgcggggacgaggtgagacagattgcacctggacaaacggggaacattgcggattataactataaacttcccgatgatttcacagggtgcgtgatagcgtggaactcaaataagcttgatagtaaggtatcaggaaattacaattacctttataggcttttccgcaaatcaaacctgaagccctttgaaagggacattagcaccgagatttatcaagccggcaataagccgtgtaatggtgttgcgggcttcaactgttactttcccctcagatcttattcctttagacctacttatggtgtaggccatcagccatacagagtagtagtccttagttttgagttgctgcatgcaccggcaacagtttgtggccctaagaagtcaacaaatctggtgaaaaataagtgcgtcaatttcggaggaggcggatccggaggaggaggatccggcggaggcggaagcgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagactgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 207 - DNA, 2139 bp, unidentified
  source 1..2139: mol_type = other DNA
atgttcgtgttcctggtgctgctgcccctggtcagtagagttcagccgactgagtctattgtgcggtttcctaacatcaccaacctgtgccccttcgacgaggtgtttaacgccacaagattcgccagcgtgtacgcctggaaccggaaaagaatcagcaactgcgtggccgactactctgtactgtacaacctggcccctttcttcaccttcaagtgctacggagtgtcccctaccaagctgaacgatctgtgcttcaccaacgtgtacgccgacagcttcgtgatcagaggagatgaagtgcggcaaatcgcacccggccagacaggcaatattgccgattataactacaagctgcccgacgacttcaccggatgcgtgattgcttggaacagcaacaagctggactccaaggtttctggcaactacaactacctgtacagactgttcagaaagtccaacctgaagcctttcgagagagatatctctaccgagatctaccaagccggaaacaagccctgtaatggcgttgctggctttaactgctacttccccctgagatcttacagcttcagacctacctacggcgtgggccaccagccttaccgggtcgtggtgctgtctttcgaactgcttcacgccccagccacagtgtgcggccctaagaagtctaccaatctggtgaagaacaagtgtgtgaacttcggcggcggaggatccggcggcggcggcagcggaggcggtgggagcgagagcaagtacggccctccttgccctccttgtcccgctccagaagccgctggaggccctagcgtgttcctgtttccacccaaaccaaaggacaccctgatgatcagcagaacacctgaagtgacctgtgtggtggtggacgtgagccaggaggaccccgaggttcaatttaattggtacgtggatggcgtggaagtgcacaatgccaaaacaaagcccagagaggaacagttcaacagcacataccgagtggtgagcgtgctgacagtgcttgcccaggactggctgaacggaaaagagtacaagtgtaaagtgagcaataaaggcctgcccagcagcatcgagaagacaatcagcaaggccaagggccagcctagagaaccccaggtgtacaccctgcctccatctcaggaggaaatgacaaagaaccaggtgtccctgacctgcctggtgaagggcttctacccctctgacatcgccgtggaatgggagagcaacggccagcctgagaacaactacaagaccacaccccctgtgctggacagcgacggcagctttttcctgtacagccgcctgaccgtggacaagtccagatggcaggagggcaacgtgttctcatgcagtgtgatgcacgaggccctgcacaaccactacacccagaaatctctgtccctgagcctgggcggcggcggaggcagcggcggcggaggcagcagagtgcagcctaccgagagcatcgtgcggttccccaatatcaccaacctgtgcccttttggcgaggtgtttaacgccaccagattcgcctctgtgtatgcttggaatagaaagagaatcagcaactgcgtcgctgattactccgtcctctataattctgccagcttcagcaccttcaagtgctacggtgtcagccctaccaagctgaatgacctgtgttttaccaatgtctacgccgatagcttcgtgatccggggcgacgaggtgcggcagatcgcccctggccaaacaggcaagatcgccgattacaactacaagctgcctgatgacttcacaggctgcgtgatcgcttggaacagcaacaacctggatagcaaggtgggaggcaactacaactaccggtacagactcttccggaagtctaacctgaagcctttcgaacgggacatcagcaccgaaatctaccaggccggcagcaagccttgcaacggcgtggaaggcttcaattgctatttccctctgcagagctatggcttccagccaaccaacggcgtgggctaccagccttacagggtggtggtgttgtctttcgagctgctgcatgcccctgccaccgtttgtggcccaaagaaaagcaccaacctggttaagaacaagtgcgtgaacttctgatga

SEQ ID NO: 208 - DNA, 2139 bp, unidentified
  source 1..2139: mol_type = other DNA
atgttcgtgttcctggtgctgctccctctggtgagccgggtgcagcctaccgagtctatcgtgaggttccctaatatcaccaacctgtgtcctttcgacgaagtcttcaacgccaccagatttgccagcgtgtatgcctggaacaggaagagaatctctaactgcgtggccgactactctgtgctatacaacctggccccctttttcacattcaagtgctacggcgtgtcccctaccaaactgaacgacctgtgttttaccaatgtgtacgccgatagcttcgtgatcaggggcgacgaggtgcgccagattgcccctggacagactggaaacatcgccgactataattacaagctgccagacgacttcaccggctgcgtgattgcatggaattctaataaactggattccaaggtttccggcaattacaactacctgtaccgcctgttccggaagtccaacctgaagcccttcgaaagggatatcagcaccgaaatctaccaggccggcaacaagccttgcaacggggtggctgggttcaactgctacttcccactgagaagttacagcttccgccctacctacggcgtggggcaccagccctatagagttgtggtgctgagcttcgagctcctgcatgcccccgccaccgtgtgcggacccaagaaatcaaccaacctggtgaagaacaaatgtgtgaacttcggggggggcggatctggaggcggcggctccggcggcggcggcagcgaatccaagtacggacccccttgtccaccctgccctgcccccgaggccgctggcggcccctccgtgtttctgttcccccccaagcccaaggacaccctgatgatcagccgcaccccagaggtgacttgcgtggtggtggatgtgtcccaggaggacccagaggtgcagttcaactggtatgtggacggggtggaggtgcacaacgccaagaccaagcctagggaggagcagttcaatagcacttatagggtggtgtccgtgttgacagtgctggcccaggactggctgaatggcaaggagtataagtgcaaagtcagcaataagggactccctagctccatcgaaaaaaccatctccaaggctaaggggcagcccagggagccccaggtgtacaccctgccccccagccaggaggagatgaccaagaaccaggtgagtctcacttgcctggtgaagggcttttacccttccgacattgcagtggagtgggaaagcaacggacagccagaaaataactataagactacccctcccgtgctggactccgatgggtcattctttctgtacagcaggctgacagtggataagagcagatggcaggagggcaacgtcttttcctgtagcgtgatgcacgaagccctgcacaatcactacacccagaagtccctgagcctgagcctgggtggcggcggcgggagcgggggaggcggctccagagtgcagcccaccgagtcaattgtgaggtttcctaacatcaccaatctgtgtccattcggcgaggtgttcaacgccactaggttcgccagcgtgtacgcctggaaccggaagcgaatttccaattgtgtggcagactacagcgtgctgtacaactccgctagcttcagcacatttaagtgctacggcgtgagccctacaaagctgaatgacctgtgtttcaccaatgtgtacgccgattccttcgtgatcaggggcgacgaggtgagacagatcgcccctggccagacagggaagattgccgactataactataagctcccggatgactttaccggatgtgtgattgcttggaactccaacaacctggacagcaaagtgggtggcaactataactatagataccggctgtttcgcaagtctaacctgaaaccttttgagcgggacatctccactgagatctaccaggccgggagcaaaccatgcaacggtgtggaaggctttaactgctacttcccgctgcagtcttacggattccagccaactaatggggtgggctaccagccatatcgcgtggtggtgctgtcctttgagctgctgcacgcccctgccaccgtgtgcggacctaaaaaaagcacaaaccttgtgaaaaacaaatgcgtgaatttctgatga

SEQ ID NO: 209 - DNA, 2154 bp, unidentified
  source 1..2154: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctagtga

SEQ ID NO: 210 - DNA, 2157 bp, unidentified
  source 1..2157: mol_type = other DNA
atgcacagctcagcactgctctgttgcctggtcctcctgactggggtgagggccagagttcagccgactgagtctattgtgcggtttcctaacatcaccaacctgtgccccttcgacgaggtgtttaacgccacaagattcgccagcgtgtacgcctggaaccggaaaagaatcagcaactgcgtggccgactactctgtactgtacaacctggcccctttcttcaccttcaagtgctacggagtgtcccctaccaagctgaacgatctgtgcttcaccaacgtgtacgccgacagcttcgtgatcagaggagatgaagtgcggcaaatcgcacccggccagacaggcaatattgccgattataactacaagctgcccgacgacttcaccggatgcgtgattgcttggaacagcaacaagctggactccaaggtttctggcaactacaactacctgtacagactgttcagaaagtccaacctgaagcctttcgagagagatatctctaccgagatctaccaagccggaaacaagccctgtaatggcgttgctggctttaactgctacttccccctgagatcttacagcttcagacctacctacggcgtgggccaccagccttaccgggtcgtggtgctgtctttcgaactgcttcacgccccagccacagtgtgcggccctaagaagtctaccaatctggtgaagaacaagtgtgtgaacttcggcggcggaggatccggcggcggcggcagcggaggcggtgggagcgagagcaagtacggccctccttgccctccttgtcccgctccagaagccgctggaggccctagcgtgttcctgtttccacccaaaccaaaggacaccctgatgatcagcagaacacctgaagtgacctgtgtggtggtggacgtgagccaggaggaccccgaggttcaatttaattggtacgtggatggcgtggaagtgcacaatgccaaaacaaagcccagagaggaacagttcaacagcacataccgagtggtgagcgtgctgacagtgcttgcccaggactggctgaacggaaaagagtacaagtgtaaagtgagcaataaaggcctgcccagcagcatcgagaagacaatcagcaaggccaagggccagcctagagaaccccaggtgtacaccctgcctccatctcaggaggaaatgacaaagaaccaggtgtccctgacctgcctggtgaagggcttctacccctctgacatcgccgtggaatgggagagcaacggccagcctgagaacaactacaagaccacaccccctgtgctggacagcgacggcagctttttcctgtacagccgcctgaccgtggacaagtccagatggcaggagggcaacgtgttctcatgcagtgtgatgcacgaggccctgcacaaccactacacccagaaatctctgtccctgagcctgggcggcggcggaggcagcggcggcggaggcagcagagtgcagcctaccgagagcatcgtgcggttccccaatatcaccaacctgtgcccttttggcgaggtgtttaacgccaccagattcgcctctgtgtatgcttggaatagaaagagaatcagcaactgcgtcgctgattactccgtcctctataattctgccagcttcagcaccttcaagtgctacggtgtcagccctaccaagctgaatgacctgtgttttaccaatgtctacgccgatagcttcgtgatccggggcgacgaggtgcggcagatcgcccctggccaaacaggcaagatcgccgattacaactacaagctgcctgatgacttcacaggctgcgtgatcgcttggaacagcaacaacctggatagcaaggtgggaggcaactacaactaccggtacagactcttccggaagtctaacctgaagcctttcgaacgggacatcagcaccgaaatctaccaggccggcagcaagccttgcaacggcgtggaaggcttcaattgctatttccctctgcagagctatggcttccagccaaccaacggcgtgggctaccagccttacagggtggtggtgttgtctttcgagctgctgcatgcccctgccaccgtttgtggcccaaagaaaagcaccaacctggttaagaacaagtgcgtgaacttctgatga

SEQ ID NO: 211 - DNA, 2154 bp, unidentified
  source 1..2154: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtccaccaacctggtgaagaacaaatgcgtgaacttctagtga

SEQ ID NO: 212 - DNA, 2172 bp, unidentified
  source 1..2172: mol_type = other DNA
atgcacagctcagcactgctctgttgcctggtcctcctgactggggtgagggccagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtccaccaacctggtgaagaacaaatgcgtgaacttctagtga

SEQ ID NO: 213 - DNA, 2142 bp, unidentified
  source 1..2142: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtccaccaacctggtgaagaacaaatagtga

SEQ ID NO: 214 - DNA, 2205 bp, unidentified
  source 1..2205: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtccaccaacctggtgaagaacaaatgcgtgaacttcaacttcaatggtttaaaaggcacaggtgttcttactgagtctaacaaaaagtagtga

SEQ ID NO: 215 - DNA, 2118 bp, unidentified
  source 1..2118: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtagtga

SEQ ID NO: 216 - DNA, 2085 bp, unidentified
  source 1..2085: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtagtga

SEQ ID NO: 217 - DNA, 2061 bp, unidentified
  source 1..2061: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctagggtgcagccaacagagagcatcgtgaggttcccaaacatcacaaacctgtgcccattcgacgaggtgttcaacgcaaccaggttcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtgtagtga

SEQ ID NO: 218 - DNA, 2085 bp, unidentified
  source 1..2085: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctccaaacatcacaaacctgtgcccattcgacgaggtgttcaacgcaaccaaattcgcaagcgtgtacgcatggaacaggaagaggatcagcaactgcgtggcagactactctgtgctgtataatctggcccccttttttacctttaagtgctatggcgtgagtcccacaaagctgaacgatctgtgcttcaccaacgtgtacgccgactcctttgtgattaggggcgacgaggtgcggcagatcgcccctggacagaccggaaacatcgccgactataactacaagctgcccgacgacttcaccggctgcgtgatcgcctggaactccaacaagctggactccaaagtgtctggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctactccttccgccccacctacggcgtgggacaccagccttaccgggtggtggtgctgtccttcgaactgctgcacgcacccgccaccgtgtgcggacctaaaaagtgatga

SEQ ID NO: 219 - DNA, 2085 bp, unidentified
  source 1..2085: mol_type = other DNA
atgtttgtttttcttgttttattgccactagtctctagagtccagccaactgaatctattgtgcgcttcccgaacatcacaaacttgtgcccatttggggaagtgtttaacgctacccggttcgcttccgtgtatgcttggaacagaaaacgaataagtaactgcgttgcggattactctgtgttgtataattcagcgagcttcagcacattcaagtgctacggagtatcccctacgaagcttaacgatttgtgttttacgaacgtctatgcggattcctttgtcattcgcggggacgaggtgagacagattgcacctggacaaacggggaagattgcggattataactataaacttcccgatgatttcacagggtgcgtgatagcgtggaactcaaataatcttgatagtaaggtaggcggaaattacaattacaggtataggcttttccgcaaatcaaacctgaagccctttgaaagggacattagcaccgagatttatcaagccggcagcaagccgtgtaatggtgttgagggcttcaactgttactttcccctccagtcttatggatttcagcctactaatggtgtaggctatcagccatacagagtagtagtccttagttttgagttgctgcatgcaccggcaacagtttgtggccctaagaagtcaacaaatctggtgaaaaataagtgcgtcaatttcggtggaggcggttcaggcggaggtggctctgagagcaagtacggccccccttgcccaccctgtccagctcctgaggctgctggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctggctcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcggtggaggcggttcaggcggaggtggctctggcggtggcggatcgggaggcggtggctctccaaacatcacaaacctgtgcccattcgacgaggtgttcaacgcaaccaggttcgcaagcgtgtacgcatggaacaggaagaggatcagcaactgcgtggcagactactctgtgctgtataatttcgcccccttttttgcttttaagtgctatggcgtgagtcccacaaagctgaacgatctgtgcttcaccaacgtgtacgccgactcctttgtgattaggggcaatgaggtgagccagatcgcccctggacagaccggaaacatcgccgactataactacaagctgcccgacgacttcaccggctgcgtgatcgcctggaactccaacaagctggactccaaagtgggtggcaactacaattacctgtaccgcctgttccgcaagtccaacctgaagcccttcgaaagggacatctccacagagatctaccaggccggcaacaaaccctgcaacggcgtggctggctttaactgctacttccccctgcgctcctacggtttccgccccacctacggcgtgggacaccagccttaccgggtggtggtgctgtccttcgaactgctgcacgcacccgccaccgtgtgcggacctaaaaagtgatga

SEQ ID NO: 220 - protein, 12 aa, synthetic construct
MFVFLVLLPLVS

SEQ ID NO: 221 - protein, 16 aa, synthetic construct
MKTIIALSYIFCLVFA

SEQ ID NO: 222 - protein, 18 aa, synthetic construct
MHSSALLCCLVLLTGVRA

SEQ ID NO: 223 - protein, 16 aa, synthetic construct
MFVFLVLLPLVSSQCV

SEQ ID NO: 224 - protein, 6 aa, synthetic construct
KPTELE

SEQ ID NO: 225 - skipped

SEQ ID NO: 226 - protein, 7 aa, synthetic construct
ENLYFQG

SEQ ID NO: 227 - protein, 10 aa, synthetic construct
GGGGSGGGGS

SEQ ID NO: 228 - protein, 15 aa, synthetic construct
GGGGSGGGGSGGGGS

SEQ ID NO: 229 - protein, 20 aa, synthetic construct
GGGGSGGGGSGGGGSGGGGS

SEQ ID NO: 230 - protein, 19 aa, synthetic construct
GGGSGGGGSGGGGSGGGGS

SEQ ID NO: 231 - protein, 37 aa, synthetic construct
ESKYGPPCPPCPAPEAAGGPSVFLFPPKPKDTLMISR

SEQ ID NO: 232 - protein, 37 aa, synthetic construct
ESKYGPPCPPCPAPEFEGGPSVFLFPPKPKDTLMISR

SEQ ID NO: 233 - protein, 35 aa, synthetic construct
DKTHTCPPCPAPELLGGPSVFLFPPKPKDTLMISR

SEQ ID NO: 234 - protein, 40 aa, synthetic construct
EPKSCDKTHTCPPCPAPEAEGGPSVFLFPPKPKDQLMISR

SEQ ID NO: 235 - protein, 39 aa, synthetic construct
PKSCDKTHTCPPCPAPELLGGPSVFLFPPKPKDTLMISR

SEQ ID NO: 236 - protein, 35 aa, synthetic construct
DKTHTCPPCPAPEAAGGPSVFLFPPKPKDTLMISR

SEQ ID NO: 237 - protein, 37 aa, synthetic construct
ESKYGPPCPPCPAPEAAGGPSVFLFPPKPKDTLMASR

SEQ ID NO: 238 - protein, 191 aa, synthetic construct
TPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 239 - protein, 192 aa, synthetic construct
TPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 192 aa, synthetic construct
TPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 241 - protein, 192 aa, synthetic construct
TPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 191 aa, synthetic construct
TPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 243 - protein, 191 aa, synthetic construct
TPEVTCVVVDVSQEDPEVQFNWYVDGVEVHNAKTKPREEQFNSTYRVVSVLTVLKQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 244 - protein, 191 aa, synthetic construct
TPEVTCVVVDVSQEDPEVQFNWYVDGVEVHNAKTKPREEQFNSTYRVVSVLTVLMQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 245 - protein, 191 aa, synthetic construct
TPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLG

SEQ ID NO: 246 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 247 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 248 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNF

SEQ ID NO: 249 - protein, 223 aa, unidentified
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 250 - protein, 222 aa, synthetic construct
GIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTN

SEQ ID NO: 251 - protein, 375 aa, synthetic construct
TFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 252 - protein, 219 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNK

SEQ ID NO: 253 - protein, 197 aa, synthetic construct
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKK

SEQ ID NO: 254 - protein, 223 aa, synthetic construct
RVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 255 - protein, 219 aa, synthetic construct
RVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 256 - protein, 197 aa, synthetic construct
TNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKK

SEQ ID NO: 257 - protein, 223 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 258 - protein, 219 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 259 - protein, 240 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKK

SEQ ID NO: 260 - protein, 211 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKK

SEQ ID NO: 261 - protein, 200 aa, synthetic construct
PNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKK

SEQ ID NO: 262 - protein, 192 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRV

SEQ ID NO: 263 - protein, 200 aa, synthetic construct
PNITNLCPFDEVFNATKFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKK

SEQ ID NO: 264 - protein, 200 aa, synthetic construct
PNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKK

SEQ ID NO: 265 - protein, 219 aa, synthetic construct
RVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNK

SEQ ID NO: 266 - protein, 223 aa, synthetic construct
RVQPTESIVRFPNITNLCPFDEVFNATKFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 267 - protein, 706 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 268 - protein, 685 aa, synthetic construct
MFVFLVLLPLVSTNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 269 - protein, 672 aa, synthetic construct
MFVFLVLLPLVSTNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 270 - protein, 711 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 271 - protein, 698 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 272 - protein, 706 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 273 - protein, 706 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLMQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 274 - protein, 706 aa, synthetic construct; identical to SEQ ID NO: 272

SEQ ID NO: 275 - protein, 706 aa, synthetic construct; identical to SEQ ID NO: 273

SEQ ID NO: 276 - protein, 711 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 277 - protein, 711 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLMQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 278 - protein, 721 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 279 - protein, 716 aa, synthetic construct
MFVFLVLLPLVSRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 280 - protein, 716 aa, synthetic construct
MFVFLVLLPLVSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 281 - protein, 711 aa, synthetic construct
MFVFLVLLPLVSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 282 - protein, 716 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 283 - protein, 717 aa, synthetic construct
MHSSALLCCLVLLTGVRARVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 284 - protein, 716 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 285 - protein, 722 aa, synthetic construct
MHSSALLCCLVLLTGVRARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 286 - protein, 712 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 287 - protein, 733 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKK

SEQ ID NO: 288 - protein, 704 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKK

SEQ ID NO: 289 - protein, 693 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKK

SEQ ID NO: 290 - protein, 685 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRV

SEQ ID NO: 291 - protein, 693 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSPNITNLCPFDEVFNATKFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKK

SEQ ID NO: 292 - protein, 693 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKK